(19) United States
(12) Patent Application Publication
(10) Pub. No.: US 2018/0125993 A1
(43) Pub. Date: May 10, 2018

(54) TRAIL RECEPTOR-BINDING AGENTS AND USES OF SAME

(71) Applicant: BEIJING COTIMES BIOTECH COMPANY LIMITED, Beijing (CN)

(72) Inventors: Enyun SHENG, Beijing (CN); Zhe LI, Beijing (CN); Jiagen ZHAO, Beijing (CN)

(73) Assignee: BEIJING COTIMES BIOTECH COMPANY LIMITED, Beijing (CN)

(21) Appl. No.: 15/526,759

(22) PCT Filed: Nov. 14, 2014
(86) PCT No.: PCT/CN2014/091203

§ 371 Date: May 14, 2017

(51) Int. Cl. A61K 47/68; A61K 39/395; C07K 16/28; A61P 35/00; G01N 33/574

(52) U.S. Cl. CPC A61K 47/6803; A61K 39/39558; A61K 2039/505; A61P 35/00; G01N 33/574; C07K 16/2878

(57) ABSTRACT

The present invention discloses TRAIL receptor-binding agents, their therapeutic effects on tumor inhibition in vitro and in vivo, alone or in combination with various chemotherapeutic agents. In particular, the present invention discloses methods for the treatment of cancer, comprising administrating a TRAIL receptor binding agent of the present technology, alone or in combination with a chemotherapeutic agent.

### CROSS-REFERENCE TO RELATED APPLICATIONS

This application is a U.S. National Phase of International Patent Application No. PCT/CN2014/091203, filed Nov. 14, 2014, the content of which is incorporated by reference herein in its entirety for all purposes.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 783252000200SeqList.txt, date recorded: Oct. 16, 2017, size: 17,719 bytes).

### FIELD OF THE INVENTION

This disclosure relates generally to the therapeutic uses of TRAIL receptor-binding agents. In particular, the present disclosure relates to methods and compositions including CTB006, an anti-TRAIL-R2 (DR5) antibody, and its therapeutic effects on tumor inhibition in vito and in vivo, alone or in combination with various chemotherapic agents.

### BACKGROUND

TRAIL was identified in the 90s of the last century and is known to play an important role in immune surveillance of tumor cells. Activated T lymphocytes and NK cells express high levels of TRAIL, which arms these immune competent cells with the ability to kill tumor cells. Animal studies indicate that knockout of TRAIL leads to increased incidence of tumors with age. Therefore, defective or insufficient expression of TRAIL might be a factor for tumorigenesis.

Soon after TRAIL was discovered, attention was directed to its potential as an anti-cancer agent. This was based on the ability of TRAIL to selectively kill tumor cells but not normal cells. The anti-tumor efficacy of TRAIL can be significantly enhanced by many current cancer therapies (for example, chemotherapy and radiation therapy), and in addition, TRAIL can also sensitize tumor cells and increase the susceptibility of tumor cells to chemotherapy and radiation therapy. Therefore, the combination of TRAIL with chemotherapy and/or radiation therapy holds promise as an effective anti-tumor therapy.

TRAIL is a member of the TNF family of proteins. A feature of some proteins of this family is their ability to induce apoptosis like TNF-α and Fas ligand. However, due to their toxic side effect, TNF-α and Fas ligand have limited value for clinical application. In contrast, because TRAIL exhibits selective killing of tumor cells, its clinical value is obvious. To date, five receptors for TRAIL have been identified, two of which, DR4 (TRAIL-R1) and DR5 (TRAIL-R2), are capable of transducing the apoptosis signal while the other three DcR1 (TRAIL-R3), DcR2 (TRAIL-R4), and osteoprotegerin (OPG) do not transduce the apoptosis signal. All five receptors for TRAIL share significant homology in their extracellular ligand binding domains. The intracellular segments of both DR4 and DR5 contain a conserved functional domain, the so called “death domain”, which is responsible for transducing apoptosis signals.

### SUMMARY

The present technology relates to methods for treating cancer in a subject in need thereof. In some embodiments, the method includes (a) administering to the subject a composition comprising a monoclonal antibody having the same epitope specificity as the mouse-mouse hybridoma CTB006 having CGMCC Accession No. 1691; and (b) simultaneously, sequentially or separately administering to the subject a chemotherapeutic agent.

In some embodiments, the antibody comprises heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5. In some embodiments, the antibody is a human chimeric antibody. In some embodiments, the human chimeric antibody comprises the heavy chain amino acid sequence set forth in SEQ ID NO: 14 and the light chain amino acid sequence set forth in SEQ ID NO: 12. In some embodiments, the cancer includes one or more of liver cancer, colon cancer, breast cancer, ovarian cancer, and leukemia.

In some embodiments, the chemotherapeutic agent includes one or more of 5-fluorouracil and taxol. In some embodiments, the chemotherapeutic agent is 5-fluorouracil. In some embodiments, the chemotherapeutic agent is taxol.

In another aspect, disclosed herein are methods for selectively inducing apoptosis in cells expressing a TRAIL-R2 polypeptide. In some embodiments, the methods include (a) identifying cells expressing the TRAIL-R2 polypeptide; and (b) contacting the cells with a monoclonal antibody having the same epitope specificity as the mouse-mouse hybridoma CTB006 having CGMCC Accession No. 1691.

In some embodiments, the antibody comprises heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5. In some embodiments, the antibody is a human chimeric antibody. In some embodiments, the human chimeric antibody includes the heavy chain amino acid sequence set forth in SEQ ID NO: 14 and the light chain amino acid sequence set forth in SEQ ID NO: 12. In some embodiments, the TRAIL-R2-expressing cells are cancer cells. In some embodiments, the cancer cells include one or more of liver cancer cells, colon cancer cells, breast cancer cells, ovarian cancer cells, and leukemia cells.

In some embodiments, the methods includes contacting the cells with a chemotherapeutic agent. In some embodiments, the chemotherapeutic agent includes one or more of 5-fluorouracil and taxol. In some embodiments, the chemotherapeutic agent is 5-fluorouracil. In some embodiments, the chemotherapeutic agent is taxol.

In another aspect, the provided herein are methods for treating cancer in a subject in need thereof. In some embodiments, the methods include administering to the subject a composition comprising a monoclonal antibody having the same epitope specificity as the mouse-mouse hybridoma CTB006 having CGMCC Accession No. 1691, wherein the subject is identified as having a tumor expressing TRAIL-R2.

In some embodiments, the antibody comprises heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5. In some embodiments, the antibody is a human chimeric antibody. In some embodiments, the human chimeric antibody comprises the heavy chain amino acid sequence set forth in SEQ ID NO: 14 and the light chain amino acid sequence set forth in SEQ ID NO: 12. In some embodiments, the cancer includes one or more of liver cancer, colon cancer, breast cancer, ovarian cancer, and leukemia.

In some embodiments, the methods further comprising simultaneously, sequentially or separately administering to the subject a chemotherapeutic agent. In some embodiments, the chemotherapeutic agent is 5-fluorouracil. In some embodiments, the chemotherapeutic agent is taxol.

In another aspect, the disclosure provides an in vitro method of identifying a subject amenable to cancer treatment, wherein the cancer treatment includes (a) administering to the subject a composition comprising a monoclonal antibody having the same epitope specificity as the mouse-mouse hybridoma CTB006 having CGMCC Accession No. 1691; and (b) simultaneously, sequentially or separately administering to the subject a chemotherapeutic agent. In some embodiments, the method of identifying the subject includes contacting a tumor sample from the patient with a monoclonal antibody having the same epitope specificity as the mouse-mouse hybridoma CTB006 having CGMCC Accession No. 1691. In some embodiments, the antibody comprises heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5. In some embodiments, the antibody is a human chimeric antibody. In some embodiments, the human chimeric antibody comprises the heavy chain amino acid sequence set forth in SEQ ID NO: 14 and the light chain amino acid sequence set forth in SEQ ID NO: 12. In some embodiments, the cancer includes one or more of liver cancer, colon cancer, breast cancer, ovarian cancer, and leukemia.

In another aspect, the disclosure provides a diagnostic kit for determining whether a subject is amendable to cancer treatment, wherein the cancer treatment includes (a) administering to the subject a composition comprising a monoclonal antibody having the same epitope specificity as the mouse-mouse hybridoma CTB006 having CGMCC Accession No. 1691; and (b) simultaneously, sequentially or separately administering to the subject a chemotherapeutic agent. In some embodiments, the kit includes a monoclonal antibody having the same epitope specificity as the mouse-mouse hybridoma CTB006 having CGMCC Accession No. 1691.

In some embodiments, the antibody comprises heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5. In some embodiments, the antibody is a human chimeric antibody. In some embodiments, the human chimeric antibody comprises the heavy chain amino acid sequence set forth in SEQ ID NO: 14 and the light chain amino acid sequence set forth in SEQ ID NO: 12. In some embodiments, the cancer includes one or more of liver cancer, colon cancer, breast cancer, ovarian cancer, and leukemia.

In another aspect, the disclosure provides for the use of an antibody in the preparation of a medicament for treating cancer in a subject identified as having a tumor expressing TRAIL-R2. In some embodiments, the antibody comprises a monoclonal antibody having the same epitope specificity as the mouse-mouse hybridoma CTB006 having CGMCC Accession No. 1691.

In some embodiments, the antibody comprises heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5. In some embodiments, the antibody is a human chimeric antibody. In some embodiments, the human chimeric antibody comprises the heavy chain amino acid sequence set forth in SEQ ID NO: 14 and the light chain amino acid sequence set forth in SEQ ID NO: 12. In some embodiments, the cancer includes one or more of liver cancer, colon cancer, breast cancer, ovarian cancer, and leukemia. In some embodiments, the medicament further comprises one or more chemotherapeutic agent. In some embodiments, the chemotherapeutic agent is 5-fluorouracil. In some embodiments, the chemotherapeutic agent is taxol.

In another aspect, the disclosure provides for the use of an antibody in the preparation of a medicament for selectively inducing apoptosis in cells expressing a TRAIL-R2 polypeptide. In some embodiments, the antibody includes a monoclonal antibody having the same epitope specificity as the mouse-mouse hybridoma CTB006 having CGMCC Accession No. 1691.

In some embodiments, the antibody comprises heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5. In some embodiments, the antibody is a human chimeric antibody. In some embodiments, the human chimeric antibody comprises the heavy chain amino acid sequence set forth in SEQ ID NO: 14 and the light chain amino acid sequence set forth in SEQ ID NO: 12. In some embodiments, the TRIAL-R2-expressing cells are cancer cells. In some embodiments, the cancer cells include one or more of liver cancer cells, colon cancer cells, breast cancer cells, ovarian cancer cells, and leukemia cells. In some embodiments, the medicament further comprises one or more chemotherapeutic agent. In some embodiments, the chemotherapeutic agent is 5-fluorouracil. In some embodiments, the chemotherapeutic agent is taxol.

### BRIEF DESCRIPTION OF THE DRAWINGS

The figures depict non-limiting exemplary embodiments of the technology disclosed herein and are provided to aid the reader in understanding the disclosure. In the figures and figure descriptions, HuCTB006, the human chimeric antibody, is referred to as “CTB006.”

FIGS. 1A and 1B are graphs showing binding specificity of murine CTB006, mCTB006 (FIG. 1A) and human CTB006 (FIG. 1B) determined by chemiluminescent enzyme immunoassay (CLEIA). Binding affinity was tested with each of the five TRAIL receptors, DR5, DR4, DcR1, DcR2 and OPG.

FIG. 2 is a graph showing affinity of CTB006 determined by surface plasmon resonance (SPR).

FIGS. 3A and 3B are graphs showing cytotoxicity of CTB006 or oxaliplatin on human normal tissue cell lines, WI-38 (FIG. 3A) and HFL-1 (FIG. 3B).

FIGS. 4A and B are graphs showing cytotoxicity of CTB006 or oxaliplatin on human normal tissue cell lines, HUV-EC-C (FIG. 4A) and liver differentiated cell (FIG. 4B).

FIGS. 5A and 5B are graphs showing cytotoxicity of CTB006 or oxaliplatin on human liver cancer cell lines, SK-Hep-1 (FIG. 5A) and HepG2 (FIG. 5B).

FIGS. 6A and 6B are graphs showing cytotoxicity of CTB006 or irinotecan on human colorectal cancer cell lines, Colo205 (FIG. 6A) and HCT116 (FIG. 6B).

FIGS. 7A and 7B are graphs showing cytotoxicity of CTB006 or irinotecan on human colorectal cancer cell lines, SW480 (FIG. 7A) and WiDr (FIG. 7B).

FIGS. 8A, 8B and 8C are graphs showing cytotoxicity of CTB006 or oxaliplatin on human pancreatic cancer cell lines, BXPC3 (FIG. 8A), MIA-PaCa-2 (FIG. 8B), and Panc2.03 (FIG. 8C).

FIGS. 9A and 9B are graphs showing cytotoxicity of CTB006 or paclitaxel on human lung cancer cell lines, H2122 (FIG. 9A) and SK-MES-1 (FIG. 9B).

FIGS. 10A and 10B are graphs showing cytotoxicity of CTB006 or irinotecan on human breast cancer cell lines, MDA-MB-231 (FIG. 10A) and DY36T2 (FIG. 10B).

FIGS. 11A and 11B are graphs showing cytotoxicity of CTB006 or irinotecan on human breast cancer cell lines, 2-LMP (FIG. 11A) and SUM102 (FIG. 11B).

FIG. 12 is a graph showing cytotoxicity of CTB006 or cisplatin on human ovarian cancer cell line, OVCAR3.

FIGS. 13A and 13B are graphs showing cytotoxicity of CTB006 or methotrexate on human acute T-lymphoblastic leukemia cell lines, Molt-4 (FIG. 13A) and Jurkat (FIG. 13B).

FIG. 14 is a graph showing the cytotoxic effect of CTB006 or 5-FU, and the synertistic cytotoxic effect of the combination of CTB006 and 5-FU on BGC823 stomach cancer cells.

FIG. 15 is a graph showing the cytotoxic effect of CTB006 or 5-FU, and the synertistic cytotoxic effect of the combination of CTB006 and 5-FU on Panc2.03 pancreatic cancer cells.

FIG. 16 is a graph showing the cytotoxic effect of CTB006 or paclitaxel, and the synertistic cytotoxic effect of the combination of CTB006 and paclitaxel on A549 lung cancer cells.

FIG. 17 is a graph showing the cytotoxic effect of CTB006 or paclitaxel, and the synertistic cytotoxic effect of the combination of CTB006 and paclitaxel on H460 lung cancer cells.

FIG. 18 is a graph showing the cytotoxic effect of CTB006 or 5-FU, and the synertistic cytotoxic effect of the combination of CTB006 and 5-FU on Huh-7 liver cancer cells.

FIG. 19 is a graph showing the cytotoxic effect of CTB006 or gemcitabine, and the synertistic cytotoxic effect of the combination of CTB006 and gemcitabine on 7402 liver cancer cells.

FIGS. 20A and 20B are graphs showing the cytotoxic effect of each of CTB006, sorafenib, lapatinib, or erlotinib alone, and the synergistic cytotoxic effect of CTB006 combined with sorafenib, lapatinib, or erlotinib, on SW480 colorectal cancer cells. The cells were treated with CTB006 at 62.5, 125, 250, 500, or 1000 ng/ml. Sorafenib, lapatinib and erlotinib were each used at 10 μM (FIG. 20A) or 5 μM (FIG. 20B).

FIG. 21A-21D. FIGS. 21A and 21B are graphs showing the cytotoxic effect of each of CTB006, sorafenib, lapatinib, or erlotinib alone, and and the synergistic cytotoxic effect of CTB006 combined with sorafenib, lapatinib, or erlotinib, on Widr colorectal cancer cells. FIGS. 21C and 21D are graphs showing the cytotoxic effect of each of CTB006, erbitux or herceptin alone, and the synergistic cytotoxic effect of CTB006 combined with erbitux or herceptin, on Widr colorectal cancer cells. The cells were treated with CTB006 at 62.5, 125, 250, 500, or 1000 ng/ml, and sorafenib, lapatinib, and erlotinib at 10 μM (21A) or 5 μM (21B), erbitus or herceptin at 1 μg/ml (FIG. 21C) or 0.1 μg/ml (FIG. 21D).

FIG. 22A-22C. FIGS. 22A and 22B are graphs showing of each of CTB006, sorafenib, lapatinib, or erlotinib alone, and and the synergistic cytotoxic effect of CTB006 combined with sorafenib, lapatinib, or erlotinib, on BGC823 stomach cancer cells. FIG. 22C is a graph showing the cytotoxic effect of each of CTB006, erbitux or herceptin alone, and the synergistic cytotoxic effect of CTB006 combined with erbitux or herceptin, on BGC823 stomach cancer cells. The cells were treated with CTB006 at 62.5, 125, 250, 500, or 1000 ng/ml, and sorafenib, lapatinib, and erlotinib at 10 μM (FIG. 22A) or 5 μM (FIG. 22B), erbitus or herceptin at 10 μg/ml or 1 μg/ml (FIG. 22C).

FIG. 23A-23C. FIGS. 23A and 23B are graphs showing of each of CTB006, sorafenib, lapatinib, or erlotinib alone, and and the synergistic cytotoxic effect of CTB006 combined with sorafenib, lapatinib, or erlotinib, on NUGC3 stomach cancer cells. FIG. 23C is a graph showing the cytotoxic effect of each of CTB006, erbitux or herceptin alone, and the cytotoxic effect of CTB006 combined with erbitux or herceptin, on NUGC3 stomach cancer cells. The cells were treated with CTB006 at 62.5, 125, 250, 500, or 1000 ng/ml, and sorafenib, lapatinib, and erlotinib at 10 μM (FIG. 23A) or 5 μM (FIG. 23B), erbitus or herceptin at 10 μg/ml or 1 μg/ml (FIG. 23C).

FIGS. 24A and 24B are graphs showing of each of CTB006, sorafenib, lapatinib, or erlotinib alone, and and the synergistic cytotoxic effect of CTB006 combined with sorafenib, lapatinib, or erlotinib, on H460 lung cancer cells. The cells were treated with CTB006 at 62.5, 125, 250, 500, or 1000 ng/ml, and sorafenib, lapatinib, and erlotinib at 10 μM (FIG. 24A) or 5 μM (FIG. 24B).

FIGS. 25A and 25B are graphs showing of each of CTB006, sorafenib, lapatinib, or erlotinib alone, and and the synergistic cytotoxic effect of CTB006 combined with sorafenib, lapatinib, or erlotinib, on SK-BR3 breast cancer cells. The cells were treated with CTB006 at 62.5, 125, 250, 500, or 1000 ng/ml, and sorafenib, lapatinib, and erlotinib at 7 μM (FIG. 25A) or 3.5 μM (FIG. 25B).

FIGS. 26A and 26B are graphs showing of each of CTB006, sorafenib, lapatinib, or erlotinib alone, and and the synergistic cytotoxic effect of CTB006 combined with sorafenib, lapatinib, or erlotinib, on SUM 102 breast cancer cells. The cells were treated with CTB006 at 62.5, 125, 250, 500, or 1000 ng/ml, and sorafenib, lapatinib, and erlotinib at 7 μM (FIG. 26A) or 3.5 μM (FIG. 26B).

FIGS. 27A and 27B are graphs showing of each of CTB006, sorafenib, lapatinib, or erlotinib alone, and and the synergistic cytotoxic effect of CTB006 combined with sorafenib, lapatinib, or erlotinib, on DY36T2 breast cancer cells. The cells were treated with CTB006 at 62.5, 125, 250, 500, or 1000 ng/ml, and sorafenib, lapatinib, and erlotinib at 7 μM (FIG. 27A) or 3.5 μM (FIG. 27B).

FIGS. 28A and 28B are graphs showing the effect of CTB006 or gemicitabone treatment on tumor progression in mice using an MIA-PaCa-2 (pancreatic cancer cells) subcutaneous model. 28A shows the effect of CTB006 or gemicitabone treatment on tumor volume, and FIG. 28B shows the effect of CTB006 or gemicitabone treatment on tumor weight. The “+” signs at the bottom of FIG. 28A indicates that the mice were treated that day.

FIG. 29 is a graph showing the effect of CTB006 or gemicitabone treatment on the body weight in mice in an MIA-PaCa-2 (pancreatic cancer cells) subcutaneous model.

FIG. 30A-30C are graphs showing the effect of CTB006 or irinotecan treatment on the tumor progression in mice using a colo205 subcutaneous model. FIG. 30A shows the effect of CTB006 or irinotecan treatment on tumor volume, FIG. 30B shows the effect of CTB006 or irinotecan treatment on tumor weight, and FIG. 30C shows the effect of CTB006 or irinotecan treatment on the tumor bearing rate.

FIG. 31 is a graph showing the effect of CTB006 or irinotecan treatment on the body weight of in mice using a colo205 subcutaneous model.

FIGS. 32A and 32B are graphs showing the effect of CTB006 or irinotecan treatment on the tumor progression in mice using a WiDr subcutaneous model. FIG. 32A shows the effect of CTB006 or irinotecan treatment on tumor volume, and FIG. 32B shows the effect of CTB006 or irinotecan treatment on the tumor weights in WiDr subcutaneous model.

FIG. 33 is a graph showing the effect of CTB006 or irinotecan treatment on the body weight in mice using a WiDr subcutaneous model.

FIG. 34A-34C are graphs showing the effect of CTB006 or taxon treatment on the tumor progression in mice using an H2122 subcutaneous model. FIG. 34A shows the effect of CTB006 or taxon treatment on tumor volume, FIG. 34B shows the effect of CTB006 or taxon treatment on tumor weight, and, FIG. 34C shows the effect of CTB006 or taxon treatment on the tumor bearing rate.

FIG. 35 is a graph showing the effect of CTB006 or taxol treatment on the body weight of mice using an H2122 subcutaneous model.

FIG. 36A-36C. FIG. 36A is a graph showing the effect of the CTB006 alone, 5-FU alone, and the synergistic effect of the combination of CTB006 and 5-Fu on tumor growth in a patient primary tumor tissue (colon cancer, CS146, 2#) using a subcutaneous mouse model. FIG. 36A shows the synergistic effect of the combination of CTB006 and 5-Fu on tumor volume. FIG. 36B shows a section of tumor tissue treated with CTB0006 alone, and the pathological sections of patients primary tumor tissue CS146, HE,200×. FIG. 36C shows a section of tumor tissue treated with both CTB0006 and 5-FU, and the pathological sections of CS146, 0#, HE,200×.

FIG. 37A-37C. FIGS. 37A and 37B are graphs showing the effect of CTB0006 alone, irinotecan alone, and the synergistic effect of the combination of CTB006 and irinotecan on tumor growth in patients primary tumor tissue (colon cancer, CS182, 5#) using a subcutaneous model. FIG. 37A shows the synergistic effect of the combination of CTB006 and irinotecan on tumor volume. FIG. 37B shows the synergistic effect of the combination of CTB006 and irinotecan on the tumor weight, and FIG. 37C shows the pathological sections of CS182, 0#, HE,200×.

FIG. 38A-38E. FIG. 38A and FIG. 38B are graphs showing the effect of CTB0006 alone, irinotecan alone and the synergistic combination of CTB006 and irinotecan on tumor growth in patients primary tumor tissue (colon cancer, CS263, 1#) using a subcutaneous mouse model. FIG. 38A shows the synergistic effect of the combination of CTB006 and irinotecan on the tumor volume and FIG. 38B shows the effect of the synertistic effect of the combination of CTB006 and irinotecan on tumor weight. FIG. 38C shows the pathological sections of CS263, 0#, HE,200×; FIG. 38D shows the pathological sections of CS263, 1#, HE, 200×; and FIG. 38E shows the pathological examination results of CS263. HE, 200×, in which A) Control, B) Combination group, C) the combination group which did not show any tumor cells.

FIG. 39A-D. FIGS. 39A and 39B are graphs showing the effect of CTB0006 alone, taxol plus carboplatin, and the synergistic effect of the combination CTB006, taxol and carboplatin on tumor growth in patients primary tumor tissue (lung cancer, CS113, 3#) in a subcutaneous mouse model. FIG. 39A shows the synergtistic effect of the combination of CTB006, taxol and carboplatin on tumor volume and FIG. 39B shows the synergistic effect of the combination of CTB006, taxol and carboplatin on the tumor bearing rate. FIG. 39C shows the pathological sections of CS113, 0#, HE, 200×; and FIG. 39D shows the pathological sections of CS113, 1#, HE,200×.

FIG. 40A-D. FIG. 40A and FIG. 40B are graphs showing the effect of CTB006 alone, cisplatin alone and the synergistic effect of the combination of CTB006 and cisplatin on tumor growth in patients primary tumor tissue (lung cancer, CS225, 1#) using a subcutaneous mouse model. FIG. 40A shows the synergistic effect of the combination of CTB006 and cisplatin on tumor volume, and FIG. 40B shows the synergistic effect of the combination of CTB006 and cisplatin on tumor weight. FIG. 40C shows the pathological sections of CS225, 0#, HE,200×; and FIG. 40D shows the pathological sections of CS1225, 1#, HE,200×.

FIG. 41 is a graph showing the relationship between the concentration of DR5 in human tumor cell lysis solution and the in vitro cytotoxic sensitivity of CTB006, showing the relativity between concentration of DR5 in human tumor cell lysis solution and in vitro cytotoxic sensitivity of CTB006. The cells with higher DR5 concertration show more effective killing effects in vitro. The threshold of the ‘responder’ was 0.208 ng/ml, and for the ‘partialresponder’ was 0.109 ng/ml. The detection limit was 0.0256 ng/ml.

FIG. 42A-I. FIGS. 42A, B, D, E, G and H are tissue sections. This figure shows the compatibility of the DR5 expression detected by CLEIA Kit and IHC (some colon cancer tissues and the relative adjacent tissues were used as example). In this figure, the left pathological figures (i.e., FIGS. 42A, 42D, and 42G) are the pathological sections of tumor, and the right pathological figures (i.e., FIGS. 42B, 42E, and 42H) are the relative adjacent tissues. The results of CLEIA Kit indicated DR5 expression. From all of the figures, the higher DR5 expression of the tumor detected by CLELA Kit corresponds to the pathological sections of tumor, which is shown as ++˜+++.

### DETAILED DESCRIPTION OF THE INVENTION

### I. General

It is to be appreciated that certain aspects, modes, embodiments, variations and features of the technology disclosed herein are described below in various levels of detail in order to provide a substantial understanding technology.

The present technology provides TRAIL receptor-binding agents (e.g., antibodies) useful for inducing apoptosis in cells expressing the TRAIL-R2 polypeptide and for treating cancer.

Because the apoptosis-inducing function of TRAIL is mediated by its receptors, research on TRAIL receptor system has been extensive. Early studies suggest that many normal cells may express the death receptors (TRAIL-R1 and TRAIL-R2) for TRAIL at the transcriptional level. With the availability of anti-death receptor antibodies, it has been believed that normal cells and tissues express very low levels of cell surface TRAIL-R1 and TRAIL-R2. In contrast, normal cells and tissues may express high levels of TRAIL-R3 and TRAIL-R4. This differential expression of different TRAIL receptors in normal cell may be a protective mechanism for normal cells to escape from TRAIL killing Different from normal cells, most transformed tumor cells express high levels of TRAIL-R1 and TRAIL-R2 whereas the expression levels of TRAIL-R3 and TRAIL-R4 are very low. Thus, most tumor cells are susceptible to TRAIL-mediated killing The differentially expressed TRAIL receptors between normal and tumor cells explain the selectivity of TRAIL.

Many pre-clinical studies have confirmed that TRAIL is a safe and effective therapeutic agent for treatment of cancer. It has been shown that the systemic administration of the trimerized soluble TRAIL did not cause toxicity in experimental animals yet was able to induce regression of implanted tumors. It is even more encouraging that when TRAIL is combined with chemotherapy or radiation therapy, its anti-tumor efficacy is significantly enhanced. This synergistic effect has been demonstrated by many in vitro and in vivo experiments. In addition, TRAIL can increase the sensitivity of tumor cells to chemotherapy and radiation therapy. Because tumor cell resistance to chemotherapy and radiation therapy has been a major obstacle in treatment of cancer, the ability of TRAIL to prevent or reverse chemo or radiation resistance might be a significant advance in future cancer therapy.

However, as a therapeutic agent, TRAIL has several disadvantages. First, TRAIL has at least five receptors including both death receptors and decoy receptors, therefore lacking the selectivity to the receptors. Particularly, it is hard to predict the apoptosis-inducing capability of TRAIL when cancer cells express differentiated death receptors and decoy receptors. Second, the recombinant TRAIL has very short in vivo half-life, which limits the effective dose and anti-cancer efficacy of TRAIL in vivo. It is not convenient that patients usually receive reapeated and large doses of TRAIL. Third, it is concerned that certain forms of recombinant TRAIL have potential hepatocyte toxicity.

These limitations of TRAIL as a therapeutic agent led to development of the alternatives to TRAIL. Monoclonal antibodies may selectively target the death receptors of TRAIL, which might be a more effective and safe strategy to cancer treatment.

In the 25 years since the first monoclonal antibody was generated, monoclonal antibodies have demonstrated a great impact in cancer treatment. Most of those clinically effective monoclonal antibodies target antigens or receptors that are highly expressed on cancer cell surface, and block the growth signals required for tumor growth. These antibodies kill tumor cells through activation of compliments and antibody-dependent cytotoxicity (ADCC). In addition, monoclonal antibodies may be used as a tracing molecule, when conjugated with radioisotopes, toxins and drugs, to bring these therapeutic agents to cancer tissues and enhance anti-cancer efficacy.

The generation of TRAIL-R1 or TRAIL-R2 specific monoclonal antibody to replace TRAIL for cancer therapy has been successful. Several such antibodies have been in clinical trials. Preliminary results demonstrate that these antibodies not only have strong anticancer efficacy but also are safe compared to TRAIL.

Japanese pharmaceutical company, Sankyo, first developed an anti-TRAIL-R2 antibody, TRA-8. Ichikawa et al. used TRAIL-R2-Fc fusion protein as immunogen to immunize Balb/c mice. While TRA-8 did not induce apoptosis of normal cells, many tumor cells were highly susceptible to TRA-8-induced apoptosis. Although mRNA of TRAIL-R2 is widely distributed in normal tissues, the TRAIL-R2 protein was not detectable in normal tissues including live, lung, breast, kidney, spleen, ovary, hear and pancreas. However, cancer cells in these tissues expressed high levels of TRAIL-R2 protein. In addition, normal glial cells and peripheral blood cells expressed very low levels of TRAIL-R2, and are not susceptible to TRA-8-induced apoptosis, whereas gliloma cells and leukemia cells expressed high levels and are very susceptible to TRA-8-induced apoptosis. TRA-8 also exhibited several folds higher apoptosis-inducing capability than TRAIL in induction of apoptosis of tumor cells. Importantly, TRA-8 did not induce apoptosis of normal hepatocytes. When combined with chemotherapy or radiation therapy, the anti-cancer efficacy of TRA-8 is significantly enhanced. TRA-8 is currently in phase I clinical trial.

Human Genome Sciences carried out phase I trial of an anti-TRAIL-R1 antibody. Preliminary data indicate that patients tolerated the compound well, and a positive response was observed in several patients, suggesting that anti-TRAIL-R1 is a safe and effective therapeutic agent.

The TRAIL receptor-binding agents of the present technology, (e.g., monoclonal antibodies having the same epitope specificity as the mouse-mouse hybridoma CTB006 having CGMCC Accession No. 1691; monoclonal antibodies having a heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; a monoclonal antibody having the heavy chain amino acid sequence set forth in SEQ ID NO: 14 and the light chain amino acid sequence set forth in SEQ ID NO: 12; the HuCTB006 antibody), are useful, alone or in combination with other active agents, e.g., chemotherapeutic drugs, to modulate TRAIL receptor-mediated function. In particular, the TRAIL receptor binding agents of the present technology show significant and unexpected levels of synergy when combined with chemotherapeutic agents, e.g., 5-fluorouracil and/or taxol, in killing cancer cells. In addition, the TRAIL receptor-binding agents of the present technology are useful to detect a TRAIL receptor polypeptide (a.k.a., the target polypeptide) in a test sample (e.g., a patient sample). TRAIL receptor-binding agents are useful to diagnose, prevent and/or treat a TRAIL receptor-related medical condition in subjects in need thereof. The TRAIL receptor-binding agents (e.g., antibodies) of the present technology provide a unique biological function and anti-cancer activity. Although soluble TRAIL has been shown to be effective in induction of apoptosis of tumor cells in vivo, the killing activity appeared to be very low due to its hort half-life, and large (and repeated) doses are often required. The binding agents according to the present technology are pharmaceutically more effective compared to TRAIL and other monospecific anti-TRAIL-R2 antibodies.

The various aspects of the present technology additionally relate to diagnostic methods and kits that use the TRAIL receptor-binding agents of the present technology to identify individuals predisposed to a medical condition or to classify individuals with regard to drug responsiveness, side effects, or optimal drug dose. In other aspects, the technology provides methods for the use of TRAIL receptor-binding agents to prevent or treat TRAIL receptor-mediated disorders, various cancers, as well as to screen and/or validate ligands, e.g., small molecules that bind a TRAIL receptor polypeptide. Accordingly, various particular embodiments that illustrate these aspects follow.

The details of one or more embodiments of the technology are set forth in the accompanying description below. The skilled artisan will recognize that methods and materials similar or equivalent to those described herein can be used in the practice or testing of the present technology. Other features, objects, and advantages of the technology will be apparent from the description and the claims. Generally, enzymatic reactions and purification steps are performed according to the manufacturer's specifications. The techniques and procedures are generally performed according to conventional methods in the art and various general references (see generally, Sambrook et al., Molecular Cloning: A Laboratory Manual, 2d Ed. (Cold Spring Harbor Laboratory Press, Cold Spring Harbor, N.Y., 1989), which are provided throughout this document.

### II. Definitions and Abbreviations

Unless defined otherwise, all technical and scientific terms used herein generally have the same meaning as commonly understood by one of ordinary skill in the art to which this technology belongs. As used in this specification and the appended claims, the singular forms “a,” “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well known and commonly employed in the art. Standard techniques are used for nucleic acid and peptide synthesis. Standard techniques, or modifications thereof, are used for chemical syntheses and chemical analyses. All references cited herein are incorporated herein by reference in their entireties and for all purposes to the same extent as if each individual publication, patent, or patent application was specifically and individually incorporated by reference in its entirety for all purposes.

Abbreviations of select biochemistry and haematology terms are summarized below in Table A and Table B, respectively.

TABLE A
Select Biochemistry Terms

| | |
|---|---|
| Alb | albumin |
| TP | total protein |
| Tchol | total cholesterol |
| TG | triglyceride |
| Tbil | total bilirubin |
| Glu | glucose |
| Na | sodium |
| Ca | calcium |
| K | potassium |
| Cl | chlorine |

TABLE B
Select Hematology Terms

| | |
|---|---|
| RBC | red blood cell count |
| Hb | hemoglobin |
| Hct | hematocrit |
| MCV | mean corpuscular volume |
| MCH | mean corpuscular hemoglobin |
| MCHC | mean corpuscular hemoglobin concentration |
| RDW | red(cell) distribution width |
| Plat | platelet |
| MPV | mean platelet volume |
| PDW | platelet distribution width |
| WBC | white blood cell count |
| WBC-D.C | white blood cell differential count |
| Ret | reticulocytes |

The definitions of certain terms as used in this specification are provided below. Definitions of other terms may be found in the Illustrated Dictionary of Immunology, 2nd Edition (Cruse, J. M. and Lewis, R. E., Eds., Boca Raton, Fla.: CRC Press, 1995).

The paired terms “DR4” and “TRAIL-R1,” “DR5” and “TRAIL-R2,” “DcR1” and “TRAIL-R3” and “DcR2” and “TRAIL-R4” can be used interchangeably. Unless indicated otherwise, the terms when used herein refer to human protein and gene. Murine versions of molecules may be preceeded by m.

As used herein, the term “biological activity” of the TRAIL receptor-binding agents (e.g., antibodies) of the present technology, or variants or fragments thereof, refers to the ability of the agent to (1) specifically bind TRAIL-R2 (DR5); (2) induce cancer cell death, in vito and in vivo; and (3) demonstrate a synergistic effect with chemotherapeutic agents such as taxol and 5-fluorouracil, in inducing cancer cell death in vitro and in vivo.

As used herein, the term “synergy” or “synergist” refers to an effect arising between two or more agents, entities, factors, or substances that produces an effect greater than the sum of their individual effects. In some embodiment, syngegy between biologically active agents, such as TRAIL receptor binding agents of the present technology (e.g., monoclonal antibodies having the same epitope specificity as the mouse-mouse hybridoma CTB006 having CGMCC Accession No. 1691; monoclonal antibodies having a heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; a monoclonal antibody having the heavy chain amino acid sequence set forth in SEQ ID NO: 14 and the light chain amino acid sequence set forth in SEQ ID NO: 12; the HuCTB006 antibody) and a chemotherapeutic agent is determined via the coefficient of drug interaction (i.e., CDI) (see e.g., Cao S S, et al., Potentiation of antimetabolite antitumor activity in vivo by dipyridamole and amphotericin B. Cancer Chemother Pharmacol 1989; 24: 181-186). In some embodiments, CDI is calculated as follows: CDI=AB/A×B. According to the chemiluminescence of each group, AB is the ratio of the combination groups to the control group; A or B is the ratio of the single agent group to the control group. The drug combination is synergistic when the CDI value is less than 1. In determining a synergistic interaction between two or more components, in some embodiments, the optimum range for the effect, and the absolute dose range of each component for the effect, may be measured by administration of the components over different w/w ratio ranges and/or different doses to patients in need of treatment. The observation of synergy in one species can be predictive of the effect in other species, and the results of such studies can be used to predict effective dose.

As used herein, the term “significant synergy” or “significantly synergistic” refers to an effect arising between two or more agents, entities, factors, or substances that produces an effect statistically significantly greater than the sum of their individual effects. By way of example, but not by way of limitation, a drug combination is significantly synergistic when the CDI value is less than 0.7.

As used herein, the term “additive” refers to an effect arising between two or more agents, entities, factors, or substances that produces an effect equal to the sum of their individual effects. By way of example, but not by way of limitation, a drug combination is additive when the CDI value is equal to 1.

As used herein, the term “antergy” or “antagonistic” refers to an effect arising between two or more agents, entities, factors, or substances that produces an effect less than the sum of their individual effects. By way of example, but not by way of limitation, a drug combination is additive when the CDI value is greater than 1.

As used herein, the term “TRAIL receptor” refers to a member of the TNF receptor family Human TRAIL receptors are cell surface receptors for TRAIL (AP02 ligand). To date, five receptors for TRAIL have been identified, two of which, DR4 (TRAIL-R1; CD261 or Death Receptor 4) and DR5 (TRAIL-R2; CD262 or Death Receptor 5), are capable of transducing the apoptosis signal while the other three DcR1 (TRAIL-R3; CD263 or Decoy Receptor 1), DcR2 (TRAIL-R4; CD264 or Decoy Receptor 2), and osteoprotegerin (OPG) do not transduce the apoptosis signal. Binding of trimeric TRAIL to TRAIL R1 or TRAIL R2 induces apoptosis by oligomerization of these receptors. TRAIL R1 and TRAIL R2 are composed of extracellular cysteine-rich domains, a transmembrane domain and a cytoplasmic death domain TRAIL R3 and TRAIL R4 also have extracellular cysteine-rich domains but TRAIL R3 lacks cytoplasmic death domain and TRAIL R4 has a truncated one. All five receptors for TRAIL share significant homology in their extracellular ligand binding domains. The intracellular segments of both DR4 and DR5 contain a conserved functional domain, the so called “death domain,” which is responsible for transducing apoptosis signals.

As used herein, the administration of an agent or drug to a subject includes self-administration and the administration by another. It is also to be appreciated that the various modes of treatment or prevention of medical conditions as described are intended to mean “substantial,” which includes total but also less than total treatment or prevention, and wherein some biologically or medically relevant result is achieved.

As used herein, the term “amino acid” includes naturally-occurring amino acids and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine Amino acid analogs refers to compounds that have the same basic chemical structure as a naturally-occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally-occurring amino acid Amino acid mimetics refers to chemical compounds that have a structure that is different from the general chemical structure of an amino acid, but that functions in a manner similar to a naturally-occurring amino acid Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

As used herein, the term “antibody” means a polypeptide comprising a framework region from an immunoglobulin gene or fragments thereof that specifically binds and recognizes an antigen, e.g., a TRAIL receptor polypeptide. Use of the term antibody is meant to include whole antibodies, including single-chain whole antibodies, and antigen-binding fragments thereof. The term “antibody” includes bispecific antibodies and multispecific antibodies so long as they exhibit the desired biological activity or function.

As used herein, the term “antibody-related polypeptide” means antigen-binding antibody fragments, including single-chain antibodies, that can comprise the variable region(s) alone, or in combination, with all or part of the following polypeptide elements: hinge region, CH₁, CH₂, and CH₃ domains of an antibody molecule. Also included in the disclosure are any combinations of variable region(s) and hinge region, CH₁, CH₂, and CH₃ domains. Antibody-related molecules useful as binding agents of the present technology include, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR). As such “antibody fragments” can comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments. Single-chain antibody molecules may comprise a polymer with a number of individual molecules, for example, dimmer, trimer or other polymers.

As used herein, the term “biological sample” means sample material derived from or contacted by living cells. The term “biological sample” is intended to include tissues, cells and biological fluids isolated from a subject, as well as tissues, cells and fluids present within a subject. Biological samples of the present technology include, e.g., but are not limited to, whole blood, plasma, semen, saliva, tears, urine, fecal material, sweat, buccal, skin, cerebrospinal fluid, and hair. Biological samples can also be obtained from biopsies of internal organs or from cancers. Biological samples can be obtained from subjects for diagnosis or research or can be obtained from undiseased individuals, as controls or for basic research.

As used herein, the term “CDR-grafted antibody” means an antibody in which at least one CDR of an “acceptor” antibody is replaced by a CDR “graft” from a “donor” antibody possessing a desirable antigen specificity.

As used herein, the term “chimeric antibody” means an antibody in which the Fc constant region of a monoclonal antibody from one species (e.g., a mouse Fc constant region) is replaced, using recombinant DNA techniques, with an Fc constant region from an antibody of another species (e.g., a human Fc constant region). See generally, Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 125,023; Better et al., Science 240: 1041-1043, 1988; Liu et al., Proc Natl Acad Sci USA 84: 3439-3443, 1987; Liu et al., J Immunol 139: 3521-3526, 1987; Sun et al., Proc Natl Acad Sci USA 84: 214-218, 1987; Nishimura et al., Cancer Res 47: 999-1005, 1987; Wood et al., Nature 314: 446-449, 1885; and Shaw et al., J Natl Cancer Inst 80: 1553-1559, 1988.

As used herein, the term “comparison window” means a segment of any one of the number of contiguous positions selected from the group consisting of from 20 to 600 amino acids or nucleotides, usually about 50 to about 200, more usually about 100 to about 150 in which a sequence can be compared to a reference sequence of the same number of contiguous positions after the two sequences are optimally aligned.

As used herein, the term “consensus FR” means a framework (FR) antibody region in a consensus immunoglobulin sequence. The FR regions of an antibody do not contact the antigen.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). That is, in a family of proteins, each position in the consensus sequence is occupied by the amino acid occurring most frequently at that position in the family. If two amino acids occur equally frequently, either can be included in the consensus sequence.

As used herein, the term “contacted” when applied to a cell refers to the process by which a TRAIL receptor-binding agent of the present technology, antibody, antibody composition, cytotoxic agent or moiety, gene, protein and/or antisense sequence, is delivered to a target cell or is placed in direct proximity with the target cell. This delivery can be in vitro or in vivo and can involve the use of a recombinant vector system.

As used herein, the term “cytotoxic moiety” means a moiety that inhibits cell growth or promotes cell death when proximate to or absorbed by a cell. Suitable cytotoxic moieties in this regard include radioactive agents or isotopes (radionuclides), chemotoxic agents such as differentiation inducers, inhibitors and small chemotoxic drugs, toxin proteins and derivatives thereof, as well as nucleotide sequences (or their antisense sequence). Therefore, the cytotoxic moiety can be, by way of non-limiting example, a chemotherapeutic agent, a photoactivated toxin or a radioactive agent.

As used herein, the term “diabodies” refers to small antibody fragments with two antigen-binding sites, which fragments comprise a heavy-chain variable domain (V_{H}) connected to a light-chain variable domain (V_{L}) in the same polypeptide chain (V_{H} V_{L}). By using a linker that is too short to allow pairing between the two domains on the same chain, the domains are forced to pair with the complementary domains of another chain and create two antigen binding sites. Diabodies are described more fully in, e.g., EP 404,097; WO 93/11161; and 30 Hollinger et al., Proc. Natl. Acad. Sci. USA, 90: 6444-6448 (1993).

As used herein, the term “effector cell” means an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fc receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcaR are involved in specific killing of target cells and presenting antigens to other components of the immune system, or binding to cells that present antigens. An effector cell can also phagocytose a target antigen, target cell, metastatic cancer cell, or microorganism.

As used herein, the term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

To screen for TRAIL receptor-bining agents which bind to a specific epitope, a routine cross-blocking assay such as that described in Antibodies, A Laboratory Manual, Cold Spring Harbor Laboratory, Ed Harlow and David Lane (1988), can be performed. This assay can be used to determine if a test TRAIL receptor binding agent binds the same site or epitope as a TRAIL-R2 antibody of the present technology. Alternatively, or additionally, epitope mapping can be performed by methods known in the art. For example, the antibody sequence can be mutagenized such as by alanine scanning, to identify contact residues. In a different method, peptides corresponding to different regions of TRAIL-R2 can be used in competition assays with the test antibodies or with a test antibody and an antibody with a characterized or known epitope.

As used herein, the term “effective amount” of a composition, is a quantity sufficient to achieve a desired therapeutic and/or prophylactic effect, e.g., an amount which results in the prevention of, or a decrease in, the symptoms associated with a disease that is being treated, e.g., the diseases associated with target polypeptide. The amount of a composition of the present technology administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions of the present technology can also be administered in combination with each other, or with one or more additional therapeutic compounds.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, a “fusion polypeptide” comprises a TRAIL receptor polypeptide operatively-linked to a polypeptide having an amino acid sequence corresponding to a polypeptide that is not substantially homologous to the TRAIL receptor polypeptide, e.g., a polypeptide that is different from the TRAIL receptor polypeptide and that is derived from the same or a different organism.

As used herein, the term “gene” means a segment of DNA that contains all the information for the regulated biosynthesis of an RNA product, including promoters, exons, introns, and other untranslated regions that control expression.

As used herein, the term “genotype” means an unphased 5′ to 3′ sequence of nucleotide pairs found at one or more polymorphic or mutant sites in a locus on a pair of homologous chromosomes in an individual. As used herein, genotype includes a full-genotype and/or a sub-genotype.

As used herein, the term “human sequence antibody” includes antibodies having variable and constant regions (if present) derived from human germline immunoglobulin sequences. The human sequence antibodies of the present technology can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in PCT Publication Nos. WO 01/14424 and WO 00/37504. However, the term “human sequence antibody,” as used herein, is not intended to include antibodies in which CDR sequences derived from the germline of another mammalian species, such as a mouse, have been grafted onto human framework sequences (e.g., humanized antibodies).

As used herein, the term “humanized” forms of non-human (e.g., murine) antibodies are chimeric antibodies which contain minimal sequence derived from non-human immunoglobulin. For the most part, humanized antibodies are human immunoglobulins in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

Amino acid sequence modification(s) of the TRAIL-R2 binding agents described herein are contemplated. For example, it may be desirable to improve the binding affinity and/or other biological properties of the antibody. Amino acid sequence variants of the TRAIL-R2 binding agent are prepared by introducing appropriate nucleotide changes into the antibody nucleic acid, or by peptide synthesis. Such modifications include, for example, deletions from, and/or insertions into and/or substitutions of, residues within the amino acid sequences of the TRAIL-R2 binding agent. Any combination of deletion, insertion, and substitution is made to obtain the antibody of interest, as long as the obtained antibody possesses the desired properties, e.g., biological activity. The modification also includes the change of the pattern of glycosylation of the protein. A useful method for identification of preferred locations for mutagenesis is called “alanine scanning mutagenesis” as described by Cunningham and Wells in Science, 244:1081-1085 (1989). The mutated antibody is then screened for the desired activity. The technology includes antibody variants with one or more amino acid addition, deletion and/or substitution of the amino acid sequence defined by hybridoma CTB006 having having CGMCC Accession Number 1691 provided that the antibody variant possesses the desired properties, e.g., biological activity.

As used herein, the term “hypervariable region” refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

As used herein, the terms “identical” or percent “identity,” when used in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. In some embodiments, identity exists over a region that is at least about 25 amino acids or nucleotides in length; additionally or alternatively, in some embodiments, identity exists over a region that is 50-100 amino acids or nucleotides in length.

An “isolated” or “purified” polypeptide or biologically-active portion thereof is substantially free of cellular material or other contaminating polypeptides from the cell or tissue source from which the TRAIL receptor-binding agent is derived, or substantially free from chemical precursors or other chemicals when chemically synthesized. For example, an isolated TRAIL receptor-binding agent which is an anti-TRAIL receptor antibody would be free of materials that would interfere with diagnostic or therapeutic uses of the agent. Such interfering materials may include enzymes, hormones and other proteinaceous and nonproteinaceous solutes.

As used herein, the phrase “induce cell death” or “capable of inducing cell death” refers to ability of the TRAIL receptor binding agents of the present technology to make a viable cell become nonviable. Cell death and cell viability can be determined by various method in the art such as trypan blue exclusion assay and other cell viability assays. In the present technology, the cell death is specially induced by “apoptosis”, or called “programmed cell death,” which determined by binding of annexin V, fragment of DNA, cell shrinkage, dilation of endoplasmic reticulum, cell fragmentation, and/or formation of membrane vesicles (called apoptotic bodies). Various methods are available for evaluating the cellular events associated with apoptosis. For example, phosphatidyl serine(PS) translocation can be measured by annexin binding; DNA fragmentation can be evaluated through DNA laddering; and nuclear/chromatin condensation along with DNA fragmentation can be evaluated by any increase on hypodiploid cells. A target cell is one which express TRAIL-R2, preferably the cell is a tumor cell, e.g., a breast, colon, ovarian, stomach, endomethial, salivary gland, lung, kidney, thyroid, pancreatic or bladder cell.

As used herein, the term “intact antibody” means an antibody that has at least two heavy (H) chain polypeptides and two light (L) chain polypeptides interconnected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system.

As used herein, the term “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens, cells or tissues infected with pathogens, or, in cases of autoimmunity or pathological inflammation, normal human cells or tissues.

As used herein, the terms “immunologically cross-reactive” and “immunologically-reactive” are used interchangeably to mean an antigen which is specifically reactive with an antibody which was generated using the same (“immunologically-reactive”) or different (“immunologically cross-reactive”) antigen. Generally, the antigen is TRAIL receptor polypeptide, a variant or subsequence thereof.

As used herein, the term “immunologically-reactive conditions” means conditions which allow an antibody, generated to a particular epitope of an antigen, to bind to that epitope to a detectably greater degree than the antibody binds to substantially all other epitopes, generally at least two times above background binding Immunologically-reactive conditions are dependent upon the format of the antibody binding reaction and typically are those utilized in immunoassay protocols. See, Harlow & Lane, Antibodies, A Laboratory Manual (Cold Spring Harbor Publications, New York, 1988) for a description of immunoassay formats and conditions.

As used herein, the term “lymphocyte” means any of the mononuclear, nonphagocytic leukocytes, found in the blood, lymph, and lymphoid tissues, e.g., B and T lymphocytes.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present technology may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example.

As used herein, the term “medical condition” includes, but is not limited to, any condition or disease manifested as one or more physical and/or psychological symptoms for which treatment and/or prevention is desirable, and includes previously and newly identified diseases and other disorders.

As used herein, the term “modulator” includes inhibitors and activators. Inhibitors are agents that, e.g., bind to, partially or totally block stimulation, decrease, prevent, delay activation, inactivate, desensitize, or down regulate the activity of TRAIL receptor polypeptide, e.g., antagonists. Activators are agents that, e.g., bind to, stimulate, increase, open, activate, facilitate, enhance activation, sensitize or up regulate the activity of TRAIL receptor polypeptide, e.g., agonists. Modulators include agents that, e.g., alter the interaction of TRAIL receptor polypeptide with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of a naturally-occurring TRAIL receptor polypeptide, e.g., with altered activity, as well as naturally-occurring and synthetic ligands, antagonists, agonists, small chemical molecules and the like.

As used herein, the term “neutralizing antibody” means an antibody molecule that is able to eliminate or significantly reduce at least one (1) biological function of a TRAIL receptor polypeptide or a TRAIL receptor-like polypeptide.

As used herein, the term “nucleotide pair” means the two nucleotides bound to each other between the two nucleotide strands.

As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration.

As used herein, the term “polyclonal antibody” means a preparation of antibodies derived from at least two (2) different antibody-producing cell lines. The use of this term includes preparations of at least two (2) antibodies that contain antibodies that specifically bind to different epitopes or regions of an antigen. As used herein, the term “polynucleotide” means any RNA or DNA, which may be unmodified or modified RNA or DNA. Polynucleotides include, without limitation, single- and double-stranded DNA, DNA that is a mixture of single- and double-stranded regions, single- and double-stranded RNA, RNA that is mixture of single- and double-stranded regions, and hybrid molecules comprising DNA and RNA that may be single-stranded or, more typically, double-stranded or a mixture of single- and double-stranded regions. In addition, polynucleotide refers to triple-stranded regions comprising RNA or DNA or both RNA and DNA. The term polynucleotide also includes DNAs or RNAs containing one or more modified bases and DNAs or RNAs with backbones modified for stability or for other reasons. In a particular embodiment, the polynucleotide contains polynucleotide sequences from a TRAIL receptor gene.

As used herein, the terms “polypeptide,” “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature. In a particular embodiment, the polypeptide contains polypeptide sequences from a TRAIL receptor protein.

As used herein, the term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the material is derived from a cell so modified. Thus, e.g., recombinant cells express genes that are not found within the native (non-recombinant) form of the cell or express native genes that are otherwise abnormally expressed, under expressed or not expressed at all.

As used herein, the phrase “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule To increase the serum half life of the antibody, one may incorporate a salvage receptor binding epitope into the antibody (especially an antibody fragment) as described in U.S. Pat. No. 5,739,277, for example.

As used herein, the terms “single chain antibodies” or “single chain Fv (scFv)” refer to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). See, e.g., Bird et al., Science 242: 423-426, 1988; and Huston et al., Proc. Natl. Acad. Sci. USA, 85: 5879-5883, 1988). Such single chain antibodies are included by reference to the term “antibody” fragments, and can be prepared by recombinant techniques or enzymatic or chemical cleavage of intact antibodies.

As used herein, the term “small molecule” means a composition that has a molecular weight of less than about 5 kDa and more preferably less than about 2 kDa. Small molecules can be, e.g., nucleic acids, peptides, polypeptides, glycopeptides, peptidomimetics, carbohydrates, lipids, lipopolysaccharides, combinations of these, or other organic or inorganic molecules.

As used herein, the term “specific binding” means the contact between a TRAIL receptor-binding agent and an antigen with a binding affinity of at least 10⁻⁶ M. Preferred binding agents bind with affinities of at least about 10⁻⁷ M, and preferably 10⁻⁸M to 10⁻⁹ M, 10⁻¹⁰ M, 10⁻¹¹ M, or 10⁻¹² M.

As used herein, the phrase “stringent hybridization conditions” means conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as follows: 50% formamide, 5×SSC, and 1% SDS, incubating at 42° C., or, 5×SSC, 1% SDS, incubating at 65° C., with wash in 0.2×SSC, and 0.1% SDS at 65° C.

As used herein, the term “subject” refers to an animal, preferably a mammal, such as a human, but can also be another mammal, e.g., domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substitution” is one type of mutation that is generally known in the art. Exemplary substitution variants have at least one amino acid residue in the TRAIL-R2 binding agent (e.g., antibody) molecule replaced by a different amino acid residue. The sites of greatest interest for substitutional mutagenesis include the hypervariable regions, but FR alterations are also contemplated. “Conservative substitutions” are shown in the Table below under the heading of “preferred substitutions”. If such substitutions result in a change in biological activity, then more substantial changes, denominated “exemplary substitutions” in Table C, or as further described below in reference to amino acid classes, may be introduced and the products screened.

TABLE C
Amino Acid Substitutions

| | Exemplary | Preferred |
|---|---|---|
| Original Residue | Substitutions | Substitutions |
| Ala (A) | val; leu; ile | val |
| Arg (R) | lys; gln; asn | lys |
| Asn (N) | gln; his; asp, lys; arg | gln |
| Asp (D) | glu; asn | glu |
| Cys (C) | ser; ala | ser |
| Gln (Q) | asn; glu | asn |
| Glu (E) | asp; gln | asp |
| Gly (G) | ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | tyr |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

In some embodiments, a substitutional variant involves substituting one or more hypervariable region residues of a parent antibody. A convenient way for generating such substitutional variants involves affinity maturation using phage display. Specifically, several hypervariable region sites (e.g. 6-7 sites) are mutated to generate all possible amino acid substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity). In order to identify candidate hypervariable region sites for modification, alanine scanning mutagenesis can be performed to identify hypervariable region residues contributing significantly to binding TRAIL-R2 receptors. Alternatively, or additionally, it may be beneficial to analyze a crystal structure of the antigen-antibody complex to identify contact points between the antibody and TRAIL-R2 receptors. Such contact residues and neighboring residues are candidates for substitution according to the techniques elaborated herein. Once such variants are generated, the panel of variants is subjected to screening as described herein and antibodies with similar or superior properties in one or more relevant assays may be selected for further development. The present technology includes antibody variants with one or more amino acid substitution(s), especially conservative substitutions, to the hypervariable domains of the immunoglobulin heavy or light chain defined by hybridoma CTB006 having CGMCC Accession Number 1691 provided that the antibody variant possesses the desired properties, e.g., biological activity.

As used herein, the term “target cell” means any cell in a subject (e.g., a human or animal) that can be targeted by the TRAIL receptor-binding agent of the present technology.

As used herein, the term “therapeutic agent” is intended to mean a compound that, when present in an effective amount, produces a desired therapeutic effect on a subject in need thereof.

As used herein, the terms “treating” or “treatment” or “alleviation” refers to therapeutic treatment. A subject is successfully “treated” for a cancer in which cancer cells express TRAIL-R2 if, after receiving a therapeutic amount of a TRAIL-R2 binding agent of the present technology according to the methods disclosed herein, the subject shows observable and/or measurable reduction in or absence of one or more signs and symptoms of the particular disease. For example, for cancer, reduction in the number of cancer cells or absence of the cancer cells; reduction in the tumor size, tumor weight, tumor volume, tumor bearing rate; inhibition (i.e., slow to some extent and preferably stop) of tumor metastasis; inhibition, to some extent, of tumor growth; increase in length of remission, and/or relief to some extent, one or more of the symptoms associated with the specific cancer; reduced morbidity and mortality; increase in body weight, and improvement in quality of life issues. “Prevention” or “preventing” a disease or condition refers to prophylactic or preventative measures, wherein the object is to prevent or slow down (lessen) the targeted pathologic condition or disorder.

As used herein, the term “variable” refers to the fact that certain segments of the variable domains differ extensively in sequence among antibodies. The V domain mediates antigen binding and define specificity of a particular antibody for its particular antigen. However, the variability is not evenly distributed across the amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

### III. Compositions

TRAIL receptor-binding Agents. In one aspect, the present technology provides TRAIL receptor-binding agent compositions (“the binding agent”). In some embodiments, the binding agent of the present technology is an intact antibody directed to a TRAIL receptor polypeptide, homolog or derivative thereof. The binding agents of interest may be ones which bind specifically to TRAIL-R2, but do not “substantively” (or “substantially”) bind other TRAIL receptors such as TRAIL-R1, TRAIL-R3 or TRAIL-R4. That is to say, the antibody of interest may not significantly cross-react with other TRAIL receptors such as TRAIL-R1, TRAIL-R3 or TRAIL-R4. In such embodiments, the extent of binding of the binding agent of the present technology to these proteins will be less than about 10%, preferably, less than 5%, less than 1%, as determined by, e.g., fluorescence activated cell sorting (FACS) analysis, ELISA, chemiluminescent enzyme immunoassay (CLEIA) or radioimmunoprecipitation (RIA).

Binding agents of the present technology can be described or specified in terms of the epitope(s) or portion(s) of a polypeptide of the present technology which are recognized or specifically bound by the binding agent, e.g., a region of the TRAIL-R2 receptor polypeptide that is located on the surface of the polypeptide (e.g., a hydrophilic region). In one embodiment, the present technology provides TRAIL-R2 receptor-binding agents, e.g., antibodies or antibody-related polypeptides directed to a TRAIL-R2 receptor polypeptide (a.k.a., a target polypeptide). The binding agent selectively induces apoptosis of tumor cells in vivo and in vitro, which express TRAIL-R2. In addition, the binding agent has unexpected and a significant, synergistic effect when administered in combination with one or more chemotherapeutic agents. Based on its anti-cancer activity, the TRAIL-R2 binding agent has utility as a reagent for apoptosis signaling research, as well as a therapeutic against cells expressing TRAIL-R2 receptors illustratively including broad classes of cancer cells.

In some embodiments, the technology provides the TRAIL receptor-binding agents summarized in Table D.

TABLE D
Select TRAIL Receptor-Binding Agents

| Binding Agent | Type | Description |
|---|---|---|
| CTB006 | Murine Monoclonal | Murine monospecific antibody |
| | Antibody | directed to TRAIL-R2 receptor. |
| HuCTB006 | Humanized | Monospecific humanized chimeric |
| | Chimeric Antibody | monoclonal antibody directed to |
| | | TRAIL-R2 receptor. |

Deposits of biological materials associated with the TRAIL receptor-binding agents summarized in Table D (above) were made with the China General Microbiological Culture Collection Center (CGMCC), China Committee for Culture Collection of Microorganisms, P.O. Box 2714, Beijing 100080, The People's Republic of China as detailed in Table E below.

TABLE E
Biological Deposits

| Name of | | | Accessing |
|---|---|---|---|
| Depository | Materials | Date | Number |
| CTB006 | Mouse hybridoma | Apr. 11, 2006 | 1691 |
| hCTB006LC | Plasmid DNA of human | Apr. 13, 2007 | 2002 |
| | CTB006 light chain | | |
| hCTB006HC | Plasmid DNA of human | Apr. 13, 2007 | 2003 |
| | CTB006 heavy chain | | |

In another embodiment, the present technology provides methods of elucidating epitopes of TRAIL-R2, which can be used for the generation of an apoptosis-inducing antibody through binding to TRAIL-R2. TNF-related apoptosis-inducing ligand receptor 2, as known as TRAIL receptor 2 or TRAIL-R2, (UniProtKB/Swiss-Prot Database accession no. 014763), has the following sequence: MEQRGQNAPA ASGARKRHGP GPREARGARP GPRVPKTLVL VVAAVLLLVS AESALITQQD LAPQQRAAPQ QKRSSPSEGL CPPGHHISED GRDCISCKYG QDYSTHWNDL LFCLRCTRCD SGEVELSPCT TTRNTVCQCE EGTFREEDSP EMCRKCRTGC PRGMVKVGDC TPWSDIECVH KESGTKHSGE VPAVEETVTS SPGTPASPCS LSGIIIGVTV AAVVLIVAVF VCKSLLWKKV LPYLKGICSG GGGDPERVDR SSQRPGAEDN VLNEIVSILQ PTQVPEQEME VQEPAEPTGV NMLSPGESEH LLEPAEAERS QRRRLLVPAN EGDPTETLRQ CFDDFADLVP FDSWEPLMRK LGLMDNEIKV AKAEAAGHRD TLYTMLIKWV NKTGRDASVH TLLDALETLG ERLAKQKIED HLLSSGKFMY LEGNADSAMS (SEQ ID NO: 15). The binding agents directed against TRAIL-R2 may have a differing variable or CDR region but will have the binding and functional characteristics of the binding agents of the present technology. As a means for targeting antibody production, hydropathy plots showing regions of hydrophilicity and hydrophobicity can be generated by any method well known in the art, including, e.g., the Kyte Doolittle or the Hopp Woods methods, either with or without Fourier transformation (see, e.g., Hopp and Woods, Proc. Nat. Acad. Sci. USA 78: 3824-3828 (1981); Kyte and Doolittle, J. Mol. Biol. 157: 105-142 (1982)). The epitope(s) or polypeptide portion(s) can be specified as described herein, e.g., by N-terminal and C-terminal positions, by size in contiguous amino acid residues. The present technology includes binding agents that specifically bind the TRAIL-R2 receptor.

Binding agents of the present technology can also be described or specified in terms of their cross-reactivity. Binding agents that do not bind any other analog, ortholog, or homolog of the target polypeptide of the present technology are included. Binding agents that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present technology are also included. Further included in the present technology are binding agents which only bind polypeptides encoded by polynucleotides which hybridize to a polynucleotide of the present technology under stringent hybridization conditions (as described herein). Binding agents of the present technology can also be described or specified in terms of their binding affinity. Preferred binding affinities include those with a dissociation constant or K_{d} less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M. In some embodiments, the present technology provides TRAIL receptor binding agents that bind human TRAIL-R2, with a K_{d} value of no higher than 1×10⁸, preferably a K_{d} value no higher than about 1×10⁻⁹.

TRAIL receptor-binding agents within the scope of the present technology include, e.g., but are not limited to, monoclonal, polyclonal, chimeric, humanized, diabody, and human monoclonal and human polyclonal antibodies which specifically bind the target polypeptide, a homolog, derivative or a fragment thereof. As used herein, a “TRAIL receptor-like polypeptide” means a polypeptide that is different from TRAIL receptor polypeptide but which is immunologically-reactive with a TRAIL receptor-binding agent of the present technology. A TRAIL receptor-like polypeptide may be derived from the same organism or a different organism as a TRAIL receptor polypeptide. A TRAIL receptor-like polypeptide may be encoded by the same gene or a different gene as a TRAIL receptor polypeptide. The antibodies useful as binding agents of the present technology include, e.g., but are not limited to, IgG (including IgG₁, IgG₂, IgG₃, and IgG₄), IgA (including IgA₁ and IgA₂), IgD, IgE, or IgM, and IgY.

In another embodiment, the binding agent of the present technology is an antibody-related polypeptide directed to TRAIL receptor polypeptide, homolog or derivative thereof. Typically, the antigen-binding region of a binding agent, e.g., the anti-TRAIL receptor-binding region, will be most critical in specificity and affinity of binding of the binding agent. In some embodiments, the TRAIL receptor-binding agent is an anti-TRAIL receptor polypeptide antibody, such as an anti-TRAIL receptor polypeptide monoclonal antibody, an anti-TRAIL receptor polypeptide chimeric antibody, and an anti-TRAIL receptor polypeptide humanized antibody which have been modified by, e.g., deleting, adding, or substituting portions of the antibody. For example, an anti-TRAIL receptor polypeptide antibody may be modified to increase half-life, e.g., serum half-life, stability or affinity of the antibody.

In some embodiments, selection of antibodies that are specific to a particular domain of a TRAIL receptor polypeptide, e.g., a TRAIL-R2 receptor, is facilitated by generation of hybridomas that bind to the fragment of a TRAIL receptor polypeptide possessing such a domain Thus, in some embodiments, TRAIL receptor-binding agents are antibodies that are specific for a desired domain within a TRAIL receptor polypeptide, and include derivatives, fragments, analogs or homologs thereof.

The present technology further includes antibodies which are anti-idiotypic to the binding agents of the present technology. The binding agents of the present technology can be monospecific, bispecific, trispecific or of greater multispecificity. Multispecific binding agents can be specific for different epitopes of a TRAIL receptor polypeptide of the present technology or can be specific for both a TRAIL receptor polypeptide of the present technology as well as for heterologous compositions, such as a heterologous polypeptide or solid support material. See, e.g., WO 93/17715; WO 92/08802; WO 91/00360; WO 92/05793; Tutt et al., J. Immunol. 147: 60-69 (1991); U.S. Pat. Nos. 5,573,920, 4,474,893, 5,601,819, 4,714,681, 4,925,648; 6,106,835; Kostelny et al., J. Immunol. 148: 1547-1553 (1992). The binding agents of the present technology can be from any animal origin including birds and mammals. In some embodiments, the binding agents are human, murine, rabbit, goat, guinea pig, camel, horse, or chicken.

The binding agents of the present technology are suitable for administration to a subject where it is desirable, e.g., to modulate a TRAIL receptor polypeptide function. Accordingly, it is further an object of the present technology to provide for TRAIL receptor-binding agent compositions that are TRAIL receptor modulators, e.g., functional antagonists or functional agonists of a TRAIL receptor polypeptide. It is also an object of the technology to provide for TRAIL receptor-binding agent compositions that are partial antagonists and partial agonists of a TRAIL receptor polypeptide. Likewise included are neutralizing anti-TRAIL receptor antibodies which bind the TRAIL receptor polypeptide. In some embodiments, the binding agent of the technology will be purified: (1) to greater than 95% by weight of antibody as determined by the Lowry method (Lowry et al., J. Biol. Chem. 193: 265. 1951) and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated binding agent includes the polypeptide in situ within recombinant cells since at least one component of the antibody's natural environment will not be present. Ordinarily, however, a TRAIL receptor-binding agent, e.g., an isolated anti-TRAIL receptor antibody, will be prepared by at least one purification step.

### IV. Combination Therapy

The binding agents of the present technology can be used either alone or in combination with other compositions or active agents. The TRAIL receptor-binding agents of the present technology can further be recombinantly fused to a heterologous polypeptide at the N- or C-terminus or chemically conjugated (including covalently and non-covalently conjugations) to polypeptides or other compositions or active agents. For example, TRAIL receptor-binding agents of the present technology can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

In certain embodiments, the TRAIL receptor-binding agents of the present technology are anti-TRAIL receptor antibodies or anti-TRAIL receptor antibody-related polypeptides that are coupled or conjugated to one or more therapeutic or cytotoxic moieties to yield a TRAIL receptor-binding agent conjugate protein. The TRAIL receptor-binding agent conjugate protein of can be used to modify a given biological response or create a biological response (e.g., to recruit effector cells). The therapeutic moiety is not to be construed as limited to classical chemical therapeutic agents. For example, the therapeutic moiety can be a protein or polypeptide possessing a desired biological activity. Such proteins can include, e.g., an enzymatically active toxin, or active fragment thereof, such as abrin, ricin A, Pseudomonas exotoxin, or diphtheria toxin; a protein such as tumor necrosis factor or interferon-alpha; or, biological response modifiers such as, e.g., lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

In some embodiments, the TRAIL receptor-binging agents of the present technology (e.g., monoclonal antibodies having the same epitope specificity as the mouse-mouse hybridoma CTB006 having CGMCC Accession No. 1691; antibodies comprising heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; antibodies having the heavy chain amino acid sequence set forth in SEQ ID NO: 14 and the light chain amino acid sequence set forth in SEQ ID NO: 12; HuCTB006 antibody), are administered simultaneously, sequentially or separately to a subject, along with a chemotherapeutic agent.

An exemplary and non-limiting list of chemotherapeutic agents is provided herein. Suitable chemotherapeutic drugs include, by way of example but not by way of limitation, vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, therapeutic antibodies, retinoids (such as all-trans retinoic acids or a derivatives thereof); geldanamycin or a derivative thereof (such as 17-AAG), and other standard chemotherapeutic agents well recognized in the art.

In some embodiments, the chemotherapeutic agent includes one or more of adriamycin, colchicine, cyclophosphamide, actinomycin, bleomycin, duanorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., taxol, paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Oxaliplatin, Herceptin®, vinorelbine, Doxil®, capecitabine, Alimta®, Avastin®, Velcade®, Tarceva®, Neulasta®, lapatinib, sorafenib, erlotinib, erbitux, derivatives thereof, chemotherapeutic agents known in the art, and the like. In some embodiments, the chemotherapeutic agent is a composition comprising nanoparticles comprising a thiocolchicine derivative and a carrier protein (such as albumin).

In some embodiments, the chemotherapeutic agent is an antineoplastic agent including, but is not limited to, carboplatin, Navelbine® (vinorelbine), anthracycline (Doxil®), lapatinib (GW57016), Herceptin®, gemcitabine (Gemzar®), capecitabine (Xeloda®), Alimta®, cisplatin, 5-fluorouracil (5-Fu), epirubicin, cyclophosphamide, Avastin®, Velcade®, etc.

Reference to a chemotherapeutic agent herein applies to the chemotherapeutic agent or its derivatives and accordingly the present technology includes either of these embodiments (agent; agent or derivative(s)). “Derivatives” or “analogs” of a chemotherapeutic agent or other chemical moiety include, but are not limited to, compounds that are structurally similar to the chemotherapeutic agent or moiety or are in the same general chemical class as the chemotherapeutic agent or moiety. In some embodiments, the derivative or analog of the chemotherapeutic agent or moiety retains similar chemical and/or physical property (including, for example, functionality) of the chemotherapeutic agent or moiety.

### V. Methods of Preparing Trail Receptor-Binding Agents and Compositions Comprising the Same

General Overview. Initially, a target polypeptide is chosen to which a binding agent of the present technology (e.g., anti-TRAIL receptor antibody) can be raised. Techniques for generating binding agents directed to target polypeptides are well known to those skilled in the art. Examples of such techniques include, e.g., but are not limited to, those involving display libraries, xeno or humab mice, hybridomas, and the like. Target polypeptides within the scope of the present technology include any polypeptide or polypeptide derivative which is capable of exhibiting antigenicity. Examples include, but are not limited to, proteins (e.g., receptors, enzymes, hormones, growth factors), peptides, glycoproteins, lipoproteins, TRAIL receptor polypeptides, and the like. Exemplary target polypeptides also include bacterial, fungal and viral pathogens that cause human disease, such as HIV, hepatitis (A, B, & C), influenza, herpes, Giardia, malaria, Leishmania, Staphylococcus aureus, Pseudomonas aeruginosa. Other target polypeptides are human proteins whose expression levels or compositions have been correlated with human disease or other phenotype. Other targets polypeptides of interest include tumor cell antigens and viral particle antigens.

It should be understood that not only are naturally-occurring antibodies suitable as binding agents for use in accordance with the present disclosure, but recombinantly engineered antibodies and antibody fragments, e.g., antibody-related polypeptides, which are directed to TRAIL receptor polypeptide are also suitable.

Binding agents, e.g., anti-TRAIL receptor antibodies, that can be subjected to the techniques set forth herein include monoclonal and polyclonal antibodies, and antibody fragments such as Fab, Fab′, F(ab′)₂, Fd, scFv, diabodies, antibody light chains, antibody heavy chains and/or antibody fragments. Methods useful for the high yield production of antibody Fv-containing polypeptides, e.g., Fab′ and F(ab′)₂ antibody fragments have been described. See U.S. Pat. No. 5,648,237.

Generally, a binding agent is obtained from an originating species. More particularly, the nucleic acid or amino acid sequence of the variable portion of the light chain, heavy chain or both, of an originating species antibody having specificity for a target polypeptide antigen is obtained. Originating species is any species which was useful to generate the binding agent of the present technology or library of binding agents, e.g., rat, mice, rabbit, chicken, monkey, human, and the like.

In some embodiments, TRAIL receptor-binding agents are anti-TRAIL receptor antibodies. Phage or phagemid display technologies are useful techniques to derive the binding agents of the present technology. Anti-TRAIL receptor antibodies useful in the present technology are “human antibodies,” (e.g., antibodies isolated from a human) or “human sequence antibodies.” Human antibodies can be made by a variety of methods known in the art including phage display methods. See also, U.S. Pat. Nos. 4,444,887, 4,716,111, 5,545,806, and 5,814,318; and WO 98/46645, WO 98/50433, WO 98/24893, WO 98/16654, WO 96/34096, WO 96/33735, and WO 91/10741. Methods useful for the identification of nucleic acid sequences encoding members of multimeric polypeptide complex by screening polyphage particles have been described. Rudert et al., U.S. Pat. No. 6,667,150. Also, recombinant immunoglobulins can be produced. Cabilly, U.S. Pat. No. 4,816,567; Cabilly et al., U.S. Pat. No. 6,331,415 and Queen et al., Proc. Nat'l Acad. Sci. USA 86: 10029-10033, 1989. Techniques for generating and cloning monoclonal antibodies are well known to those skilled in the art. The TRAIL receptor-binding agent of the present technology preferably have a high immunoreactivity, that is, percentages of antibodies molecules that are correctly folded so that they can specifically bind their target antigen. Expression of sequences encoding binding agents, e.g., antibodies of the present technology, can be carried out in E. coli as described below. Such expression usually results in immunoreactivity of at least 80%, 90%, 95% or 99%.

Certain truncations of these proteins or genes perform the regulatory or enzymatic functions of the full sequence protein or gene. For example, the nucleic acid sequences coding therefore can be altered by substitutions, additions, deletions or multimeric expression that provide for functionally equivalent proteins or genes. Due to the degeneracy of nucleic acid coding sequences, other sequences which encode substantially the same amino acid sequences as those of the naturally occurring proteins may be used in the practice of the present technology. These include, but are not limited to, nucleic acid sequences including all or portions of the nucleic acid sequences encoding the above polypeptides, which are altered by the substitution of different codons that encode a functionally equivalent amino acid residue within the sequence, thus producing a silent change. It is appreciated that the nucleotide sequence of an immunoglobulin according to the present technology tolerates sequence homology variations of up to 25% as calculated by standard methods (“Current Methods in Sequence Comparison and Analysis,” Macromolecule Sequencing and Synthesis, Selected Methods and Applications, pp. 127-149, 1998, Alan R. Liss, Inc.) so long as such a variant forms an operative antibody which recognizes TRAIL-R1 and TRAIL-R2. For example, one or more amino acid residues within a polypeptide sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Also included within the scope of the present technology are proteins or fragments or derivatives thereof which are differentially modified during or after translation, e.g., by glycosolation, protolytic cleavage, linkage to an antibody molecule or other cellular ligands, etc. Additionally, an inhibitor encoding nucleic acid sequence can be mutated in vitro or in vivo to create and/or destroy translation, initiation, and/or termination sequences or to create variations in coding regions and/or form new restriction endonuclease sites or destroy pre-existing ones, to facilitate further in vitro modification. Any technique for mutagenesis known in the art can be used, including but not limited to in vitro site directed mutagenesis, J. Biol. Chem. 253:6551, use of Tab linkers (Pharmacia), and the like.

Preparation of Polyclonal Antisera and Immunogens.

Methods of generating antibodies or antibody fragments of the present technology typically include immunizing a subject (generally a non-human subject such as a mouse or rabbit) with the purified TRAIL receptor polypeptide or with a cell expressing the TRAIL receptor polypeptide. Any immunogenic portion of the TRAIL receptor polypeptide can be employed as the immunogen. An appropriate immunogenic preparation can contain, e.g., a recombinantly-expressed TRAIL receptor polypeptide or a chemically-synthesized TRAIL receptor polypeptide. An isolated TRAIL receptor polypeptide, or a portion or fragment thereof, can be used as an immunogen to generate a TRAIL receptor-binding agent that binds to the TRAIL receptor polypeptide, or a portion or fragment using standard techniques for polyclonal and monoclonal antibody preparation. The full-length TRAIL receptor polypeptide can be used or, alternatively, the present technology provides for the use of the TRAIL receptor polypeptide fragments as immunogens. The TRAIL receptor polypeptide comprises at least four amino acid residues of the amino acid sequence shown in SEQ ID NO: 15, and encompasses an epitope of the TRAIL receptor polypeptide such that an antibody raised against the peptide forms a specific immune complex with the TRAIL receptor polypeptide. Preferably, the antigenic peptide comprises at least 5, 8, 10, 15, 20, or 30 amino acid residues. Longer antigenic peptides are sometimes preferable over shorter antigenic peptides, depending on use and according to methods well known to those skilled in the art. Typically, the immunogen will be at least about 8 amino acyl residues in length, and preferably at least about 10 acyl residues in length. Multimers of a given epitope are sometimes more effective than a monomer.

If needed, the immunogenicity of the TRAIL receptor polypeptide (or fragment thereof) can be increased by fusion or conjugation to a hapten such as keyhole limpet hemocyanin (KLH) or ovalbumin (OVA). Many such haptens are known in the art. One can also combine the TRAIL receptor polypeptide with a conventional adjuvant such as Freund's complete or incomplete adjuvant to increase the subject's immune reaction to the polypeptide. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory compounds. These techniques are standard in the art.

For convenience, immune responses are often described in the present technology as being either “primary” or “secondary” immune responses. A primary immune response, which is also described as a “protective” immune response, refers to an immune response produced in an individual as a result of some initial exposure (e.g., the initial “immunization”) to a particular antigen, e.g., a TRAIL receptor polypeptide. Such an immunization can occur, e.g., as the result of some natural exposure to the antigen (e.g., from initial infection by some pathogen that exhibits or presents the antigen) or from antigen presented by cancer cells of some tumor in the individual (e.g., malignant melanoma). Alternatively, the immunization can occur as a result of vaccinating the individual with a vaccine containing the antigen. For example, the vaccine can be a TRAIL receptor vaccine comprising one or more antigens from a TRAIL receptor polypeptide or a TRAIL receptor-like polypeptide.

A primary immune response can become weakened or attenuated over time and can even disappear or at least become so attenuated that it cannot be detected. Accordingly, the present technology also relates to a “secondary” immune response, which is also described here as a “memory immune response.” The term secondary immune response refers to an immune response elicited in an individual after a primary immune response has already been produced.

Thus, a secondary or immune response can be elicited, e.g., to enhance an existing immune response that has become weakened or attenuated, or to recreate a previous immune response that has either disappeared or can no longer be detected. As an example, and not by way of limitation, a secondary immune response can be elicited by re-introducing to the individual an antigen, e.g., a TRAIL receptor polypeptide or a TRAIL receptor-like polypeptide, that elicited the primary immune response (e.g., by re-administrating a vaccine). However, a secondary immune response to an antigen can also be elicited by administrating other agents that can not contain the actual antigen. For example, the present technology provides methods for potentiating a secondary immune response by administrating a TRAIL receptor-binding agent to an individual. In such methods the actual antigen need not necessarily be administered with the TRAIL receptor-binding agent and the composition containing the TRAIL receptor-binding agent need not necessarily contain the antigen. The secondary or memory immune response can be either a humoral (antibody) response or a cellular response. A secondary or memory humoral response occurs upon stimulation of memory B cells that were generated at the first presentation of the antigen. Delayed type hypersensitivity (DTH) reactions are a type of cellular secondary or memory immune response that are mediated by CD4⁺ cells. A first exposure to an antigen primes the immune system and additional exposure(s) results in a DTH.

Following appropriate immunization, the TRAIL receptor-binding agent, e.g., anti-TRAIL receptor polyclonal antibody can be prepared from the subject's serum. If desired, the antibody molecules directed against the TRAIL receptor polypeptide can be isolated from the mammal (e.g., from the blood) and further purified by well known techniques, such as polypeptide A chromatography to obtain the IgG fraction.

Monoclonal Antibody.

In one embodiment of the present technology, the binding agent is an anti-TRAIL receptor monoclonal antibody. In one embodiment of the present technology, the anti-TRAIL receptor monoclonal antibody is a human anti-TRAIL receptor monoclonal antibody. For preparation of monoclonal antibodies directed towards a particular TRAIL receptor polypeptide, or derivatives, fragments, analogs or homologs thereof, any technique that provides for the production of antibody molecules by continuous cell line culture can be utilized. Such techniques include, but are not limited to, the hybridoma technique (see, e.g., Kohler & Milstein, 1975. Nature 256: 495-497); the trioma technique; the human B-cell hybridoma technique (see, e.g., Kozbor, et al., 1983. Immunol. Today 4: 72) and the EBV hybridoma technique to produce human monoclonal antibodies (see, e.g., Cole, et al., 1985. In: MONOCLONAL ANTIBODIES AND CANCER THERAPY, Alan R. Liss, Inc., pp. 77-96). Human monoclonal antibodies can be utilized in the practice of the present technology and can be produced by using human hybridomas (see, e.g., Cote, et al., 1983. Proc Natl Acad Sci USA 80: 2026-2030) or by transforming human B-cells with Epstein Barr Virus in vitro (see, e.g., Cole, et al., 1985. In: MONOCLONAL ANTIBODIES AND CANCER THERAPY, Alan R. Liss, Inc., pp. 77-96). For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—e.g., a bacteriophage coat, or a bacterial cell surface protein—for expression and display of the fusion polypeptides on phage or bacteria. Amplified sequences can then be expressed and further selected or isolated based, e.g., on the affinity of the expressed antibody or fragment thereof for an antigen or epitope present on the TRAIL receptor polypeptide. Alternatively, hybridomas expressing anti-TRAIL receptor monoclonal antibodies can be prepared by immunizing a subject and then isolating hybridomas from the subject's spleen using routine methods. See, e.g., Milstein et al., (Galfre and Milstein, Methods Enzymol (1981) 73: 3-46). Screening the hybridomas using standard methods will produce monoclonal antibodies of varying specificity (i.e., for different epitopes) and affinity. A selected monoclonal antibody with the desired properties, e.g., TRAIL receptor binding, can be used as expressed by the hybridoma, it can be bound to a molecule such as polyethylene glycol (PEG) to alter its properties, or a cDNA encoding it can be isolated, sequenced and manipulated in various ways. Synthetic dendromeric trees can be added a reactive amino acid side chains, e.g., lysine to enhance the immunogenic properties of the TRAIL receptor polypeptide. Also, CPG-dinucleotide technique can be used to enhance the immunogenic properties of the TRAIL receptor polypeptide. Other manipulations include substituting or deleting particular amino acyl residues that contribute to instability of the antibody during storage or after administration to a subject, and affinity maturation techniques to improve affinity of the antibody of the TRAIL receptor polypeptide.

Hybridoma Technique.

In one embodiment, the binding agent of the present technology is an anti-TRAIL receptor monoclonal antibody produced by a hybridoma which includes a B cell obtained from a transgenic non-human animal, e.g., a transgenic mouse, having a genome comprising a human heavy chain transgene and a light chain transgene fused to an immortalized cell. Hybridoma techniques include those known in the art and taught in Harlow et al., Antibodies: A Laboratory Manual Cold Spring Harbor Laboratory, Cold Spring Harbor, N.Y., 349 (1988); Hammerling et al., Monoclonal Antibodies And T-Cell Hybridomas, 563-681 (1981). Other methods for producing hybridomas and monoclonal antibodies are well known to those of skill in the art.

Phage Display Technique.

As noted above, the binding agents of the present technology can be produced through the application of recombinant DNA and phage display technology. For example, binding agents of the technology, e.g., anti-TRAIL receptor antibodies, can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g., human or murine) by selecting directly with antigen, typically antigen bound or captured to a solid surface or bead. Phage used in these methods are typically filamentous phage including fd and M13 with Fab, Fv or disulfide stabilized Fv antibody domains are recombinantly fused to either the phage gene III or gene VIII protein. In addition, methods can be adapted for the construction of Fab expression libraries (see, e.g., Huse, et al., Science 246: 1275-1281, 1989) to allow rapid and effective identification of monoclonal Fab fragments with the desired specificity for a TRAIL receptor polypeptide, e.g., a polypeptide or derivatives, fragments, analogs or homologs thereof. Other examples of phage display methods that can be used to make the binding agents of the present technology include those disclosed in Huston et al., Proc. Natl. Acad. Sci U.S.A., 85: 5879-5883, 1988; Chaudhary et al., Proc. Natl. Acad. Sci U.S.A., 87: 1066-1070, 1990; Brinkman et al., J. Immunol. Methods 182: 41-50, 1995; Ames et al., J. Immunol. Methods 184: 177-186, 1995; Kettleborough et al., Eur. J. Immunol. 24: 952-958, 1994; Persic et al., Gene 187: 9-18, 1997; Burton et al., Advances in Immunology 57: 191-280, 1994; PCT/GB91/01134; WO 90/02809; WO 91/10737; WO 92/01047; WO 92/18619; WO 93/11236; WO 95/15982; WO 95/20401; WO 96/06213; WO 92/01047 (Medical Research Council et al.); WO 97/08320 (Morphosys); WO 92/01047 (CAT/MRC); WO 91/17271 (Affymax); and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743. Methods useful for displaying polypeptides on the surface of bacteriophage particles by attaching the polypeptides via disulfide bonds have been described by Lohning, U.S. Pat. No. 6,753,136. As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869, 1992; and Sawai et al., AJRI 34: 26-34, 1995; and Better et al., Science 240: 1041-1043, 1988.

Generally, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See e.g. Barbas I I I et al., Phage Display, A Laboratory Manual (Cold Spring Harbor Laboratory Press, Cold Spring Harbor, N.Y., 2001). However, other vector formats could be used for this process, such as cloning the antibody fragment library into a lytic phage vector (modified T7 or Lambda Zap systems) for selection and/or screening.

Expression of Recombinant TRAIL Receptor-Binding Agent.

As noted above, the binding agents of the present technology can be produced through the application of recombinant DNA technology. Recombinant polynucleotide constructs encoding a TRAIL receptor-binding agent of the present technology typically include an expression control sequence operably-linked to the coding sequences of anti-TRAIL receptor antibody chains, including naturally-associated or heterologous promoter regions. As such, another aspect of the present technology includes vectors containing one or more nucleic acid sequences encoding a TRAIL receptor-binding agent of the present technology. For recombinant expression of one or more the polypeptides of the technology, the nucleic acid containing all or a portion of the nucleotide sequence encoding the TRAIL receptor-binding agent is inserted into an appropriate cloning vector, or an expression vector (i.e., a vector that contains the necessary elements for the transcription and translation of the inserted polypeptide coding sequence) by recombinant DNA techniques well known in the art and as detailed below. Methods for producing diverse populations of vectors have been described by Lerner et al., U.S. Pat. Nos. 6,291,160; 6,680,192.

In general, expression vectors useful in recombinant DNA techniques are often in the form of plasmids. In the present specification, “plasmid” and “vector” can be used interchangeably as the plasmid is the most commonly used form of vector. However, the present technology is intended to include such other forms of expression vectors that are not technically plasmids, such as viral vectors (e.g., replication defective retroviruses, adenoviruses and adeno-associated viruses), which serve equivalent functions. Such viral vectors permit infection of a subject and expression in that subject of a compound. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences encoding the TRAIL receptor-binding agent, and the collection and purification of the TRAIL receptor-binding agent, e.g., cross-reacting anti-TRAIL receptor antibodies. See, generally, U.S. Application No. 20020199213. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. Vectors can also encode signal peptide, e.g., pectate lyase, useful to direct the secretion of extracellular antibody fragments. See U.S. Pat. No. 5,576,195.

The recombinant expression vectors of the present technology comprise a nucleic acid encoding a compound with TRAIL receptor-binding properties in a form suitable for expression of the nucleic acid in a host cell, which means that the recombinant expression vectors include one or more regulatory sequences, selected on the basis of the host cells to be used for expression that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, e.g., in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of polypeptide desired, etc. Typical regulatory sequences useful as promoters of recombinant polypeptide expression (e.g., TRAIL receptor-binding agents), include, e.g., but are not limited to, 3-phosphoglycerate kinase and other glycolytic enzymes. Inducible yeast promoters include, among others, promoters from alcohol dehydrogenase, isocytochrome C, and enzymes responsible for maltose and galactose utilization. In one embodiment, a polynucleotide encoding a TRAIL receptor-binding agent of the present technology is operably-linked to an ara B promoter and expressible in a host cell. See U.S. Pat. No. 5,028,530. The expression vectors of the present technology can be introduced into host cells to thereby produce polypeptides or peptides, including fusion polypeptides, encoded by nucleic acids as described herein (e.g., TRAIL receptor-binding agents, etc.).

Another aspect of the present technology pertains to TRAIL receptor-binding agent-expressing host cells, which contain a nucleic acid encoding one or more TRAIL receptor-binding agents. The recombinant expression vectors of the present technology can be designed for expression of a TRAIL receptor-binding agent in prokaryotic or eukaryotic cells. For example, a TRAIL receptor-binding agent can be expressed in bacterial cells such as Escherichia coli, insect cells (using baculovirus expression vectors), fungal cells, e.g., yeast, yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, e.g. using T7 promoter regulatory sequences and T7 polymerase. Methods useful for the preparation screening of polypeptides having predetermined property, e.g., TRAIL receptor-binding agents, via expression of stochastically generated polynucleotide sequences has been described. See U.S. Pat. Nos. 5,763,192; 5,723,323; 5,814,476; 5,817,483; 5,824,514; 5,976,862; 6,492,107; 6,569,641.

Expression of polypeptides in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression of recombinant polypeptide; (ii) to increase the solubility of the recombinant polypeptide; and (iii) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding polypeptide, or polypeptide A, respectively, to the target recombinant polypeptide.

Examples of suitable inducible non-fusion E. coli expression vectors include pTrc (Amrann et al., (1988) Gene 69: 301-315) and pET 11d (Studier et al., GENE EXPRESSION TECHNOLOGY: METHODS IN ENZYMOLOGY 185, Academic Press, San Diego, Calif. (1990) 60-89). Methods for targeted assembly of distinct active peptide or protein domains to yield multifunctional polypeptides via polypeptide fusion has been described by Pack et al., U.S. Pat. Nos. 6,294,353; 6,692,935. One strategy to maximize recombinant polypeptide expression, e.g., a TRAIL receptor-binding agent, in E. coli is to express the polypeptide in host bacteria with an impaired capacity to proteolytically cleave the recombinant polypeptide. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in the expression host, e.g., E. coli (see, e.g., Wada, et al., 1992. Nucl. Acids Res. 20: 2111-2118). Such alteration of nucleic acid sequences of the present technology can be carried out by standard DNA synthesis techniques.

In another embodiment, the TRAIL receptor-binding agent expression vector is a yeast expression vector. Examples of vectors for expression in yeast Saccharomyces cerivisae include pYepSecl (Baldari, et al., 1987. EMBO J. 6: 229-234), pMFa (Kurjan and Herskowitz, Cell 30: 933-943, 1982), pJRY88 (Schultz et al., Gene 54: 113-123, 1987), pYES2 (Invitrogen Corporation, San Diego, Calif.), and picZ (InVitrogen Corp, San Diego, Calif.). Alternatively, a TRAIL receptor-binding agent can be expressed in insect cells using baculovirus expression vectors. Baculovirus vectors available for expression of polypeptides, e.g., TRAIL receptor-binding agents, in cultured insect cells (e.g., SF9 cells) include the pAc series (Smith, et al., Mol. Cell. Biol. 3: 2156-2165, 1983) and the pVL series (Lucklow and Summers, 1989. Virology 170: 31-39).

In yet another embodiment, a nucleic acid encoding a TRAIL receptor-binding agent of the present technology is expressed in mammalian cells using a mammalian expression vector. Examples of mammalian expression vectors include, e.g., but are not limited to, pCDM8 (Seed, Nature 329: 840, 1987) and pMT2PC (Kaufman, et al., EMBO J. 6: 187-195, 1987). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells useful for expression of the TRAIL receptor-binding agents of the present technology. See, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., Genes Dev. 1: 268-277, 1987), lymphoid-specific promoters (Calame and Eaton, Adv. Immunol. 43: 235-275, 1988), in particular promoters of T cell receptors (Winoto and Baltimore, EMBO J. 8: 729-733, 1989) and immunoglobulins (Banerji, et al., 1983. Cell 33: 729-740; Queen and Baltimore, Cell 33: 741-748, 1983.), neuron-specific promoters (e.g., the neurofilament promoter; Byrne and Ruddle, Proc. Natl. Acad. Sci. USA 86: 5473-5477, 1989), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, Science 249: 374-379, 1990) and the α-fetoprotein promoter (Campes and Tilghman, Genes Dev. 3: 537-546, 1989).

The present technology further provides a recombinant expression vector comprising a DNA molecule encoding a TRAIL receptor-binding agent of the present technology cloned into the expression vector in an antisense orientation. That is, the DNA molecule is operatively-linked to a regulatory sequence in a manner that allows for expression (by transcription of the DNA molecule) of an RNA molecule that is antisense to a TRAIL receptor-binding agent mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes. See, e.g., Weintraub, et al., “Antisense RNA as a molecular tool for genetic analysis,” Reviews-Trends in Genetics, Vol. 1(1) 1986.

Another aspect of the present technology pertains to host cells into which a recombinant expression vector has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, a TRAIL receptor-binding agent can be expressed in bacterial cells such as E. coli, insect cells, yeast or mammalian cells. Mammalian cells are a preferred host for expressing nucleotide segments encoding immunoglobulins or fragments thereof. See Winnacker, From Genes To Clones, (VCH Publishers, N Y, 1987). A number of suitable host cell lines capable of secreting intact heterologous proteins have been developed in the art, and include Chinese hamster ovary (CHO) cell lines, various COS cell lines, HeLa cells, L cells and myeloma cell lines. Preferably, the cells are nonhuman. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Queen et al., Immunol. Rev. 89: 49, 1986. Preferred expression control sequences are promoters derived from endogenous genes, cytomegalovirus, SV40, adenovirus, bovine papillomavirus, and the like. Co et al., J Immunol. 148: 1149, 1992. Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation, biolistics or viral-based transfection can be used for other cellular hosts. Other methods used to transform mammalian cells include the use of polybrene, protoplast fusion, liposomes, electroporation, and microinjection (see generally, Sambrook et al., Molecular Cloning). Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals. The vectors containing the DNA segments of interest can be transferred into the host cell by well known methods, depending on the type of cellular host.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the TRAIL receptor-binding agent or can be introduced on a separate vector. Cells stably transfected with the introduced nucleic acid can be identified by drug selection (e.g., cells that have incorporated the selectable marker gene will survive, while the other cells die).

A host cell that includes a TRAIL receptor-binding agent of the present technology, such as a prokaryotic or eukaryotic host cell in culture, can be used to produce (i.e., express) recombinant TRAIL receptor-binding agent. In one embodiment, the method comprises culturing the host cell (into which a recombinant expression vector encoding the TRAIL receptor-binding agent has been introduced) in a suitable medium such that the TRAIL receptor-binding agent is produced. In another embodiment, the method further comprises the step of isolating the TRAIL receptor-binding agent from the medium or the host cell. Once expressed, collections of the TRAIL receptor-binding agents, e.g., the anti-TRAIL receptor antibodies or the anti-TRAIL receptor antibody-related polypeptides are purified from culture media and host cells. The TRAIL receptor-binding agents can be purified according to standard procedures of the art, including HPLC purification, column chromatography, gel electrophoresis and the like. In one embodiment, the TRAIL receptor-binding agent is produced in a host organism by the method of Boss et al., U.S. Pat. No. 4,816,397. Usually, anti-TRAIL receptor antibody chains are expressed with signal sequences and are thus released to the culture media. However, if the anti-TRAIL receptor antibody chains are not naturally secreted by host cells, the anti-TRAIL receptor antibody chains can be released by treatment with mild detergent. Purification of recombinant polypeptides is well known in the art and include ammonium sulfate precipitation, affinity chromatography purification technique, column chromatography, ion exchange purification technique, gel electrophoresis and the like (see generally Scopes, Protein Purification (Springer-Verlag, N.Y., 1982).

Polynucleotides encoding TRAIL receptor-binding agents, e.g., the anti-TRAIL receptor antibody coding sequences, can be incorporated in transgenes for introduction into the genome of a transgenic animal and subsequent expression in the milk of the transgenic animal See, e.g., U.S. Pat. Nos. 5,741,957, 5,304,489, and 5,849,992. Suitable transgenes include coding sequences for light and/or heavy chains in operable linkage with a promoter and enhancer from a mammary gland specific gene, such as casein or β-lactoglobulin. For production of transgenic animals, transgenes can be microinjected into fertilized oocytes, or can be incorporated into the genome of embryonic stem cells, and the nuclei of such cells transferred into enucleated oocytes.

Single Chain Antibodies.

In one embodiment, the binding agent of the present technology is a single chain anti-TRAIL receptor antibody. According to the present technology, techniques can be adapted for the production of single-chain antibodies specific to a TRAIL receptor polypeptide (see, e.g., U.S. Pat. No. 4,946,778). Examples of techniques which can be used to produce single-chain Fvs and antibodies of the present technology include those described in U.S. Pat. Nos. 4,946,778 and 5,258,498; Huston et al., Methods in Enzymology, 203: 46-88, 1991; Shu, L. et al., Proc. Natl. Acad. Sci. USA, 90: 7995-7999, 1993; and Skerra et al., Science 240: 1038-1040, 1988.

Chimeric and Humanized Antibodies.

In one embodiment, the binding agent of the present technology is a chimeric anti-TRAIL receptor antibody. In one embodiment, the binding agent of the present technology is a humanized anti-TRAIL receptor antibody. In one embodiment of the present technology, the donor and acceptor antibodies are monoclonal antibodies from different species. For example, the acceptor antibody is a human antibody (to minimize its antigenicity in a human), in which case the resulting CDR-grafted antibody is termed a “humanized” antibody.

Recombinant anti-TRAIL receptor antibodies, such as chimeric and humanized monoclonal antibodies, comprising both human and non-human portions, can be made using standard recombinant DNA techniques, and are within the scope of the present technology. For some uses, including in vivo use of the binding agent of the present technology in humans as well as use of these agents in vitro detection assays, it is preferable to use chimeric, humanized, or human anti-TRAIL receptor antibodies. Such chimeric and humanized monoclonal antibodies can be produced by recombinant DNA techniques known in the art. Such useful methods include, e.g., but are not limited to, methods described in International Application No. PCT/US86/02269; U.S. Pat. No. 5,225,539; European Patent No. 184187, European Patent No. 171496; European Patent No. 173494; PCT International Publication No. WO 86/01533; U.S. Pat. Nos. 4,816,567; 5,225,539; European Patent No. 125023; Better, et al., 1988. Science 240: 1041-1043; Liu, et al., 1987. Proc. Natl. Acad. Sci. USA 84: 3439-3443; Liu, et al., 1987. J. Immunol. 139: 3521-3526; Sun, et al., 1987. Proc. Natl. Acad. Sci. USA 84: 214-218; Nishimura, et al., 1987. Cancer Res. 47: 999-1005; Wood, et al., 1985. Nature 314: 446-449; Shaw, et al., 1988. J. Natl. Cancer Inst. 80: 1553-1559); Morrison (1985) Science 229: 1202-1207; Oi, et al. (1986) BioTechniques 4: 214; Jones, et al., 1986. Nature 321: 552-525; Verhoeyan, et al., 1988. Science 239: 1534; Morrison, Science 229: 1202, 1985; Oi et al., BioTechniques 4: 214, 1986; Gillies et al., J. Immunol. Methods, 125: 191-202, 1989; U.S. Pat. No. 5,807,715; and Beidler, et al., 1988. J. Immunol. 141: 4053-4060. For example, antibodies can be humanized using a variety of techniques including CDR-grafting (EP 0 239 400; WO 91/09967; U.S. Pat. Nos. 5,530,101; 5,585,089; 5,859,205; 6,248,516; EP460167), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). In one embodiment, a cDNA encoding a murine anti-TRAIL receptor monoclonal antibody is digested with a restriction enzyme selected specifically to remove the sequence encoding the Fc constant region, and the equivalent portion of a cDNA encoding a human Fc constant region is substituted (see Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 125,023; Better et al. (1988) Science 240: 1041-1043; Liu et al. (1987) Proc Natl Acad Sci USA 84: 3439-3443; Liu et al. (1987) J Immunol 139: 3521-3526; Sun et al. (1987) Proc Natl Acad Sci USA 84: 214-218; Nishimura et al. (1987) Cancer Res 47: 999-1005; Wood et al. (1985) Nature 314: 446-449; and Shaw et al. (1988) J Natl Cancer Inst 80: 1553-1559); U.S. Pat. No. 6,180,370; U.S. Pat. Nos. 6,300,064; 6,696,248; 6,706,484; 6,828,422.

In one embodiment, the present technology allows the construction of humanized anti-TRAIL receptor antibodies that are unlikely to induce a human anti-mouse antibody (hereinafter referred to as “HAMA”) response, while still having an effective antibody effector function. As used herein, the terms “human” and “humanized”, in relation to antibodies, relate to any antibody which is expected to elicit a therapeutically tolerable weak immunogenic response in a human subject. In one embodiment, the present technology provides for a humanized TRAIL-R1 and/or TRAIL-R2 dual-specific antibody, CTB003 or hCTB003 heavy and light chain immunoglobulins.

CDR Antibodies.

In one embodiment, the binding agent of the present technology is an anti-TRAIL receptor CDR antibody. Generally the donor and acceptor antibodies used to generate the anti-TRAIL receptor CDR antibody are monoclonal antibodies from different species; typically the acceptor antibody is a human antibody (to minimize its antigenicity in a human), in which case the resulting CDR-grafted antibody is termed a “humanized” antibody. The graft may be of a single CDR (or even a portion of a single CDR) within a single V_{H} or V_{L} of the acceptor antibody, or can be of multiple CDRs (or portions thereof) within one or both of the V_{H} and V_{L}. Frequently all three CDRs in all variable domains of the acceptor antibody will be replaced with the corresponding donor CDRs, though one need replace only as many as necessary to permit adequate binding of the resulting CDR-grafted antibody to MetAp3. Methods for generating CDR-grafted and humanized antibodies are taught by Queen et al. U.S. Pat. No. 5,585,089, U.S. Pat. No. 5,693,761; U.S. Pat. No. 5,693,762; and Winter U.S. Pat. No. 5,225,539; and EP 0682040. Methods useful to prepare V_{H} and V_{L} polypeptides are taught by Winter et al., U.S. Pat. Nos. 4,816,397; 6,291,158; 6,291,159; 6,291,161; 6,545,142; EP 0368684; EP0451216; EP0120694.

After selecting suitable framework region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) can be produced by oligonucleotide synthesis and/or PCR. The nucleic acid encoding CDR regions can also be isolated from the originating species antibodies using suitable restriction enzymes and ligated into the target species framework by ligating with suitable ligation enzymes. Alternatively, the framework regions of the variable chains of the originating species antibody can be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

This process typically does not alter the acceptor antibody's FRs flanking the grafted CDRs. However, one skilled in the art can sometimes improve antigen binding affinity of the resulting anti-TRAIL receptor CDR grafted antibody by replacing certain residues of a given FR to make the FR more similar to the corresponding FR of the donor antibody. Preferred locations of the substitutions include amino acid residues adjacent to the CDR, or which are capable of interacting with a CDR (see, e.g., U.S. Pat. No. 5,585,089, especially columns 12-16). Or one skilled in the art can start with the donor FR and modify it to be more similar to the acceptor FR or a human consensus FR. Techniques for making these modifications are known in the art. Particularly if the resulting FR fits a human consensus FR for that position, or is at least 90% or more identical to such a consensus FR, doing so may not increase the antigenicity of the resulting modified anti-TRAIL receptor CDR antibody significantly compared to the same antibody with a fully human FR.

Fusion Proteins.

In one embodiment, the binding agent of the present technology is a fusion protein. The TRAIL receptor-binding agents, when fused to a second protein, can be used as an antigenic tag. Examples of domains that can be fused to polypeptides include not only heterologous signal sequences, but also other heterologous functional regions. The fusion does not necessarily need to be direct, but can occur through linker sequences. Moreover, fusion proteins of the present technology can also be engineered to improve characteristics of the TRAIL receptor-binding agent. For instance, a region of additional amino acids, particularly charged amino acids, can be added to the N-terminus of the TRAIL receptor-binding agent to improve stability and persistence during purification from the host cell or subsequent handling and storage. Also, peptide moieties can be added to the TRAIL receptor-binding agent to facilitate purification. Such regions can be removed prior to final preparation of the TRAIL receptor-binding agent. The addition of peptide moieties to facilitate handling of polypeptides are familiar and routine techniques in the art. The TRAIL receptor-binding agent can be fused to marker sequences, such as a peptide which facilitates purification of the fused polypeptide. In some embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824, 1989, for instance, hexa-histidine provides for convenient purification of the fusion protein. Another peptide tag useful for purification, the “HA” tag, corresponds to an epitope derived from the influenza hemagglutinin protein. Wilson et al., Cell 37: 767, 1984.

Thus, any of these above fusions can be engineered using the polynucleotides or the polypeptides of the present technology. Also, the fusion protein can show an increased half-life in vivo.

Fusion proteins having disulfide-linked dimeric structures (due to the IgG) can be more efficient in binding and neutralizing other molecules, than the monomeric secreted protein or protein fragment alone. Fountoulakis et al., J. Biochem. 270: 3958-3964, 1995.

Similarly, EP-A-0 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, e.g., improved pharmacokinetic properties. See EP-A 0232 262. Alternatively, deleting the Fc part after the fusion protein has been expressed, detected, and purified, would be desired. For example, the Fc portion can hinder therapy and diagnosis if the fusion protein is used as an antigen for immunizations. In drug discovery, e.g., human proteins, such as hIL-5, have been fused with Fc portions for the purpose of high-throughput screening assays to identify antagonists of hIL-5. Bennett et al., J. Molecular Recognition 8: 52-58, 1995; Johanson et al., J. Biol. Chem., 270: 9459-9471, 1995.

De-Immunization of Therapeutic Proteins by T Cell Epitope Modification.

Many therapeutic proteins in clinical use have been shown to elicit unwanted antibody responses, which in some cases have been linked to adverse events. In one embodiment of the present technology, recombinant anti-TRAIL receptor antibodies, TRAIL receptor polypeptides or TRAIL receptor-binding agent are rendered non-immunogenic, or less immunogenic, to a given species by identifying in their amino acid sequences one or more potential epitopes for T-cells of the given species and modifying the amino acid sequence to eliminate at least one of the T-cell epitopes. This eliminates or reduces the immunogenicity of the polypeptide or protein when exposed to the immune system of the given species. Monoclonal antibodies and other immunoglobulin-like molecules can particularly benefit from being de-immunized in this way—for example, mouse-derived immunoglobulins can be de-immunized for human therapeutic use. Methods for de-immunizing a polypeptide or protein in the art. See, e.g., Carr, et al. US Pat. Application 20030153043; and De Groot, et al., AIDS Res. and Human Retroviruses 13: 539-541 (1997); Schafer, et al., Vaccine 16: 1880-1884 (1998); De Groot, et al., Dev. Biol. 112: 71-80 (2003); De Groot, et al., Vaccine 19: 4385-4395 (2001); Reijonen and Kwok Methods 29: 282-288; Novak, et al., J. Immunology 166: 6665-6670 (2001).

TRAIL receptor-Binding Agent Conjugate Protein.

As noted above, in certain embodiments, the TRAIL receptor-binding agent of the present technology are anti-TRAIL receptor antibodies coupled or conjugated to one or more therapeutic or cytotoxic moieties to yield a TRAIL receptor-binding agent conjugate protein. Optionally, the TRAIL receptor-binding agents are useful as TRAIL receptor-binding agent-cytotoxin conjugate molecules, as exemplified by the administration for treatment of neoplastic disease.

In one embodiment, TRAIL receptor-binding agents of the present technology are prepared using genomic DNA or ESTs encoding candidate binding agents as part of fusion proteins which form inclusion bodies upon expression in host cells. Methods useful to prepare genomic DNA or ESTs encoding candidate binding agents as part of fusion proteins which form inclusion bodies upon expression in host cells have been described. See U.S. Pat. No. 6,653,068; U.S.S.N. 20040157291. For example, the inclusion bodies are useful to generate binding partners, e.g., TRAIL receptor-binding agents, which bind specifically to the target (poly)peptide.

As an alternative coupling method, a moiety can be coupled to the TRAIL receptor-binding agents of the present technology, e.g., through an oxidized carbohydrate group at a glycosylation site, as described in U.S. Pat. Nos. 5,057,313 and 5,156,840. Yet another alternative method of coupling a TRAIL receptor-binding agent to a moiety is by the use of a non-covalent binding pair, such as streptavidin/biotin, or avidin/biotin. In these embodiments, one member of the pair is covalently coupled to the TRAIL receptor-binding agent and the other member of the binding pair is covalently coupled to the moiety.

Suitable linkage chemistries include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups can be designed into recombinant immunoglobulin molecules. It will be evident to those skilled in the art that a variety of bifunctional or polyfunctional reagents, both homo- and hetero-functional (such as those described in the catalogue of the Pierce Chemical Co., Rockford, Ill.), can be employed as a linker group. Coupling can be affected, e.g., through amino groups, carboxyl groups, sulfhydryl groups or oxidized carbohydrate residues (see, e.g., U.S. Pat. No. 4,671,958).

Cleavable linkers.

Where a cytotoxic or therapeutic moiety is more potent when free from the TRAIL receptor-binding agent portion of the immunoconjugates of the present technology, it can be desirable to use a linker group which is cleavable during or upon internalization into a cell, or which is gradually cleavable over time in the extracellular environment. A number of different cleavable linker groups have been described. Examples of the intracellular release of a cytotoxic moiety from these linker groups include, e.g., but are not limited to, cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and acid-catalyzed hydrolysis (e.g., U.S. Pat. No. 4,569,789).

In one embodiment, the TRAIL receptor-binding agent is coupled to more than one therapeutic, cytotoxic and/or imaging moiety. By poly-derivatizing the TRAIL receptor-binding agent, several cytotoxic strategies can be simultaneously implemented, a TRAIL receptor-binding agent can be made useful as a contrasting agent for several visualization techniques, or a therapeutic antibody can be labeled for tracking by a visualization technique. In one embodiment, multiple molecules of a cytotoxic moiety are coupled to one TRAIL receptor-binding agent. In one embodiment, the TRAIL receptor-binding agent is coupled to a mixture of at least two moieties selected from the group consisting of: a cytotoxic moiety; therapeutic moiety; and labelling/imaging moiety. That is, more than one type of moiety can be coupled to one TRAIL receptor-binding agent. For instance, a therapeutic moiety, such as a polynucleotide or antisense sequence, can be conjugated to a TRAIL receptor-binding agent in conjunction with a chemotoxic or radiotoxic moiety, to increase the effectiveness of the chemo- or radiotoxic therapy, as well as lowering the required dosage necessary to obtain the desired therapeutic effect. Regardless of the particular embodiment, immunoconjugates with more than one moiety can be prepared in a variety of ways. For example, more than one moiety can be coupled directly to a TRAIL receptor-binding agent, or linkers that provide multiple sites for attachment (e.g., dendrimers) can be used. Alternatively, a carrier with the capacity to hold more than one cytotoxic moiety can be used.

As explained above, a TRAIL receptor-binding agent can bear the moiety(ies) in a variety of ways, including covalent bonding either directly or via a linker group, and non-covalent associations. In one embodiment, the TRAIL receptor-binding coupled protein can be combined with encapsulation carriers. This is especially useful in chemotoxic therapeutic embodiments, as they can allow the therapeutic compositions to gradually release a TRAIL receptor-binding agent chemotoxic moiety over time while concentrating it in the vicinity of the target cells.

TRAIL receptor-Binding Agent Conjugated with Radionuclides.

In one embodiment, the TRAIL receptor-binding agent of the present technology is coupled with a cytotoxic moiety which is a radionuclide. Preferred radionuclides for use as cytotoxic moieties are radionuclides which are suitable for pharmacological administration. Such radionuclides include ¹²³I, ¹²⁵I, ¹³¹I, ⁹⁰Y, ²¹¹At, ⁶⁷Cu, ¹⁸⁶Re, ¹⁸⁸Re, ²¹²Pb and ²¹²Bi. Iodine and astatine isotopes are more preferred radionuclides for use in the therapeutic compositions of the present technology, as a large body of literature has been accumulated regarding their use. ¹³¹I is particularly preferred, as are other β-radiation emitting nuclides, which have an effective range of several millimeters. ¹²³I, ¹²⁵I, ¹³¹I, or ²¹¹At can be conjugated to the TRAIL receptor-binding agent for use in the compositions and methods utilizing any of several known conjugation reagents, including lodogen, N-succinimidyl 3-[²¹¹At]astatobenzoate, N-succinimidyl 3-[¹³¹I]iodobenzoate (SIB), and, N-succinimidyl 5-[¹³¹I]iodob-3-pyridinecarboxylate (SIPC). Any iodine isotope can be utilized in the recited iodo-reagents. Other radionuclides can be conjugated to the TRAIL receptor-binding agent by suitable chelation agents known to those of skill in the nuclear medicine arts.

In general, therapeutic moeities can be conjugated to the TRAIL receptor-binding agent of the present technology, e.g., by any suitable technique, with appropriate consideration of the need for pharmokinetic stability and reduced overall toxicity to the subject. A therapeutic, cytotoxic, or labelling/imaging agent (i.e., a “moiety”) can be coupled to a suitable TRAIL receptor-binding agent either directly or indirectly (e.g., via a linker group). A direct reaction between a moiety and a TRAIL receptor-binding agent is possible when each possesses a functional group capable of reacting with the other. For example, a nucleophilic group, such as an amino or sulfhydryl group, can be capable of reacting with a carbonyl-containing group, such as an anhydride or an acid halide, or with an alkyl group containing a good leaving group (e.g., a halide). Alternatively, a suitable chemical linker group can be used. A linker group can function as a spacer to distance the TRAIL receptor-binding agent from a moiety in order to avoid interference with binding capabilities. A linker group can also serve to increase the chemical reactivity of a substituent on a moiety or a TRAIL receptor-binding agent, and thus increase the coupling efficiency. An increase in chemical reactivity can also facilitate the use of moieties, or functional groups on moieties, which otherwise would not be possible.

Chemotoxic Moieties.

In one embodiment, the TRAIL receptor-binding agent of the present technology is coupled with a chemotoxic moiety. Chemotoxic agents include, but are not limited to, small-molecule drugs such as methotrexate, and pyrimidine and purine analogs. Chemotoxin differentiation inducers include phorbol esters and butyric acid. Chemotoxic moieties can be directly conjugated to the TRAIL receptor-binding agent. In one embodiment, the TRAIL receptor-binding agent of the present technology is coupled to a cytotoxic moiety via a chemical linker. In another embodiment, a moiety is encapsulated in a carrier, which is, in turn, is coupled to the TRAIL receptor-binding agent of the present technology.

Protein Toxins.

In one embodiment, the TRAIL receptor-binding agent of the present technology is coupled with a protein toxin moiety. Preferred toxin proteins for use as cytotoxic moieties, include, e.g., but are not limited to, Actinomycetes or Streptomyces antibiotics, duocarmycin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin didne, mitoxantrone, mithramycin, actinomycin D, 1-dehydrotestosterone, glucocorticoids, procaine, tetracaine, lidocaine, propranolol, and puromycin and analogs or homologs thereof. Preferred toxin proteins for use as cytotoxic moieties further include ricin, abrin, diphtheria toxin, cholera toxin, gelonin, Pseudomonas exotoxin, Shigella toxin, pokeweed antiviral protein, and other toxin proteins known in the medicinal biochemistry arts. As these toxin agents can elicit undesirable immune responses in the subject, especially if injected intravascularly, it is preferred that they be encapsulated in a carrier for coupling to the TRAIL receptor-binding agents, e.g., the anti-TRAIL receptor antibody and the antibody-related polypeptides.

Enzymatically-Active Toxins.

In one embodiment, the TRAIL receptor-binding agent of the present technology is coupled with an enzymatically active toxin. The enzymatically active toxin can be of bacterial or plant origin, or an enzymatically active fragment (“A chain”) of such a toxin. Enzymatically active toxins and fragments thereof useful in the present technology are diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Sapaonaria officinalis inhibitor, gelonin, mitogellin, restrictocin, phenomycin, and enomycin. Conjugates of the TRAIL receptor-binding agent of the present technology with cytotoxic moieties are made using a variety of bifunctional protein coupling agents. Examples of such reagents are SPDP, IT, bifunctional derivatives of imidoesters such a dimethyl adipimidate HCl, active esters such as disuccinimidyl suberate, aldehydes such as glutaraldehyde, bis-azido compounds such as bis (p-azidobenzoyl) hexanediamine, bis-diazonium derivatives such as bis-(p-diazoniumbenzoyl)-ethylenediamine, diisocyanates such as tolylene 2,6-diisocyanate, and bis-active fluorine compounds such as 1,5-difluoro-2,4-dinitrobenzene. The lysing portion of a toxin can be joined to the Fab fragment of antibodies, e.g., the TRAIL receptor-binding agent.

Therapeutic Moieties.

In one embodiment, the TRAIL receptor-binding agent of the present technology is coupled with a therapeutic moiety. A therapeutic moiety includes, e.g., but is not limited to, anti-metabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), doxorubicin (adriamycin), cisplatin, bleomycin sulfate, carmustine, chlorambucil, cyclophosphamide hydroxyurea or ricin A, and anti-mitotic agents (e.g., vincristine and vinblastine).

Techniques for conjugating such therapeutic moiety to a TRAIL receptor-binding agent of the present technology are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62: 119-58 (1982).

Labeled TRAIL Receptor-Binding Agent.

In one embodiment, the TRAIL receptor-binding agent of the present technology is coupled with a label moiety, i.e., detectable group. The particular label or detectable group conjugated to the TRAIL receptor-binding agent is not a critical aspect of the present technology, so long as it does not significantly interfere with the specific binding of the TRAIL receptor-binding agent of the present technology to the TRAIL receptor polypeptide or the TRAIL receptor-like polypeptide. The detectable group can be any material having a detectable physical or chemical property. Such detectable labels have been well-developed in the field of immunoassays and imaging, in general, most any label useful in such methods can be applied to the present technology. Thus, a label is any composition detectable by spectroscopic, photochemical, biochemical, immunochemical, electrical, optical or chemical means. Useful labels include magnetic beads (e.g., Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹I, ¹¹²In, ⁹⁹mTc), other imaging agents such as microbubbles (for ultrasound imaging), ¹⁸F, ¹¹C, ¹⁵O, (for Positron emission tomography), ^{99m}TC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g., polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6^{th} Ed., Molecular Probes, Inc., Eugene Oreg.).

The label can be coupled directly or indirectly to the desired component of an assay according to methods well known in the art. As indicated above, a wide variety of labels can be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, e.g., biotin, thyroxine, and cortisol, it can be used in conjunction with the labeled, naturally-occurring anti-ligands. Alternatively, any haptenic or antigenic compound can be used in combination with an antibody, e.g., an anti-TRAIL receptor antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds useful as labelling moieties, include, but are not limited to, e.g., fluorescein and its derivatives, rhodamine and its derivatives, dansyl, umbelliferone, and the like. Chemiluminescent compounds useful as labelling moieties, include, but are not limited to, e.g., luciferin, and 2,3-dihydrophthalazinediones, e.g., luminol. For a review of various labeling or signal-producing systems which can be used, see, U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally simple colorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies, e.g., the anti-TRAIL receptor antibodies. In this case, antigen-coated particles are agglutinated by samples comprising the target antibodies. In this format, none of the components need be labeled and the presence of the target antibody is detected by simple visual inspection.

Formulations of Pharmaceutical Compositions.

The TRAIL receptor-binding agent of the present technology can be incorporated into pharmaceutical compositions suitable for administration. The pharmaceutical compositions generally comprise at least one TRAIL receptor-binding agent and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the antibody compositions (see, e.g., Remington's Pharmaceutical Sciences, Mack Publishing Co., Easton, Pa. 18^{th} ed., 1990). The pharmaceutical compositions are generally formulated as sterile, substantially isotonic and in full compliance with all Good Manufacturing Practice (GMP) regulations of the U.S. Food and Drug Administration.

The terms “pharmaceutically-acceptable,” “physiologically-tolerable,” and grammatical variations thereof, as they refer to compositions, carriers, diluents and reagents, are used interchangeably and represent that the materials are capable of administration to or upon a subject without the production of undesirable physiological effects to a degree that would prohibit administration of the composition. For example, “pharmaceutically-acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. “Pharmaceutically-acceptable salts and esters” means salts and esters that are pharmaceutically-acceptable and have the desired pharmacological properties. Such salts include salts that can be formed where acidic protons present in the TRAIL receptor-binding agent are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N-methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically-acceptable esters include esters formed from carboxy, sulfonyloxy, and phosphonoxy groups present in the TRAIL receptor-binding agent, e.g., C₁₋₆ alkyl esters. When there are two acidic groups present, a pharmaceutically-acceptable salt or ester can be a mono-acid-mono-salt or ester or a di-salt or ester; and similarly where there are more than two acidic groups present, some or all of such groups can be salified or esterified. The TRAIL receptor-binding agent named in this technology can be present in unsalified or unesterified form, or in salified and/or esterified form, and the naming of such TRAIL receptor-binding agent is intended to include both the original (unsalified and unesterified) compound and its pharmaceutically-acceptable salts and esters. Also, certain TRAIL receptor-binding agent named in this technology can be present in more than one stereoisomeric form, and the naming of such TRAIL receptor-binding agent is intended to include all single stereoisomers and all mixtures (whether racemic or otherwise) of such stereoisomers. A person of ordinary skill in the art, would have no difficulty determining the appropriate timing, sequence and dosages of administration for particular drugs and compositions of the present technology.

Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the TRAIL receptor-binding agent, use thereof in the compositions is contemplated. Supplementary active compounds can also be incorporated into the compositions.

A pharmaceutical composition of the present technology is formulated to be compatible with its intended route of administration. The TRAIL receptor-binding agent compositions of the present technology can be administered by parenteral, topical, intravenous, oral, subcutaneous, intraarterial, intradermal, transdermal, rectal, intracranial, intraperitoneal, intranasal; intramuscular route or as inhalants. The most typical route of administration of an immunogenic agent, e.g., the TRAIL receptor polypeptide, is subcutaneous although other routes can be equally effective. The next most common route is intramuscular injection. This type of injection is most typically performed in the arm or leg muscles. In some methods, agents are injected directly into a particular tissue where deposits have accumulated, e.g. intracranial injection. Intramuscular injection on intravenous infusion are preferred for administration of the TRAIL receptor-binding agent, e.g., an anti-TRAIL receptor antibody. In some methods, particular TRAIL receptor-binding agents are injected directly into the cranium. In some methods, the TRAIL receptor-binding agents are administered as a sustained release composition or device, such as a Medipad™ device.

The TRAIL receptor-binding agent of the present technology can optionally be administered in combination with other agents that are at least partly effective in treating various diseases including various TRAIL receptor-related diseases. In the case of administration into the central nervous system of a subject, the TRAIL receptor-binding agent can also be administered in conjunction with other agents that increase passage of the agents across the blood-brain barrier.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as manitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating the TRAIL receptor-binding agent in the required amount in an appropriate solvent with one or a combination of ingredients enumerated above, as required, followed by filtered sterilization. Generally, dispersions are prepared by incorporating the binding agent into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The agents of the present technology can be administered in the form of a depot injection or implant preparation which can be formulated in such a manner as to permit a sustained or pulsatile release of the active ingredient.

Oral compositions generally include an inert diluent or an edible carrier. They can be enclosed in gelatin capsules or compressed into tablets. For the purpose of oral therapeutic administration, the binding agent can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the TRAIL receptor-binding agent are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of nasal sprays or suppositories. For transdermal administration, the TRAIL receptor-binding agent is formulated into ointments, salves, gels, or creams as generally known in the art.

The TRAIL receptor-binding agent can also be prepared as pharmaceutical compositions in the form of suppositories (e.g., with conventional suppository bases such as cocoa butter and other glycerides) or retention enemas for rectal delivery.

In one embodiment, the TRAIL receptor-binding agent is prepared with carriers that will protect the TRAIL receptor-binding agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers. These can be prepared according to methods known to those skilled in the art, e.g., as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of binding agent calculated to produce the desired therapeutic effect in association with the required pharmaceutical carrier. The specification for the dosage unit forms of the present technology are dictated by and directly dependent on the unique characteristics of the binding agent and the particular therapeutic effect to be achieved, and the limitations inherent in the art of compounding such TRAIL receptor-binding agent for the treatment of a subject.

The nucleic acid molecules of the present technology can be inserted into vectors and used as gene therapy vectors. Gene therapy vectors can be delivered to a subject by, e.g., intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

Measurement of TRAIL Receptor Binding.

In one embodiment, a TRAIL receptor binding assay refers to an assay format wherein a TRAIL receptor polypeptide and a TRAIL receptor-binding agent are mixed under conditions suitable for binding between the TRAIL receptor polypeptide and the TRAIL receptor-binding agent and assessing the amount of binding between the TRAIL receptor polypeptide and the TRAIL receptor-binding agent. The amount of binding is compared with a suitable control, which can be the amount of binding in the absence of the TRAIL receptor polypeptide, the amount of the binding in the presence of non-specific immunoglobulin composition, or both. The amount of binding can be assessed by any suitable method. Binding assay methods include, e.g., ELISA, radioreceptor binding assays, scintillation proximity assays, cell surface receptor binding assays, fluorescence energy transfer assays, liquid chromatography, membrane filtration assays, and the like. Biophysical assays for the direct measurement of TRAIL receptor polypeptide binding to TRAIL receptor-binding agents are, e.g., nuclear magnetic resonance, fluorescence, fluorescence polarization, surface plasmon resonance (BIACOR chips) and the like. Specific binding is determined by standard assays known in the art, e.g., radioligand binding assays, ELISA, FRET, immunoprecipitation, SPR, NMR (2D-NMR), mass spectroscopy and the like. If the specific binding of a candidate TRAIL receptor-binding agent is at least 1 percent greater than the binding observed in the absence of the candidate TRAIL receptor-binding agent, the candidate TRAIL receptor-binding agent is useful as a TRAIL receptor-binding agent.

Measurement of TRAIL Receptor-Binding Agent Biological Activity.

The TRAIL receptor-binding agents of the present technology, e.g., anti-TRAIL receptor antibodies and anti-TRAIL receptor antibody-related polypeptides, can be specified as agonists or antagonists for biological activities comprising specific activities disclosed herein. For example, TRAIL receptor agonists and antagonists, which are TRAIL receptor-binding agents can be made using methods known in the art. See e.g., WO 96/40281; U.S. Pat. No. 5,811,097; Deng et al., Blood 92: 1981-1988, 1998; Chen et al., Cancer Res., 58: 3668-3678, 1998; Harrop et al., J. Immunol. 161: 1786-1794, 1998; Zhu et al., Cancer Res., 58: 3209-3214, 1998; Yoon et al., J. Immunol., 160: 3170-3179, 1998; Prat et al., J. Cell. Sci., 111: 237-247, 1998; Pitard et al., J. Immunol. Methods, 205: 177-190, 1997; Liautard et al., Cytokinde, 9: 233-241, 1997; Carlson et al., J. Biol. Chem., 272: 11295-11301, 1997; Taryman et al., Neuron, 14: 755-762, 1995; Muller et al., Structure, 6: 1153-1167, 1998; Bartunek et al., Cytokinem, 8: 14-20, 1996. The biological activity, namely the agonist or antagonist properties of TRAIL receptor-binding agents can be characterized using any conventional in vivo and in vitro assays that have been developed to measure the biological activity of the TRAIL receptor polypeptide.

### VII. Uses of the Trail Receptor-Binding Agents

General.

The binding agents of the present technology are useful in methods known in the art relating to the localization and/or quantitation of a TRAIL receptor polypeptide (e.g., for use in measuring levels of the TRAIL receptor polypeptide within appropriate physiological samples, for use in diagnostic methods, for use in imaging the polypeptide, and the like). In one embodiment, TRAIL receptor-binding agents that contain the antibody derived binding domain, are useful as pharmacologically-active compositions (hereinafter “Therapeutics”). Binding agents are useful to isolate a TRAIL receptor polypeptide by standard techniques, such as affinity chromatography or immunoprecipitation. A TRAIL receptor-binding agent of the present technology can facilitate the purification of natural immunoreactive TRAIL receptor polypeptides or immunoreacitve TRAIL receptor-like polypeptides from biological samples, e.g., cells as well as recombinantly-produced immunoreactive TRAIL receptor polypeptides or TRAIL receptor-like polypeptides expressed in a host system. Moreover, TRAIL receptor-binding agent can be used to detect an immunoreactive TRAIL receptor polypeptide or an immunoreactive TRAIL receptor-like polypeptide (e.g., in a cellular lysate or cell supernatant) in order to evaluate the abundance and pattern of expression of the immunoreactive polypeptide. The TRAIL receptor-binding agents can be used diagnostically to monitor immunoreactive TRAIL receptor and/or immunoreactive TRAIL receptor-like immunoreactive polypeptide levels in tissue as part of a clinical testing procedure, e.g., to determine the efficacy of a given treatment regimen. As noted above, the detection can be facilitated by coupling (i.e., physically linking) the TRAIL receptor-binding agent to a detectable substance.

A. Detection and Quantitation of TRAIL Receptors

Detection of TRAIL Receptor Polypeptide Expression.

An exemplary method for detecting the presence or absence of a TRAIL receptor polypeptide or a TRAIL receptor-like polypeptide in a biological sample involves obtaining a biological sample from a test subject and contacting the biological sample with a TRAIL receptor-binding agent capable of detecting a TRAIL receptor polypeptide or a TRAIL receptor-like polypeptide such that the presence of a TRAIL receptor polypeptide or a TRAIL receptor-like polypeptide is detected in the biological sample. An example of a TRAIL receptor-binding agent is an antibody raised against SEQ ID NO: 15, capable of binding to a TRAIL receptor polypeptide or a TRAIL receptor-like polypeptide. In some embodiments, the TRAIL receptor binding agent is an antibody. In some embodiments, the antibody comprises a detectable label. The term “labeled”, with regard to the binding agent is intended to encompass direct labeling of the binding agent by coupling (i.e., physically linking) a detectable substance to the binding agent, as well as indirect labeling of the binding agent by reactivity with another compound that is directly labeled. Non-limiting examples of indirect labeling include detection of a primary antibody using a fluorescently-labeled secondary antibody,

In some embodiments, detection methods of the present technology can be used to detect a TRAIL receptor polypeptide or a TRAIL receptor-like polypeptide in a biological sample in vitro as well as in vivo. In vitro techniques for detection of a TRAIL receptor polypeptide or a TRAIL receptor-like polypeptide include enzyme linked immunosorbent assays (ELISAs), Western blots, immunoprecipitations, and immunofluorescence. Furthermore, in vivo techniques for detection of a TRAIL receptor polypeptide or a TRAIL receptor-like polypeptide include introducing into a subject a labeled TRAIL receptor-binding agent, e.g., an anti-TRAIL receptor antibody. For example, the antibody can be labeled with a radioactive marker whose presence and location in a subject can be detected by standard imaging techniques. In one embodiment, the biological sample contains polypeptide molecules from the test subject.

Immunoassay and Imaging.

A TRAIL receptor-binding agent of the present technology can be used to assay TRAIL receptor polypeptide levels or TRAIL receptor-like polypeptide levels in a biological sample using antibody-based techniques. For example, protein expression in tissues can be studied with classical immunohistological methods. Jalkanen, M. et al., J. Cell. Biol. 101: 976-985, 1985; Jalkanen, M. et al., J. Cell. Biol. 105: 3087-3096, 1987. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I), carbon (¹⁴C), sulfur (³⁵S), tritium (³H), indium (¹¹²I), and technetium (⁹⁹mTc), and fluorescent labels, such as fluorescein and rhodamine, and biotin.

In addition to assaying secreted TRAIL receptor polypeptide levels or TRAIL receptor-like polypeptide levels in a biological sample, secreted TRAIL receptor polypeptide levels or TRAIL receptor-like polypeptide levels can also be detected in vivo by imaging. A TRAIL receptor-binding agent, e.g., an anti-TRAIL receptor antibody labels or markers for in vivo imaging of the TRAIL receptor polypeptide levels or the TRAIL receptor-like polypeptide include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the TRAIL receptor-binding agent by labeling of nutrients for the relevant scFv clone.

A TRAIL receptor-binding agent which has been labeled with an appropriate detectable imaging moiety, such as a radioisotope (e.g., ¹³¹I, ¹¹²In, ^{99m}Tc), a radio-opaque substance, or a material detectable by nuclear magnetic resonance, is introduced (e.g., parenterally, subcutaneously, or intraperitoneally) into the subject. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled TRAIL receptor-binding agent will then preferentially accumulate at the location of cells which contain the specific target polypeptide. For example, in vivo tumor imaging is described in S. W. Burchiel et al., Tumor Imaging: The Radiochemical Detection of Cancer 13 (1982).

Thus, the present technology provides a diagnostic method of a medical condition, which involves: (a) assaying the expression of a polypeptide by measuring binding of a TRAIL receptor-binding agent of the present technology in cells or body fluid of an individual; (b) comparing the level of gene expression with a standard gene expression level, whereby an increase or decrease in the assayed polypeptide gene expression level compared to the standard expression level is indicative of a medical condition.

Diagnostic Uses.

The TRAIL receptor-binding compositions of the present technology are useful in diagnostic methods. As such, the present technology provides methods using the binding agents useful in the diagnosis of TRAIL receptor-related medical conditions in a subject. Binding agents may be selected such that they have any level of epitope binding specificity and very high binding affinity to the TRAIL receptor polypeptide. In general, the higher the binding affinity of an binding agent the more stringent wash conditions can be performed in an immunoassay to remove nonspecifically bound material without removing target polypeptide. Accordingly, TRAIL receptor-binding agents useful in diagnostic assays usually have binding affinities of at least 10⁸, 10⁹, 10¹⁰, 10¹¹ or 10¹² M⁻¹. Further, in some embodiments, it is desirable that TRAIL receptor-binding agents used as diagnostic reagents have a sufficient kinetic on-rate to reach equilibrium under standard conditions in at least 12 hours, preferably at least five (5) hours and more preferably at least one (1) hour.

Some methods of the present technology employ polyclonal preparations of anti-TRAIL receptor antibodies and anti-TRAIL receptor antibody compositions as diagnostic reagents, and other methods employ monoclonal isolates. The use of polyclonal mixtures has a number of advantages compared to compositions made of one monoclonal anti-TRAIL receptor antibody. By binding to multiple sites on a TRAIL receptor polypeptide, polyclonal anti-TRAIL receptor antibodies or other polypeptides, one can generate a stronger signal (for diagnostics) than a monoclonal that binds to a single site on the TRAIL receptor polypeptide or the TRAIL receptor-like polypeptide. Further, a polyclonal preparation can bind to numerous variants of a prototypical target sequence (e.g., allelic variants, species variants, strain variants, drug-induced escape variants) whereas a monoclonal antibody can bind only to the prototypical sequence or a narrower range of variants thereto. However, monoclonal anti-TRAIL receptor antibodies are advantageous for detecting a single antigen in the presence or potential presence of closely related antigens.

In methods employing polyclonal human anti-TRAIL receptor antibodies prepared in accordance with the methods described above, the preparation typically contains an assortment of TRAIL receptor-binding agents, e.g., antibodies, with different epitope specificities to the target polypeptide. In some methods employing monoclonal antibodies, it is desirable to have two antibodies of different epitope binding specificities. A difference in epitope binding specificities can be determined by a competition binding assay.

Although TRAIL receptor-binding agents which are human antibodies can be used as diagnostic reagents for any kind of sample, they are most useful as diagnostic reagents for human biological samples. TRAIL receptor-binding agents can be used to detect a given TRAIL receptor polypeptide in a variety of standard assay formats. Such formats include immunoprecipitation, Western blotting, ELISA, radioimmunoassay, and immunometric assays. See Harlow & Lane, Antibodies, A Laboratory Manual (Cold Spring Harbor Publications, New York, 1988); U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Biological samples can be obtained from any tissue or body fluid of a subject.

Immunometric or sandwich assays are a preferred format for the diagnostic methods of the present technology. See U.S. Pat. Nos. 4,376,110, 4,486,530, 5,914,241, and 5,965,375. Such assays use one TRAIL receptor-binding agent, e.g., an anti-TRAIL receptor antibody or a population of anti-TRAIL receptor antibodies immobilized to a solid phase, and another anti-TRAIL receptor antibody or a population of anti-TRAIL receptor antibodies. Typically, the solution anti-TRAIL receptor antibody or population of anti-TRAIL receptor antibodies is labeled. If an antibody population is used, the population typically contains antibodies binding to different epitope specificities within the target polypeptide. Accordingly, the same population can be used for both solid phase and solution antibody. If anti-TRAIL receptor monoclonal antibodies are used, first and second TRAIL receptor monoclonal antibodies having different binding specificities are used for the solid and solution phase. Solid phase and solution antibodies can be contacted with target antigen in either order or simultaneously. If the solid phase antibody is contacted first, the assay is referred to as being a forward assay. Conversely, if the solution antibody is contacted first, the assay is referred to as being a reverse assay. If the target is contacted with both antibodies simultaneously, the assay is referred to as a simultaneous assay. After contacting the TRAIL receptor polypeptide with the anti-TRAIL receptor antibody, a sample is incubated for a period that usually varies from about 10 min to about 24 hr and is usually about 1 hr. A wash step is then performed to remove components of the sample not specifically bound to the anti-TRAIL receptor antibody being used as a diagnostic reagent. When solid phase and solution antibodies are bound in separate steps, a wash can be performed after either or both binding steps. After washing, binding is quantified, typically by detecting label linked to the solid phase through binding of labeled solution antibody. Usually for a given pair of antibodies or populations of antibodies and given reaction conditions, a calibration curve is prepared from samples containing known concentrations of target antigen. Concentrations of the TRAIL receptor polypeptide in samples being tested are then read by interpolation from the calibration curve. Analyte can be measured either from the amount of labeled solution antibody bound at equilibrium or by kinetic measurements of bound labeled solution antibody at a series of time points before equilibrium is reached. The slope of such a curve is a measure of the concentration of the TRAIL receptor polypeptide in a sample

Suitable supports for use in the above methods include, e.g., nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™ (Amersham Pharmacia Biotech, Piscataway N.J., and the like Immobilization can be by absorption or by covalent attachment. Optionally, anti-TRAIL receptor antibodies can be joined to a linker molecule, such as biotin for attachment to a surface bound linker, such as avidin.

Predictive Medicine.

The present technology also pertains to the field of predictive medicine in which diagnostic assays, prognostic assays, pharmacogenomics, and monitoring clinical trials are used for prognostic (predictive) purposes to treat prophylactically a subject. Accordingly, one aspect of the present technology relates to diagnostic assays for determining TRAIL receptor polypeptide expression in a biological sample (e.g., blood, serum, cells, tissue) in order to determine whether subject is afflicted with a disease or disorder, or is at risk of developing a disorder, associated with aberrant TRAIL receptor polypeptide expression.

The present technology also provides for prognostic (or predictive) assays for determining whether an individual is at risk of developing a disorder associated with TRAIL receptor polypeptide expression or activity. Such assays can be used for prognostic or predictive purpose to thereby prophylactically treat an individual prior to the onset of a disorder characterized by or associated with a TRAIL receptor polypeptide. Furthermore, the methods of the present technology can also be used to assess whether an individual expresses a TRAIL receptor polypeptide or a polymorphic form of the TRAIL receptor polypeptide in instances where a TRAIL receptor-binding agent of the present technology has greater affinity for the TRAIL receptor polypeptide for its polymorphic form (or vice versa).

The levels of certain polypeptides in a particular tissue (or in the blood) of a subject may be indicative of the toxicity, efficacy, rate of clearance or rate of metabolism of a given drug when administered to the subject. The methods described herein can also be used to determine the levels of such polypeptide(s) in subjects to aid in predicting the response of such subjects to these drugs. Another aspect of the present technology provides methods for determining TRAIL receptor polypeptide expression in an individual to thereby select appropriate therapeutic or prophylactic compounds for that individual (referred to herein as “pharmacogenomics”). Pharmacogenomics allows for the selection of compounds (e.g., drugs) for therapeutic or prophylactic treatment of an individual based on the genotype of the individual (e.g., the genotype of the individual examined to determine the ability of the individual to respond to a particular compound).

The binding of a TRAIL receptor-binding agent of the present technology to a TRAIL receptor polypeptide or a TRAIL receptor-like polypeptide, e.g., can be utilized to identify a subject having or at risk of developing a disorder associated with the TRAIL receptor polypeptide or TRAIL receptor-like polypeptide expression or activity (which are described above). Alternatively, the prognostic assays can be utilized to identify a subject having or at risk for developing the disease or disorder. Thus, the present technology provides a method for identifying a disease or disorder associated with an aberrant TRAIL receptor polypeptide or TRAIL receptor-like polypeptide expression or activity in which a test sample is obtained from a subject and a TRAIL receptor-binding agent is detected, wherein the presence of an alteration of TRAIL receptor-binding agent is diagnostic for a subject having or at risk of developing a disease or disorder associated with an aberrant TRAIL receptor polypeptide or TRAIL receptor-like polypeptide expression or activity. As used herein, a “test sample” refers to a biological sample obtained from a subject of interest.

Furthermore, the prognostic assays described herein can be used to determine whether a subject can be administered a compound (e.g., an agonist, antagonist, peptidomimetic, polypeptide, peptide, nucleic acid, small molecule, or other drug candidate) to treat a disease or disorder associated with an aberrant TRAIL receptor polypeptide or TRAIL receptor-like polypeptide expression or activity. For example, such methods can be used to determine whether a subject can be effectively treated with a compound for a TRAIL receptor polypeptide or TRAIL receptor-like polypeptide-associated disorder. Thus, the present technology provides methods for determining whether a subject can be effectively treated with a compound for a disorder associated with an aberrant TRAIL receptor polypeptide or TRAIL receptor-like polypeptide expression or activity in which a test sample is obtained and the TRAIL receptor polypeptide or the TRAIL receptor-like polypeptide is detected using the TRAIL receptor-binding agent (e.g., wherein the presence of the TRAIL receptor polypeptide or the TRAIL receptor-like polypeptide is diagnostic for a subject that can be administered the compound to treat a disorder associated with an aberrant TRAIL receptor polypeptide or TRAIL receptor-like polypeptide expression or activity).

The level of the TRAIL receptor polypeptide or the TRAIL receptor-like polypeptide in a blood or tissue sample obtained from a subject is determined and compared with the level found in a blood sample or a sample from the same tissue type obtained from an individual who is free of the disease. An overabundance (or underabundance) of the TRAIL receptor polypeptide or TRAIL receptor-like polypeptide in the sample obtained from the subject suspected of having the TRAIL receptor polypeptide or TRAIL receptor-like polypeptide-associated disease compared with the sample obtained from the healthy subject is indicative of the TRAIL receptor polypeptide or TRAIL receptor-like polypeptide-associated disease in the subject being tested. Further testing may be required to make a positive diagnosis.

There are a number of diseases in which the degree of overexpression (or underexpression) of certain TRAIL receptor polypeptide or TRAIL receptor-like polypeptide molecules known to be indicative of whether a subject with the disease is likely to respond to a particular type of therapy or treatment. Thus, the method of detecting a TRAIL receptor polypeptide or TRAIL receptor-like polypeptide in a sample can be used as a method of prognosis, e.g., to evaluate the likelihood that the subject will respond to the therapy or treatment. The level of the relevant prognostic polypeptide in a suitable tissue or blood sample from the subject is determined and compared with a suitable control, e.g., the level in subjects with the same disease but who have responded favorably to the treatment. The degree to which the prognostic polypeptide is overexpressed (or underexpressed) in the sample compared with the control may be predictive of likelihood that the subject will not respond favorably to the treatment or therapy. The greater the overexpression (or underexpression) relative to the control, the less likely the subject will respond to the treatment.

There are a number of diseases in which the degree of overexpression (or underexpression) of certain target polypeptides, referred to herein as “predictive polypeptides,” is known to be indicative of whether a subject will develop a disease. Thus, the method of detecting a TRAIL receptor polypeptide or TRAIL receptor-like polypeptide in a sample can be used as a method of predicting whether a subject will develop a disease. The level of the relevant predictive polypeptide in a suitable tissue or blood sample from a subject at risk of developing the disease is determined and compared with a suitable control, e.g., the level in subjects who are not at risk of developing the disease. The degree to which the predictive polypeptide is overexpressed (or underexpressed) in the sample compared with the control may be predictive of likelihood that the subject will develop the disease. The greater the overexpression (or underexpression) relative to the control, the more likely the subject will development the disease.

The methods described herein can be performed, e.g., by utilizing pre-packaged diagnostic kits comprising at least one probe reagent, e.g., TRAIL receptor-binding agent described herein, which can be conveniently used, e.g., in clinical settings to diagnose subjects exhibiting symptoms or family history of a disease or illness involving a TRAIL receptor polypeptide or TRAIL receptor-like polypeptide. Furthermore, any cell type or tissue in which TRAIL receptor polypeptide or TRAIL receptor-like polypeptide is expressed can be utilized in the prognostic assays described herein.

B. Prophylactic and Therapeutic Uses of TRAIL receptor-Binding Agents

1. General

The TRAIL receptor-binding agents of the present present technology (e.g., monoclonal antibodies having the same epitope specificity as the mouse-mouse hybridoma CTB006 having CGMCC Accession No. 1691; monoclonal antibodies having a heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; a monoclonal antibody having the heavy chain amino acid sequence set forth in SEQ ID NO: 14 and the light chain amino acid sequence set forth in SEQ ID NO: 12; the HuCTB006 antibody) are useful to prevent or treat disease. In some embodiments, the present technology provides for prophylactic and/or therapeutic methods of treating a subject at risk of (or susceptible to) a disorder or having a disorder associated with an aberrant TRAIL receptor-binding agent expression or activity. Accordingly, the present technology provides methods for the prevention and/or treatment of a TRAIL receptor-related medical condition in a subject comprising administering an effective amount of a TRAIL receptor-binding agent to a subject in need thereof. For example, a subject can be administered a TRAIL receptor-binding agent compositions of the present technology in an effort to replace absent or decreased levels of the TRAIL receptor polypeptide (e.g., insulin), to supplement absent or decreased levels of a different polypeptide (e.g., an anti-TRAIL receptor antibody), to inhibit the activity of a polypeptide (e.g., an oncogene), to activate the activity of a TRAIL receptor polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

The TRAIL receptor-binding agents of the present technology are useful in prophylactic and therapeutic applications in a variety of disorders in a subject including, but not limited to: those treating, inhibiting or preventing diseases, disorders or conditions including malignant diseases, disorders, or conditions associated with such diseases or disorder such as diseases associated with increased cell survival, or the inhibition of apoptosis, for example cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune disorders (such as, multiple sclerosis, Sjogren's syndrome, Grave's disease, Hashimoto's thyroiditis, autoimmune diabetes, biliary cirrhosis, Behcet's disease, Crohn's disease, polymyositis, systemic lupus erythematosus and immune-related glomerulonephritis, autoimmune gastritis, autoimmune thrombocytopenic purpura, and rheumatoid arthritis) and viral infections (such as herpes viruses, pox viruses and adenoviruses), inflammation, graft vs. host disease (acute and/or chronic), acute graft rejection, and chronic graft rejection. Antigen binding polypeptides, variants or derivatives thereof of the present disclosure are used to inhibit growth, progression, and/or metastasis of cancers, in particular those listed above or in the paragraph that follows.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

The TRAIL receptor-binding agents of the present technology are useful in potential prophylactic and therapeutic applications in a subject including, but not limited to: those involving development, differentiation, and activation of bone cells; in diseases or pathologies of cells in blood circulation such as red blood cells and platelets; various immunological disorders and/or pathologies; pulmonary diseases and disorders; autoimmune and inflammatory diseases; cardiovascular diseases; metabolic diseases; reproductive diseases, renal diseases, diabetes, brain trauma, cancer growth and metastasis; viral infections, cancer therapy, periodontal disease; tissue regeneration; acute lymphoblastic leukemia; gliomas; neurologic diseases; neurodegenerative disorders; Alzheimer's disease; Parkinson's disorder; and hematopoietic disorders.

In some embodiments, a pharmaceutically effective amount of an anti-TRAIL-R2 antibody induces cell death by contact with a target cell. A pharmaceutically effective amount of an antibody recognizing TRAIL-R2 or a humanized antibody recognizing TRAIL-R2 is an amount administered to an individual sufficient to cause a desired effect. Desired effects of administration of a pharmaceutically effective amount of TRAIL-R2 recognizing antibodies include death of a target cell, growth inhibition of a target cell, stimulation of TRAIL-R2, and binding to TRAIL-R2 in a target cell. A target cell is a cell that expresses TRAIL-R2 and includes, by way of example, abnormally growing cells such as human carcinoma cells and leukemia cells. Also included is a cell with a pathological condition, in which those where cell proliferation is abnormal or dysregulated such as malignant or benign cancer. Accordingly, in some embodiments, the anti-TRAIL receptor binding agents are useful in methods for the prevention or treatment of the growth and/or metastisis of cancers, e.g., but not limited to, breast cancer, liver cancer, prostate cancer, ovarian cancer, lung cancer, brain cancer, pancreatic cancer, and colorectal cancer, in subjects in need thereof. In one embodiment, the TRAIL receptor-binding agents have in vitro apoptosis-inducing activity wherein the binding agent can induce at least 30% cell death at the concentrations equal or lower than 10 μg/ml, preferably at least 50%, 70%, 90%, more preferably 100% cell death. In one embodiment, the TRAIL receptor-binding agents have in vivo apoptosis-inducing activity wherein the binding agent can reduce at least 30% tumor size in human cancer xenograft models when treated with the doses equal or less than 10 mg/kg body weight, preferably, at least 50%, 70%, 90%, more preferably 100%.

When used in vivo for therapy, the TRAIL receptor-binding agents, e.g., the anti-TRAIL receptor antibodies of the present technology are administered to the subject in effective amounts (i.e., amounts that have desired therapeutic effect). In some embodiments, administration is parenterally. For parenteral administration, in some embodiments, the TRAIL receptor-binding agent will be formulated in a unit dosage injectable form (solution, suspension, emulsion) in association with a pharmaceutically-acceptable parenteral vehicle. Such vehicles are inherently nontoxic, and non-therapeutic.

The dose and dosage regimen will depend upon the degree of the TRAIL receptor-related disease or disorder, the characteristics of the particular TRAIL receptor-binding agent used, e.g., its therapeutic index, the subject, and the subject's history. In some embodiments, the TRAIL receptor-binding agent is administered continuously over a period of 1-2 weeks, intravenously to treat cells in the vasculature and subcutaneously and intraperitoneally to treat regional lymph nodes.

Use of anti-TRAIL receptor IgM antibodies can be preferred for certain applications. However, IgG molecules by being smaller can be more able than IgM molecules to localize to certain types of infected cells. There is evidence that complement activation in vivo leads to a variety of biological effects, including the induction of an inflammatory response and the activation of macrophages (Unanue and Benecerraf, Textbook of Immunology, 2nd Edition, Williams & Wilkins, p. 218 (1984)). The increased vasodilation accompanying inflammation can increase the ability of various agents to localize in infected cells. Therefore, TRAIL receptor-antibody combinations of the type specified by the present technology can be used therapeutically in many ways. Additionally, antigen, e.g., purified TRAIL receptor polypeptide, fragments or analogs thereof, (Hakomori, Ann. Rev. Immunol. 2: 103, 1984) or anti-idiotypic antibodies (Nepom et al., Proc. Natl. Acad. Sci. USA 81: 2864, 1985; Koprowski et al., Proc. Natl. Acad. Sci. USA 81: 216, 1984) relating to such antigens could be used to induce an active immune response in human subjects. Such a response includes the formation of antibodies capable of activating human complement for a desirable biological effect, e.g., target cell destruction.

2. Diseases and Disorders Characterized by Increased Levels of TRAIL Receptor

Diseases and disorders that are characterized by increased (relative to a subject not suffering from the disease or disorder) levels or biological activity of a TRAIL receptor polypeptide can be treated with a TRAIL receptor-binding agent-based therapeutic compounds that antagonize (i.e., reduce or inhibit) activity, which can be administered in a therapeutic or prophylactic manner Therapeutic compounds that can be utilized include, but are not limited to: (i) an aforementioned TRAIL receptor-binding agent; and (ii) nucleic acids encoding a TRAIL receptor-binding agent.

Diseases and disorders that are characterized by decreased (relative to a subject not suffering from the disease or disorder) levels or biological activity of a TRAIL receptor polypeptide can be treated with a TRAIL receptor-binding agent-based therapeutic compounds that increase (i.e., are agonists to) the TRAIL receptor activity. Therapeutics that upregulate activity can be administered in a therapeutic or prophylactic manner Therapeutics that can be utilized include, but are not limited to, a TRAIL receptor-binding agent that increases bioavailability.

Increased or decreased levels can be readily detected by quantifying the TRAIL receptor-binding agent-induced peptides and/or RNA, by obtaining a subject's tissue sample (e.g., from biopsy tissue) and assaying it in vitro for RNA or peptide levels, structure and/or activity of the expressed TRAIL receptor polypeptide (or mRNAs of an aforementioned polypeptide). Methods that are well known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

3. Prophylactic Methods

In one aspect, the present technology provides a method for preventing, in a subject, a disease or condition associated with an aberrant TRAIL receptor expression or activity, by administering to the subject a TRAIL receptor-binding agent (e.g., monoclonal antibodies having the same epitope specificity as the mouse-mouse hybridoma CTB006 having CGMCC Accession No. 1691; monoclonal antibodies having a heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; a monoclonal antibody having the heavy chain amino acid sequence set forth in SEQ ID NO: 14 and the light chain amino acid sequence set forth in SEQ ID NO: 12; the HuCTB006 antibody) that modulates TRAIL receptor polypeptide expression or at least one TRAIL receptor polypeptide activity.

Subjects at risk for a disease that is caused or contributed to by aberrant TRAIL receptor polypeptide expression or activity can be identified by, e.g., any or a combination of diagnostic or prognostic assays as described herein. In prophylactic applications, pharmaceutical compositions or medicaments of TRAIL receptor-binding agents are administered to a subject susceptible to, or otherwise at risk of a disease or condition (i.e., an immune disease) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. Administration of a prophylactic TRAIL receptor-binding agent (e.g., monoclonal antibodies having the same epitope specificity as the mouse-mouse hybridoma CTB006 having CGMCC Accession No. 1691; monoclonal antibodies having a heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; a monoclonal antibody having the heavy chain amino acid sequence set forth in SEQ ID NO: 14 and the light chain amino acid sequence set forth in SEQ ID NO: 12; the HuCTB006 antibody) can occur prior to the manifestation of symptoms characteristic of the aberrancy, such that a disease or disorder is prevented or, alternatively, delayed in its progression. Depending upon the type of aberrancy, e.g., a TRAIL receptor-binding agent which acts as a TRAIL receptor agonist or a TRAIL receptor antagonist can be used for treating the subject. The appropriate compound can be determined based on screening assays described herein.

4. Therapeutic Methods

Another aspect of the present technology includes methods of modulating TRAIL receptor polypeptide expression or activity in a subject for therapeutic purposes. The modulatory method of the present technology involves contacting a cell with a TRAIL receptor-binding agent (e.g., monoclonal antibodies having the same epitope specificity as the mouse-mouse hybridoma CTB006 having CGMCC Accession No. 1691; monoclonal antibodies having a heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; a monoclonal antibody having the heavy chain amino acid sequence set forth in SEQ ID NO: 14 and the light chain amino acid sequence set forth in SEQ ID NO: 12; the HuCTB006 antibody), that modulates one or more of the activities of the TRAIL receptor polypeptide activity associated with the cell. In therapeutic applications, compositions or medicants are administered to a subject suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose.

A compound that modulates a TRAIL receptor polypeptide activity is described herein, and may include, e.g., a nucleic acid encoding a TRAIL receptor-binding agent or a TRAIL receptor-binding agent-related polypeptide. In one embodiment, the TRAIL receptor-binding agent stimulates one or more TRAIL receptor polypeptide activity. Examples of such stimulatory compounds include a TRAIL receptor-binding agent and a nucleic acid molecule encoding a TRAIL receptor-binding agent that has been introduced into the cell. In another embodiment, the TRAIL receptor-binding agent inhibits one or more TRAIL receptor polypeptide activity. These modulatory methods can be performed in vitro (e.g., by culturing the cell with the TRAIL receptor-binding agent) or, alternatively, in vivo (e.g., by administering the TRAIL receptor-binding agent to a subject). As such, the present technology provides methods of treating an individual afflicted with a TRAIL receptor-associated disease or disorder characterized by aberrant expression or activity of a TRAIL receptor polyepeptide or nucleic acid molecules encoding a TRAIL receptor polypeptide. In one embodiment, the method involves administering a TRAIL receptor-binding agent (e.g., a compound identified by a screening assay described herein), or combination TRAIL receptor-binding agents that modulates (e.g., up-regulates or down-regulates) TRAIL receptor polypeptide expression or activity. In another embodiment, the method involves administering a TRAIL receptor-binding agent or a nucleic acid molecule encoding a TRAIL receptor-binding agent as therapy to compensate for reduced or aberrant TRAIL receptor polypeptide expression or activity. Stimulation of TRAIL receptor polypeptide activity is desirable in situations in which TRAIL receptor polypeptide is abnormally downregulated.

5. Cancer and Malignancy Therapy

As noted above, the TRAIL receptor binding agents of the present technology (e.g., monoclonal antibodies having the same epitope specificity as the mouse-mouse hybridoma CTB006 having CGMCC Accession No. 1691; monoclonal antibodies having a heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; a monoclonal antibody having the heavy chain amino acid sequence set forth in SEQ ID NO: 14 and the light chain amino acid sequence set forth in SEQ ID NO: 12; the HuCTB006 antibody), are useful in the context of therapeutic and prophylactic treatment methods. A mouse-mouse hybridoma, CTB006, that produces antibodies of the present technology has been deposited and an Accession Number CGMCC1691 has been assigned.

The majority of tumor cells, e.g., such as those detailed herein, express cell surface TRAIL-R2 and are susceptible to the TRAIL receptor binding agents of the present technology. In the context of a malignancy therapy, the antibodies of the present technology, (e.g., monoclonal antibodies having the same epitope specificity as the mouse-mouse hybridoma CTB006 having CGMCC Accession No. 1691; monoclonal antibodies having a heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; a monoclonal antibody having the heavy chain amino acid sequence set forth in SEQ ID NO: 14 and the light chain amino acid sequence set forth in SEQ ID NO: 12; the HuCTB006 antibody), are able to induce apoptosis of TRAIL-R2 expressing tumor cells, and exhibit strong tumoricidal activity in vivo. As such, the TRAIL receptor binding agents of the present technology are useful for the treatment of cancer in subjects in need thereof.

With respect to cancer therapy, the TRAIL receptor binding agents of the present technology display surprising and unexpected synergy when administered with a chemotherapeutic agent. That is, the combination of TRAIL receptor binding agent and chemotherapeutic agent results in a greater effect than with either compound alone, or what would be an additive effect of the combination. By way of example, but not by way of limitation, the synergistic cytotoxic effect of the combination results in one or more of greater tumor regression, less tumor growth, reduced tumor volume, greater tumor growth inhibition, and improved subject health (e.g., less loss of body weight) than the administration of a chemotherapeutic agent alone or the TRAIL receptor binding agents alone. Additionally or alternatively, in some embodiments, the combination allows for a lower dose of either the TRAIL receptor binding agent, the chemotherapeutic agent or both to be administered, to achieve the same or better result than with the individual agents alone. Additionally or alternatively, in some embodiments, treatment with a combination of a TRAIL receptor binding agent, e.g., monoclonal antibodies having the same epitope specificity as the mouse-mouse hybridoma CTB006 having CGMCC Accession No. 1691 (e.g., HuCTB006) and a chemotherapeutic agent reduces or eliminates a tumor or tumor growth more effectively (e.g., in less time, with lower dose, with fewer doses or with fewer treatments) or prevents or reduces metastasis more effectively than treatment with either compound alone.

As noted above, the TRAIL receptor-binding agents disclosed herein provide surprising and unexpected synergistic effect, or even significant synergistic effect, when used in combination with chemotherapeutic agents for the treatment of cancer, for the killing of tumor cells and/or for inhibiting the proliferation of tumor cells. By way of example, but not by way of limitation, in some embodiments, the combination therapy of TRAIL receptor-binding agents and chemotherapeutic agents has significantly synergistic or synergistic cytotoxic effect on the viability of tumor cells.

Additionally or alternatively, in some embodiments, the synergistic effect the combination therapy of TRAIL receptor-binding agents and chemotherapeutic agents is with respect to tumor volume and tumor weight.

In some embodiments, the cancer/tumor is one or more of colon cancer, lung cancer, liver cancer, pancreatic cancer, breast cancer, ovarian cancer, stomach cancer, and leukaemia.

In some embodiments the chemotherapeutic agent that is used in combination with the TRAIL receptor binding agents is one or more of vinca alkaloids, agents that disrupt microtubule formation (such as colchicines and its derivatives), anti-angiogenic agents, therapeutic antibodies, EGFR targeting agents, tyrosine kinase targeting agent (such as tyrosine kinase inhibitors), transitional metal complexes, proteasome inhibitors, antimetabolites (such as nucleoside analogs), alkylating agents, platinum-based agents, anthracycline antibiotics, topoisomerase inhibitors, macrolides, therapeutic antibodies, retinoids (such as all-trans retinoic acids or a derivatives thereof); geldanamycin or a derivative thereof (such as 17-AAG), adriamycin, colchicine, cyclophosphamide, actinomycin, bleomycin, duanorubicin, doxorubicin, epirubicin, mitomycin, methotrexate, mitoxantrone, fluorouracil, carboplatin, carmustine (BCNU), methyl-CCNU, cisplatin, etoposide, interferons, camptothecin and derivatives thereof, phenesterine, taxanes and derivatives thereof (e.g., taxol, paclitaxel and derivatives thereof, taxotere and derivatives thereof, and the like), topetecan, vinblastine, vincristine, tamoxifen, piposulfan, nab-5404, nab-5800, nab-5801, Irinotecan, HKP, Ortataxel, gemcitabine, Oxaliplatin, Herceptin®, vinorelbine, Doxil®, capecitabine, Alimta®, Avastin®, Velcade®, Tarceva®, Neulasta®, lapatinib, sorafenib, erlotinib, erbitux, nanoparticles comprising a thiocolchicine derivative and a carrier protein (such as albumin), an antineoplastic agent including, but is not limited to, carboplatin, Navelbine® (vinorelbine), anthracycline (Doxil®), lapatinib (GW57016), Herceptin®, gemcitabine (Gemzar®), capecitabine (Xeloda®), Alimta®, cisplatin, 5-fluorouracil (5-Fu), epirubicin, cyclophosphamide, derivatives thereof, and other standard chemotherapeutic agents well recognized in the art. In some embodiments, the chemotherapeutic agent is 5-fluorouracil or taxol.

Given the surprising and unexpected synergistic effects, in some embodiments, the combination therapy allows for a lower dose or fewer doses of a chemotherapeutic agent, thereby decreasing toxicity to normal cells. In this regard, in some embodiments, the administration of a TRAIL receptor binding agent of the present technology is made during the course of chemotherapy (such as combined cycles of radiation, chemotherapeutic treatment) along with the administration of tumor necrosis factor, interferon or other cytoprotective or immunomodulatory agent. As such, the TRAIL receptor binding agents of the present technology and a compound useful in adjunct therapy may be administrated simultaneously and sequentially or separately to a subject in need of administration thereof.

6. Determination of the Biological Effect of the TRAIL Receptor-Binding Agent-Based Therapeutic

In various embodiments of the present technology, suitable in vitro or in vivo assays are performed to determine the effect of a specific TRAIL receptor-binding agent-based therapeutic and whether its administration is indicated for treatment of the affected tissue in a subject.

In various embodiments, in vitro assays can be performed with representative cells of the type(s) involved in the subject's disorder, to determine if a given TRAIL receptor-binding agent-based therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

7. Treatment Regime and Effective Dosages

Some compositions include a combination of multiple (e.g., two or more) TRAIL receptor-binding agents of the present technology. In some compositions, each of the TRAIL receptor-binding agents thereof of the composition is a monoclonal antibody or a human sequence antibody that binds to a distinct, pre-selected epitope of a TRAIL receptor polypeptide.

Effective doses of the TRAIL receptor-binding agents of the present technology, e.g., anti-TRAIL receptor antibodies or anti-TRAIL receptor antibody cytotoxin conjugates, for the treatment of TRAIL receptor-related conditions and diseases described herein vary depending upon many different factors, including means of administration, target site, physiological state of the subject, whether the subject is human or an animal, other medications administered, and whether treatment is prophylactic or therapeutic. Usually, the subject is a human but nonhuman mammals including transgenic mammals can also be treated. Treatment dosages need to be titrated to optimize safety and efficacy.

Typically, an effective amount of the compositions of the present technology, sufficient for achieving a therapeutic or prophylactic effect, range from about 0.000001 mg per kilogram body weight per day to about 10,000 mg per kilogram body weight per day. In some embodiments, the dosage ranges are from about 0.0001 mg per kilogram body weight per day to about 100 mg per kilogram body weight per day. For administration with a TRAIL receptor-binding agent, e.g., an anti-TRAIL receptor antibody, the dosage ranges from about 0.0001 to 100 mg/kg per week, or about 0.01 to 5 mg/kg per week of the host body weight. For example dosages can be 1 mg/kg body weight or 10 mg/kg body weight per week or within the range of 1-10 mg/kg per week. In some embodiments, a single dosage of antibody ranges from 0.1-10,000 micrograms per kg body weight. In some embodiments, antibody concentrations in a carrier ranges from 0.2 to 2000 micrograms per delivered milliliter. An exemplary treatment regime entails administration once per every two weeks or once a month or once every 3 to 6 months. In some methods, two or more TRAIL receptor binding agents with different binding specificities are administered simultaneously, in which case the dosage of each antibody administered falls within the ranges indicated. A TRAIL receptor-binding agent, e.g., an anti-TRAIL receptor antibody is usually administered on multiple occasions. Intervals between single dosages can be weekly, monthly or yearly. Intervals can also be irregular as indicated by measuring blood levels of antibody in the subject. In some methods, dosage is adjusted to achieve a plasma TRAIL receptor-binding agent, e.g., an anti-TRAIL receptor antibody concentration, of 1-1000 μg/ml and in some methods 25-300 μg/ml. Alternatively, a TRAIL receptor-binding agent, e.g., an anti-TRAIL receptor antibody, can be administered as a sustained release formulation, in which case less frequent administration is required. Dosage and frequency vary depending on the half-life of the TRAIL receptor-binding agent in the subject. In general, human anti-TRAIL receptor antibodies show the longest half life, followed by humanized anti-TRAIL receptor antibodies, chimeric anti-TRAIL receptor antibodies, and nonhuman anti-TRAIL receptor antibodies. The dosage and frequency of administration can vary depending on whether the treatment is prophylactic or therapeutic. In prophylactic applications, a relatively low dosage is administered at relatively infrequent intervals over a long period of time. Some subjects continue to receive treatment for the rest of their lives. In therapeutic applications, a relatively high dosage at relatively short intervals is sometimes required until progression of the disease is reduced or terminated, and preferably until the subject shows partial or complete amelioration of symptoms of disease. Thereafter, the patent can be administered a prophylactic regime. Doses for nucleic acids encoding TRAIL receptor immunogens range from about 10 ng to 1 g, 100 ng to 100 mg, 1 μg to 10 mg, or 30-300 μg DNA per subject. Doses for infectious viral vectors vary from 10-100, or more, virions per dose.

8. Toxicity

In some embodiments, an effective amount (e.g., dose) of the TRAIL receptor-binding agents described herein will provide therapeutic benefit without causing substantial toxicity to the subject. Toxicity of the TRAIL receptor-binding agent described herein can be determined by standard pharmaceutical procedures in cell cultures or experimental animals, e.g by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the TRAIL receptor-binding agent described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the subject's condition. See, e.g., Fingl et al., In: The Pharmacological Basis of Therapeutics, Ch. 1 (1975).

9. Kits

Also within the scope of the present technology are kits comprising the TRAIL receptor-binding agent compositions (e.g., monoclonal antibodies having the same epitope specificity as the mouse-mouse hybridoma CTB006 having CGMCC Accession No. 1691; monoclonal antibodies having a heavy chain CDR amino acid sequences SYFIH as set forth in SEQ ID NO: 8, WIYPGNVNTKYSEKFKG as set forth in SEQ ID NO: 9, and GEAGYFD as set forth in SEQ ID NO: 10, and light chain CDR amino acid sequences KASQDVSTAVA as set forth in SEQ ID NO: 3, WASTRHT as set forth in SEQ ID NO: 4, and QQHYRTPW as set forth in SEQ ID NO: 5; a monoclonal antibody having the heavy chain amino acid sequence set forth in SEQ ID NO: 14 and the light chain amino acid sequence set forth in SEQ ID NO: 12; the HuCTB006 antibody) and instructions for use. The kits are useful for detecting the presence of a TRAIL receptor polypeptide or a TRAIL receptor-like polypeptide in a biological sample. For example, the kit can comprise: a labeled TRAIL receptor-binding agent capable of binding a TRAIL receptor polypeptide or a TRAIL receptor-like polypeptide in a biological sample; means for determining the amount of the TRAIL receptor polypeptide or TRAIL receptor-like polypeptide in the sample; and means for comparing the amount of the TRAIL receptor polypeptide or the TRAIL receptor-like polypeptide in the sample with a standard. The kit components, (e.g., reagents) can be packaged in a suitable container. The kit can further comprise instructions for using the kit to detect the TRAIL receptor polypeptide or the TRAIL receptor-like polypeptide.

In one embodiment, a kit of the present technology comprises the combination of the TRAIL receptor binding agent of the present technology with a disease inhibiting compound, wherein the compound is anti-tumor drug such radioisotope, chemotherapeutic agent, therapeutic antibody or cytokine. In some embodiments the disease inhibiting compound is a chemotherapeutic agent. In some embodiments, the chemotherapeutic agent is taxol or 5-fluorouracil.

The following EXAMPLES are presented in order to more fully illustrate the preferred embodiments of the present technology. These EXAMPLEs should in no way be construed as limiting the scope of the present technology, as defined by the appended claims.

### EXAMPLES

### Example 1. Characterization of the Binding Specificity and Affinity of Hu CTB006

1.1 Binding Specificity

The binding specificity of HuCTB006 was evaluated by chemiluminescent enzyme immunoassay (CLEIA). DR4-His-EC (Lot: 090222-P, 0.72 mg/mL, E coli.), DcR1-His (recombinant human TRAIL R3/TNFRSF10C, Novoprotein Science, Lot: 0328670), DcR2-His (recombinant human TRAIL R4/TNFRSF10D, Sino Biological, Lot: LC06JU1502), and OPG-His (recombinant human osteroprotegerin/TNFRSF11B, Wuxi Pharmatech Co. Ltd, Lot: 20120717) were coated on the microplate, which was then blocked.

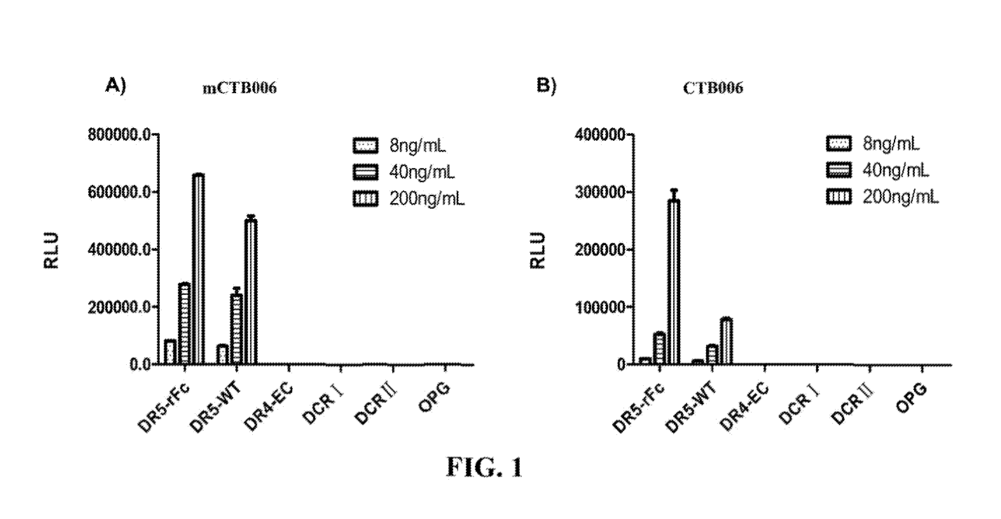

HuCTB006 was diluted into 200, 40 and 8 ng/mL and then added to the wells. After 1 hour incubation in 37° C., the plate was washed. The binding of HuCTB006 with immobilized antigens was detected with HRP labeled secondary antibody and a substrate solution. The results showed no cross-reaction of mCTB006 or HuCTB006 with those coated-antigens as shown in FIG. 1, which indicated that HuCTB006 had a good binding specificity.

1.2 Affinity

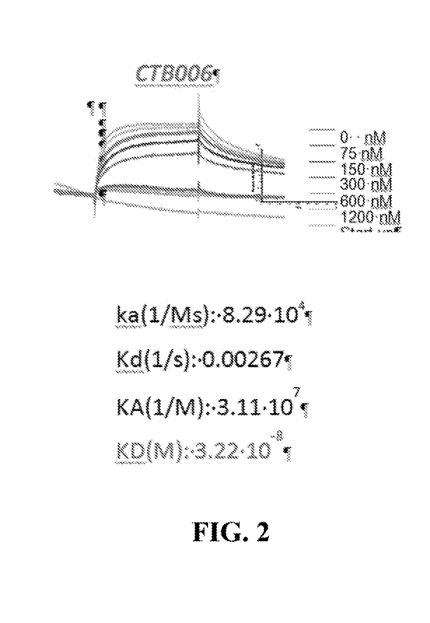

The binding affinity of HuCTB006 to DR5 was determined by surface plasmon resonance (SPR). In SPR assay, HuCTB006 was immobilized to a CMS BIAcore sensorchip using standard amine coupling. Non-cross-linked proteins were removed, and unreacted sites were blocked with ethanolamine. A series concentration of purified recombinant human DR5-rFc protein flowed over the sensor surface continuously. Between the runs, the sensor surface was regenerated with 10 mM Glycine-HCl (pH 2.5). The bindings of hDR5-rFc to the activated surface were detected through the changes in the index of refraction at the surface and recorded as RU (i.e., resonance units). Curves were generated from the RU trace and evaluated by fitting algorithms, which compared the raw data to well-defined binding models (BIAevalution™ 2.0 software) as shown in FIG. 2.

### Example 2. In Vitro Efficacy of HuCTB006 Treatments

2.1 Cytotoxicity of HuCTB006 on Human Normal Tissue Cell Lines

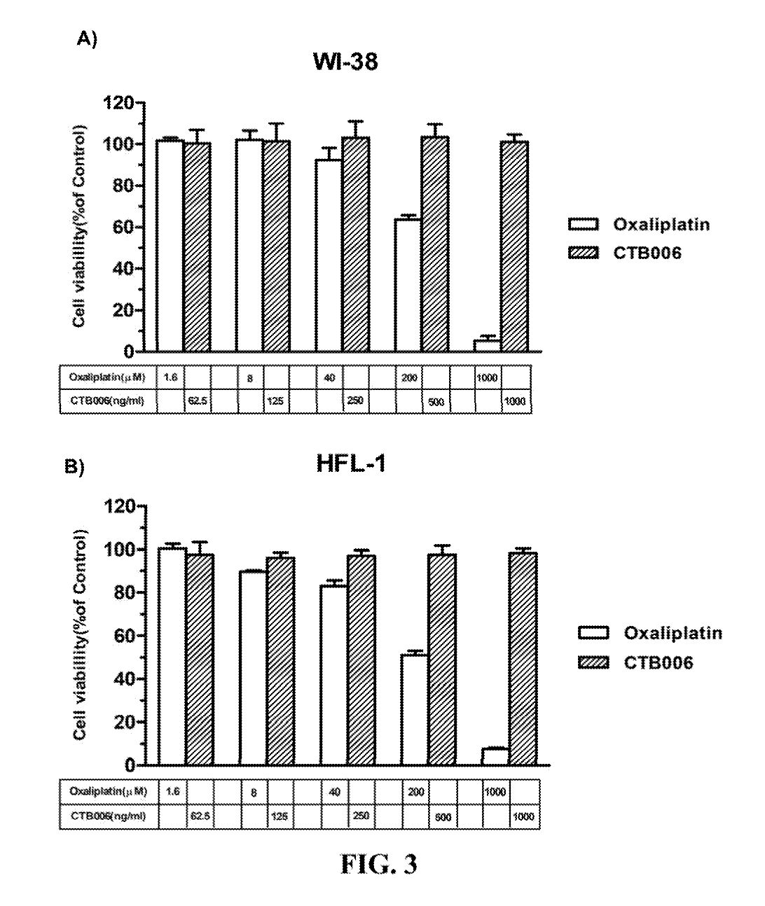

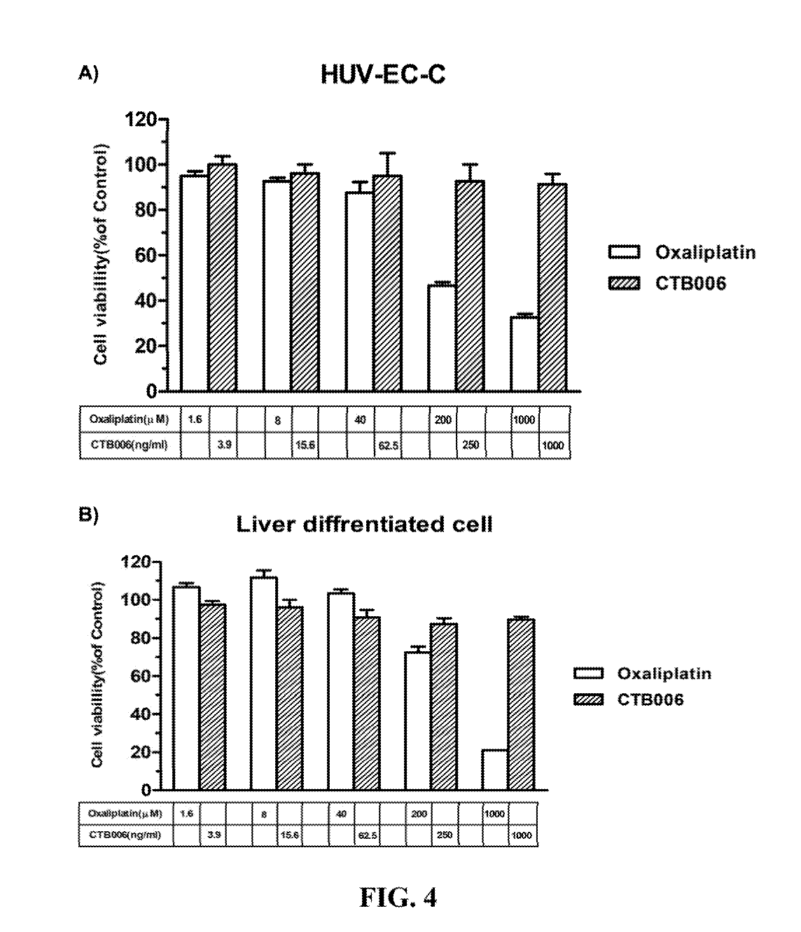

Several human normal tissue cell lines, including human lung epithelial fibroblasts WI-38, human embryonic lung fibroblasts HFL-1, human umbilical vein epithelial cells HUV-EC-C, and human hepatic differentiated cells, which were derived from liver genitor cells (obtained from Peking University Third Hospital). The cells were cultured at 37° C. and 5% of CO₂, and in culture media containing 10% of FBS. The culture medium for WI-38 was MEM, and the culture medium for HFL-1 and HUV-EC-C was F-12K. The cells were treated with oxaliplatin for 24 hours, then different concentrations of CTB006 were added to the combined group. The results were determined at 24 hours after the treatments, shown in FIG. 3 & FIG. 4. The human normal tissue cells examined were insensitive to CTB006, indicating there was no cytotoxicity of HuCTB006 on human normal tissue cells.

2.2 Cytotoxicity of CTB006 on Human Tumor Cell Lines

Eighteen human tumor cell lines were treated with 62.5, 125, 250, 500, or 1000 ng/mL HuCTB006 or different concentrations of chemotherapy drugs, which act as positive controls. The cells were cultured at 37° C. and 5% of CO₂, and in culture media containing 10% of FBS. The culture medium for SK-Hep-1, HepG2, HCT116, SW480, WiDr (1×NEAA), MIA-PaCa-2 (2.5% Horse serum) MDA-MB-231 (1 mM NaPyr, 1×MEM-NEAA, 1×MEM VITAMIN) was DMEM. The culture medium for COLO205, BXPC3 (2 mM glutamin, 10 mM HEPES), Panc2.03 (15FBS, 1 mM NaPyr, 1×MEM-NEAA, 10 mM human insulin, 10 mM HEPES), H2122, OVCAR3 (1 mM NaPyr, 10 ug/ml human insulin, 10 mM HEPES, 2.5 g/L glucose), Molt-4 (HEPES, 2.5 g/l glocose), Jurkat (HEPES, 2.5 g/l glocose) was RPMI-1640, the culture medium for SK-MES-1 was Eagle's MEM, the culture medium for 2-LMP and DY36T2 was IMEM, and the culture medium for SUM102 (5 ug/ml insulin) was F-12K. The cell viability was determined by ATPLite Assay Kit. If the IC50 value was lower than 62.5 ng/mL, the cells were treated with higher concentration of CTB006 in the second round of experiments to determine IC50 value.

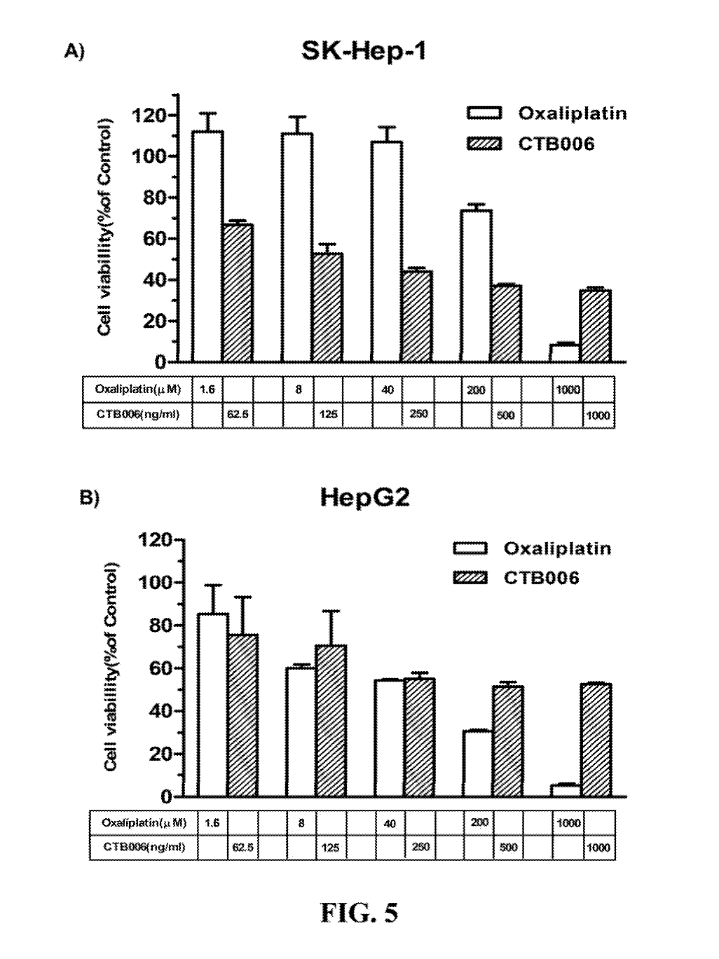

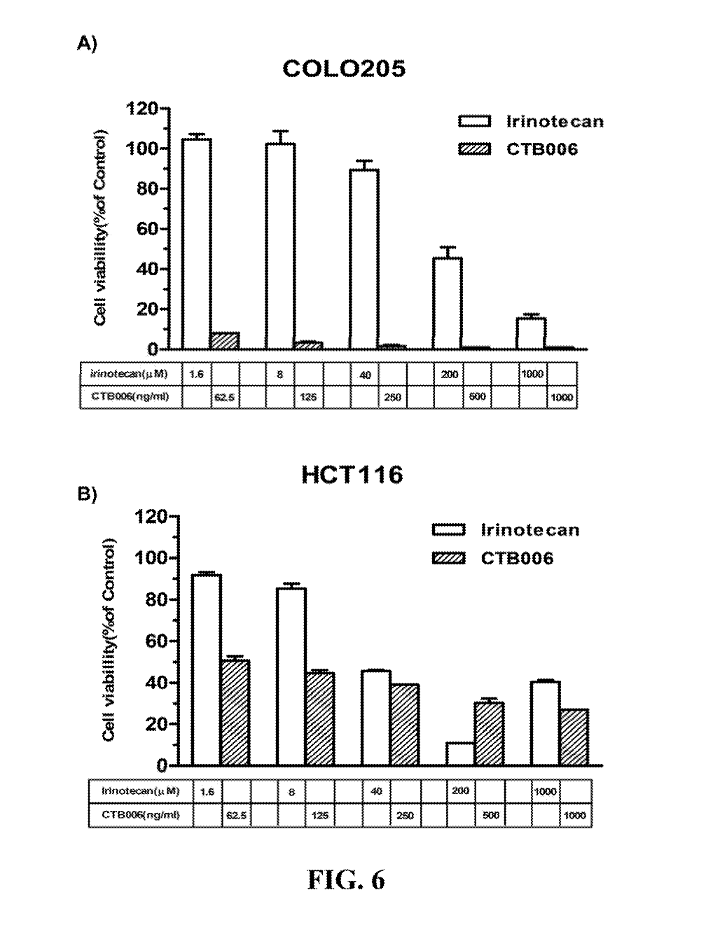

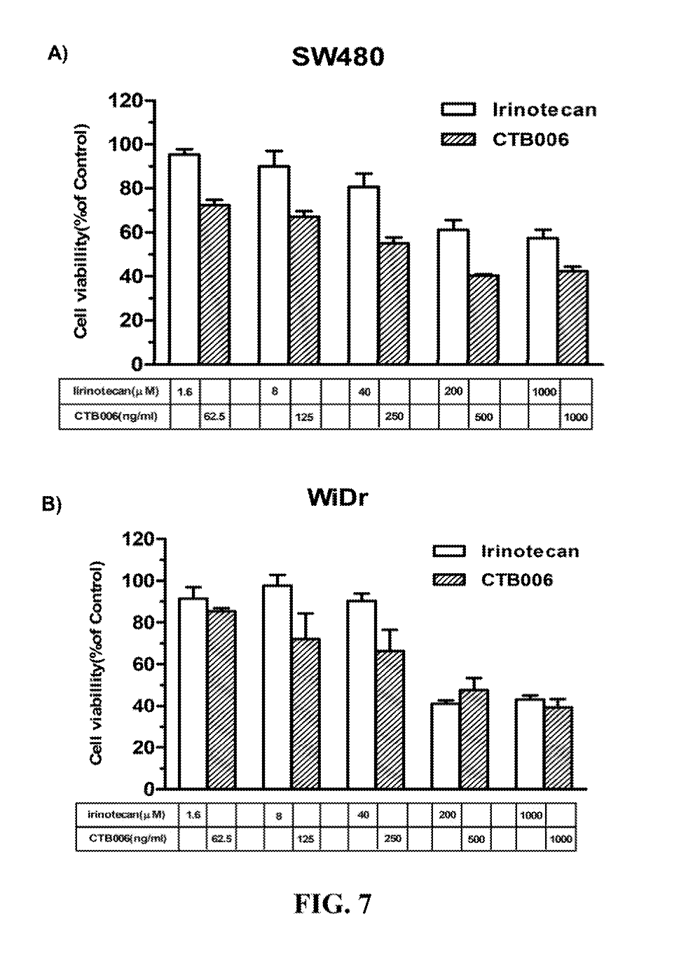

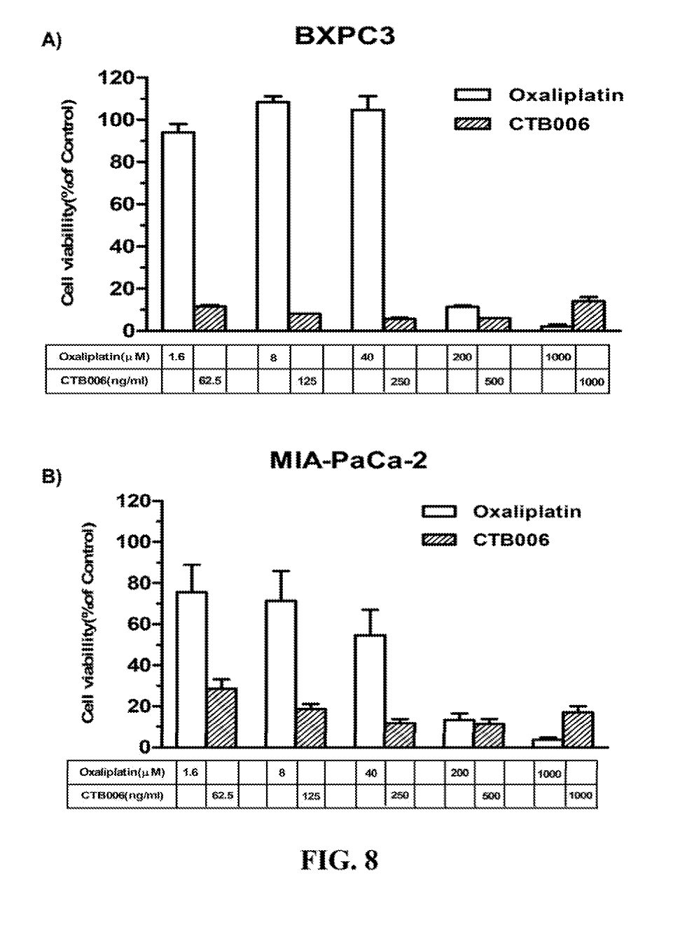

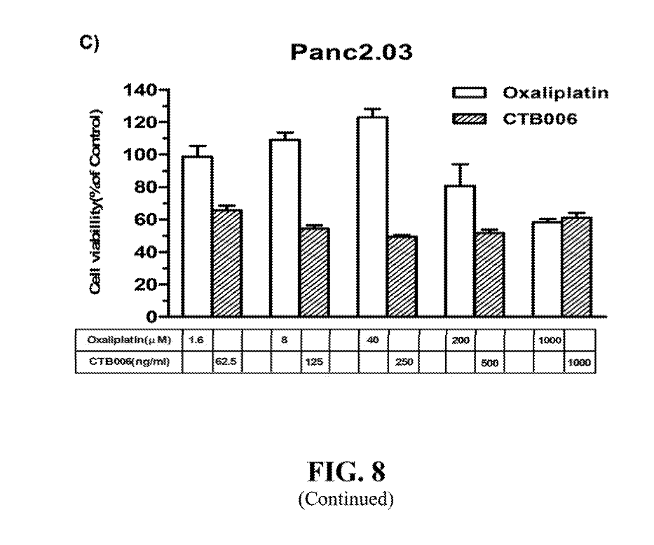

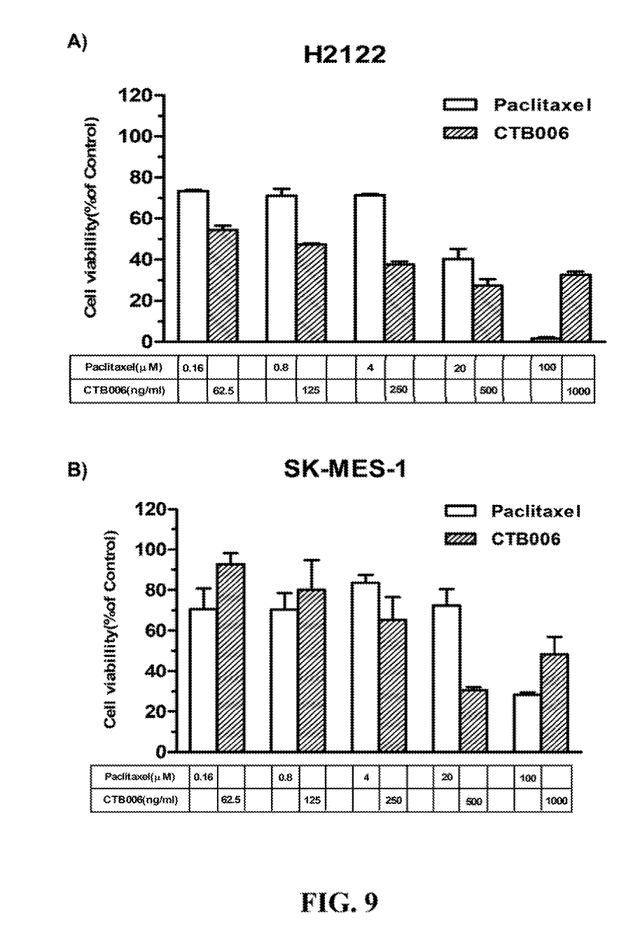

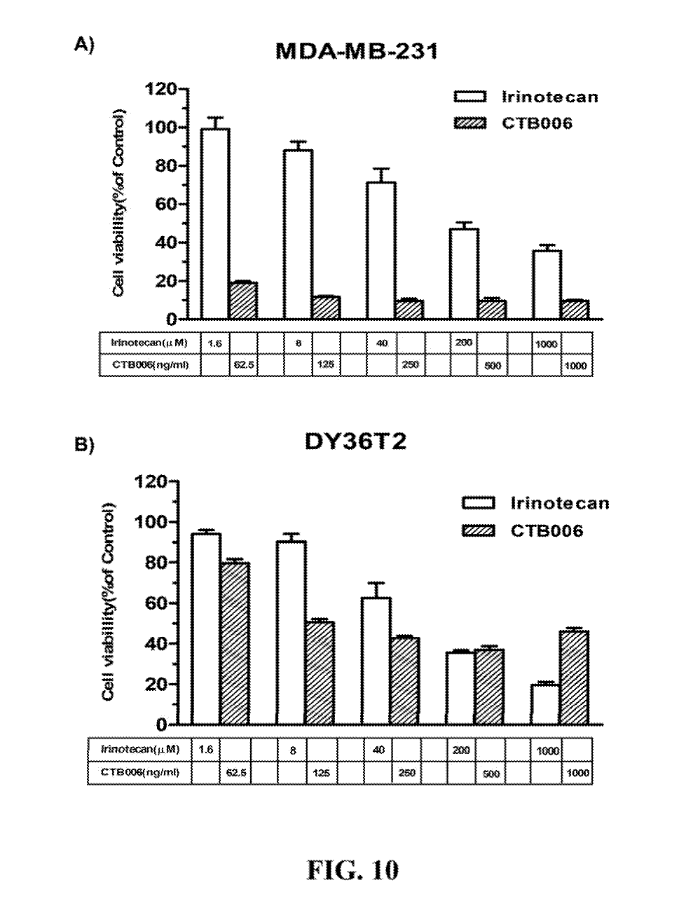

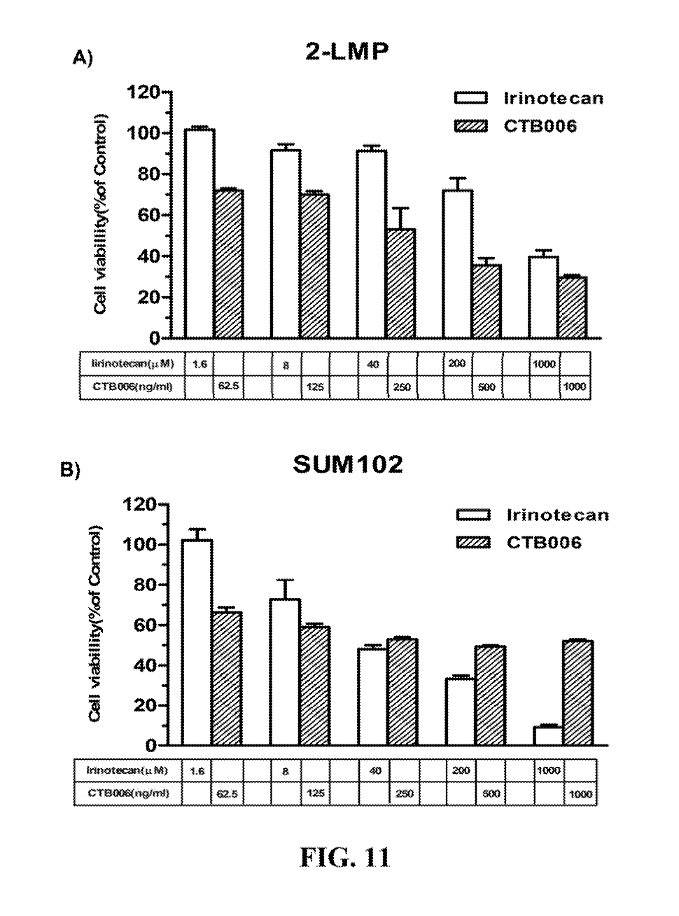

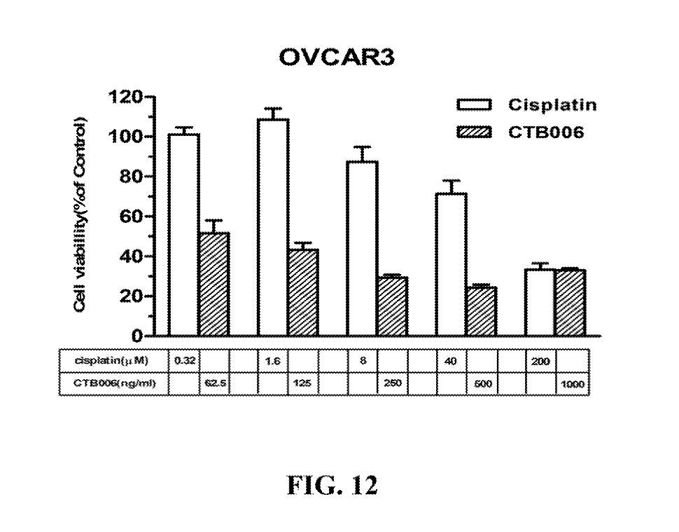

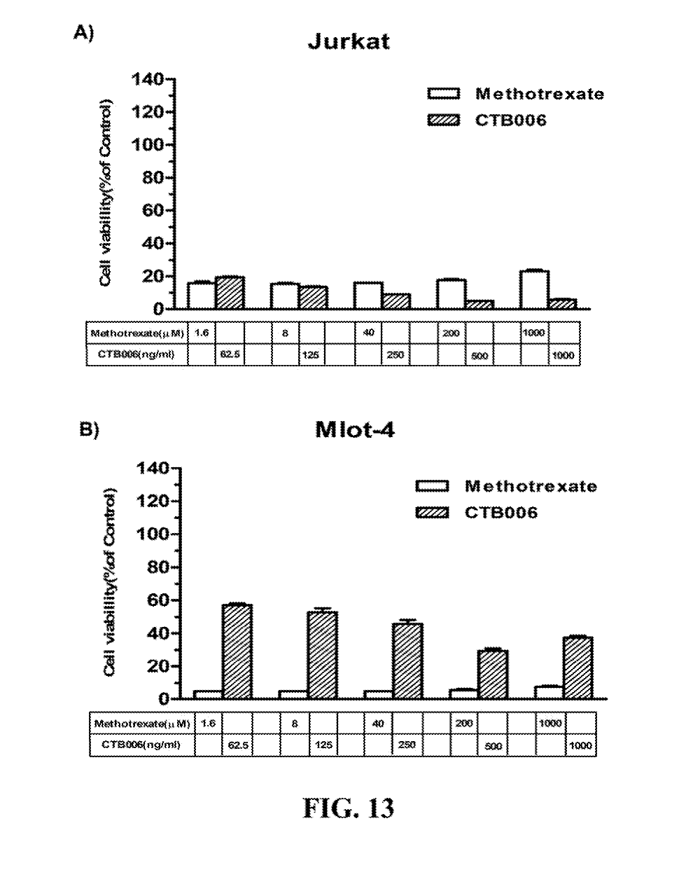

The results showed that HuCTB006 had anti-tumor activity on these eighteen human tumor cell lines, including liver cancer cell lines (SK-Hep-1 and HepG2 as shown in FIG. 5), colorectal cancer cell lines (COLO205, HCT116, SW480, and WiDr as shown in FIG. 6 & FIG. 7), pancreatic cancer cell lines (MIA-PaCa-2, BXPC3, and Panc2.03 as shown in FIG. 8), lung cancer cell lines (H2122 and SK-MES-1 as shown in FIG. 9), breast cancer cell lines (MDA-MB-231, 2-LMP, DY36T2, and SUM102 as shown in FIG. 10 & FIG. 11), ovarian cancer cell line (OVCAR3 as shown in FIG. 12) and acute T-lymphoblastic leukemia cell lines (Molt-4 and Jurkat as shown in FIG. 13).

Various tumor cell lines showed different sensitivity to CTB006, while some of them were more sensitive than others, such as COLO205, BXPC3, Jurkat and MDA-MB-231. The cell lines that showed more sensitivity to HuCTB006 have IC50 values almost close to 10 ng/mL as shown in Table 1, indicating that HuCTB006 has an effective and broad-spectrum of anti-tumor activity against various human tumor cell lines.

TABLE 1
IC50 Values Of HuCTB006 To Human Tumor Cell Lines

| Cell Lines | IC50 (ng/mL) |
|---|---|
| SK-Hep-1 | 197.1 |
| HepG2 | 499 |
| COLO205 | 12.68 |
| HCT116 | 67.07 |
| SW480 | 309.06 |
| WiDr | 515.17 |
| BXPC3 | 10.11 |
| MIA-PaCa-2 | 40.82 |
| Panc2.03 | 355.18 |
| H2122 | 90.92 |
| SK-MES-1 | 330.86 |
| OVCAR3 | 49.06 |
| MDA-MB-231 | 14.55 |
| DY36T2 | 206.46 |
| 2-LMP | 285.9 |
| SUM102 | 394.83 |
| Jurkat | 11.79 |
| Molt-4 | 108.72 |

### Example 3. Cytotoxic Effect of HuCTB006 and Chemotherapy Drug Combinations on Human Tumor Cell Lines

The coefficient of drug interaction (i.e., CDI) was used to analyze the synergetic effect of HuCTB006 and chemotherapy drug combination (Cao S S, et al., Potentiation of antimetabolite antitumor activity in vivo by dipyridamole and amphotericin B. Cancer Chemother Pharmacol 1989; 24: 181-186). CDI was calculated as follows: CDI=AB/A×B. According to the chemiluminescence of each group, AB was the ratio of the combination groups to control group; A or B was the ratio of the single agent groups to control group. CDI values less than, equal to or greater than 1 indicated that the drug combination was synergistic, additive or antagonistic, respectively. CDI less than 0.7 indicated that the drug combination was significantly synergistic.

3.1 Cytotoxic Effect of HuCTB006 and 5-FU Combination on Stomach Cancer Cell Line BGC823

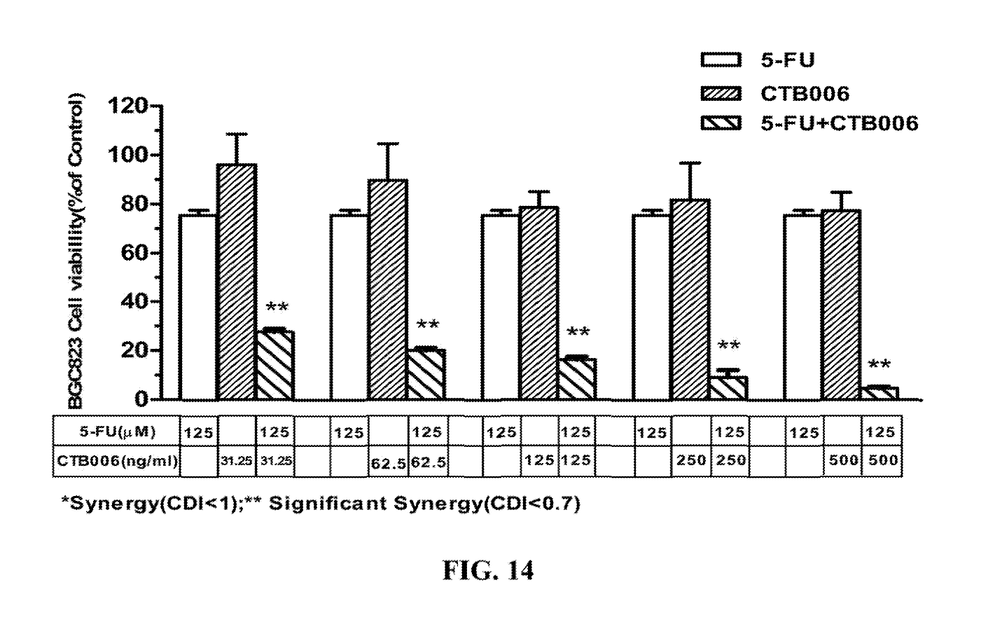

Stomach cancer cell line BGC823 was presented as a gift. The cells were cultured at 37° C. and 5% of CO₂, and in culture medium DMEM containing 10% of FBS. Cells were treated with different concentrations of CTB006 and 125 μM 5-FU as shown in Table 2 below. The CDI values were all less than 0.7 as shown in Table 2, indicating that the cytotoxic effect of HuCTB006 and 125 μM 5-FU on BGC823 cell line was significantly synergistic as shown in FIG. 14. The cells were treated with 5-FU for 24 hours, then different concentrations of HuCTB006 were added to the combined group, and the results were determined at 24 hours after the treatment.

TABLE 2
CDI of CT1B006 and 5-Fu combination on BGC823

| Concentration of CTB006 | Concentration of | |
|---|---|---|
| (ng/mL) | 5-Fu (μM) | CDI |
| 500 | 125 | 0.08** |
| 250 | 125 | 0.15** |
| 125 | 125 | 0.28** |
| 62.5 | 125 | 0.30** |
| 31.25 | 125 | 0.38** |

*Synergy (CDI <1);
**Significant synergy (CDI <0.7).

3.2 Cytotoxic Effect of HuCTB006 and 5-FU Combination on Pancreatic Cancer Cell Line Panc2.03

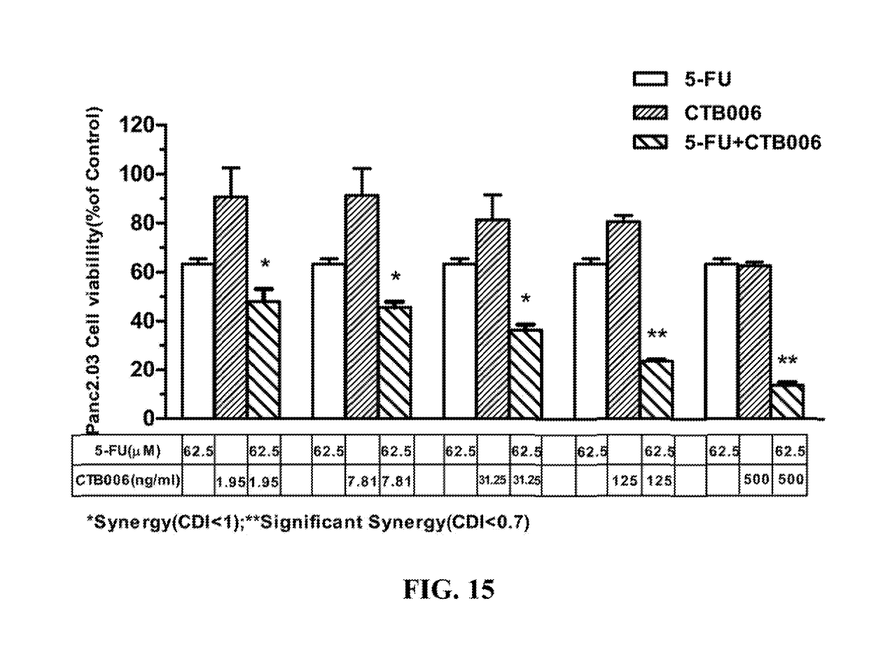

Pancreatic cancer cell line Panc2.03 cells were treated with different concentrations of HuCTB006 and 62.5 μM 5-Fu, and the CDI values were all less than 1 as shown in Table 3, indicating that the cytotoxic effect of HuCTB006 and 62.5 μM 5-FU on Panc2.03 cell line was synergistic. Further, the cytotoxic effect of HuCTB006 and 62.5 μM 5-FU on Panc2.03 cell line was significantly synergistic when the concentration of CTB006 was higher than 125 ng/mL as shown in FIG. 15. Panc 2.03 was cultured at 37° C. and 5% of CO₂, and in a culture medium RPMI 1640 containing 15% of FBS, 1 mM NaPyr, 1×MEM-NEAA, 10 mM human insulin, 10 mM HEPES. The cells were treated with 5-FU for 24 hours, then different concentrations of HuCTB006 were added to in the combined group, and the results were determined at 24 hours after the treatment.

TABLE 3
CDI of HuCTB006 and 5-Fu Combination on Panc2.03

| Conc. of HuCTB006 | Concentration of | |
|---|---|---|
| (ng/mL) | 5-FU (μM) | CDI |
| 500 | 62.5 | 0.35** |
| 125 | 62.5 | 0.47** |
| 31.25 | 62.5 | 0.72* |
| 7.81 | 62.5 | 0.79* |
| 1.95 | 62.5 | 0.84* |

*Synergy (CDI <1);
**Significant synergy (CDI <0.7).

3.3 Cytotoxic Effect of HuCTB006 and Paclitaxel Combination on Lung Cancer Cell Lines

3.3.1 A549 Cell Line

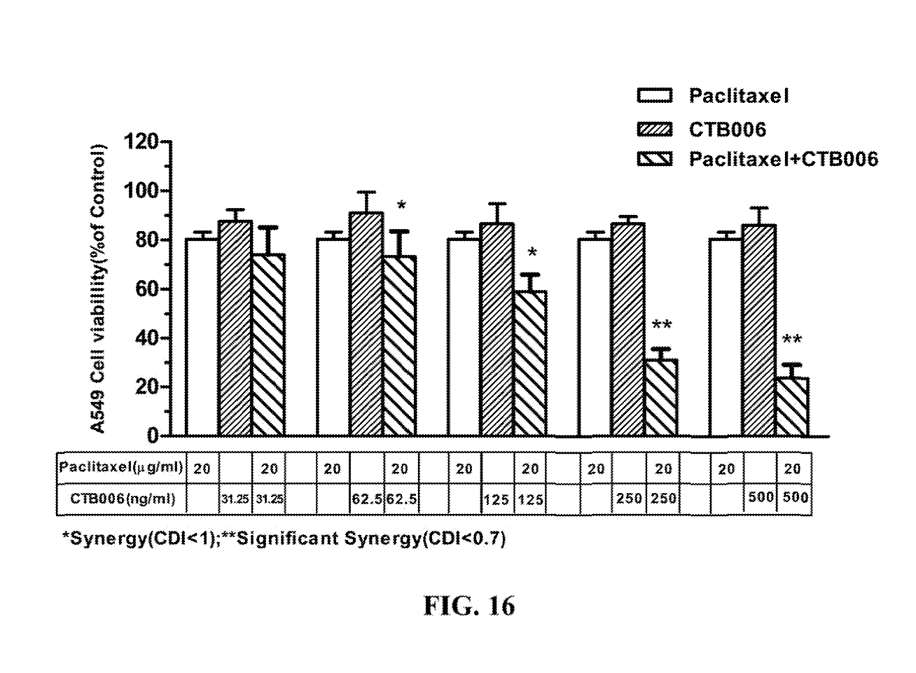

Lung cancer cell line A549 cells were treated with different concentrations of HuCTB006 and 20 μg/mL paclitaxel, and the CDI values were all less than 1 as shown in Table 4, indicating that the cytotoxic effect of HuCTB006 and 20 μg/mL paclitaxel on A549 cell line was synergistic. Further, the cytotoxic effect of HuCTB006 and 20 μg/mL paclitaxel on A549 cell line was significantly synergistic when the concentration of CTB006 was higher than 250 ng/mL as shown in FIG. 16. The cells were cultured at 37° C. and 5% of CO₂, and in culture medium RPMI 1640 containing 10% of FBS. The cells were treated with paclitaxel for 24 hours, then different concentrations of HuCTB006 were added to the combined group, the results were determined at 24 hours after the treatments.

TABLE 4
CDI of CTB006 and Paclitaxel Combination on A549

| Concentration of | Concentration of | |
|---|---|---|
| HuCTB006 (ng/mL) | Paclitaxel (μg/mL) | CDI |
| 500 | 20 | 0.32** |
| 250 | 20 | 0.41** |
| 125 | 20 | 0.79* |
| 62.5 | 20 | 0.93* |
| 31.25 | 20 | 0.99* |

*Synergy (CDI <1);
**Significant synergy (CDI <0.7).

3.3.2 H460 Cell Line

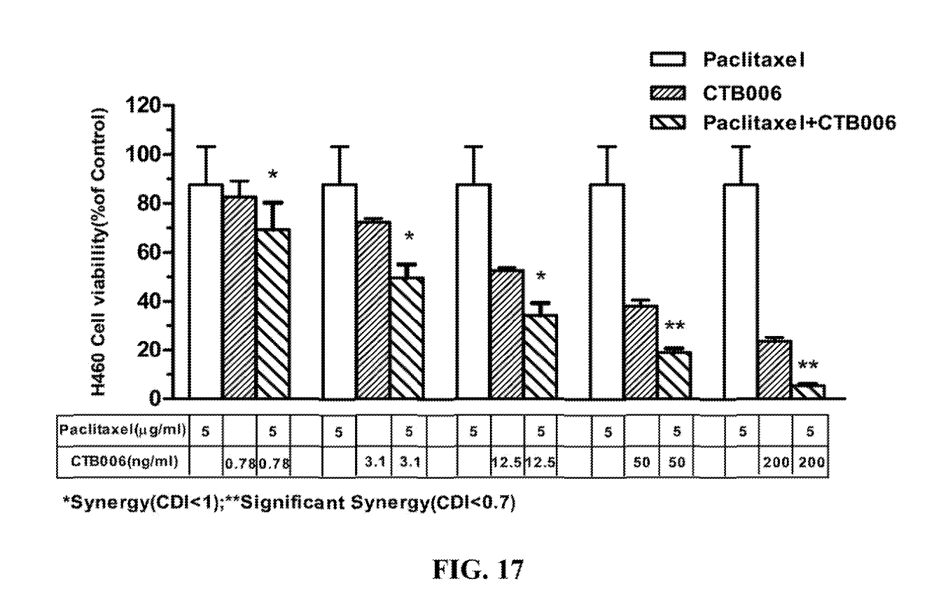

Lung cancer cell line H460 cells were treated with the combination of different concentrations of HuCTB006 and 5 μg/mL paclitaxel. The CDI values were all less than 1 as shown in Table 5, indicating that the cytotoxic effect of the combination of HuCTB006 and 5 μg/mL paclitaxel on H460 cells was synergistic. Further, the cytotoxic effect of the combination of HuCTB006 and 5 μg/mL paclitaxel on H460 cells was significantly synergistic when the concentration of HuCTB006 was higher than 50 ng/mL as shown in FIG. 17. The cells were cultured at 37° C. and 5% of CO₂, and in culture medium of RPMI 1640 containing 10% of FBS. The cells were treated with paclitaxel for 24 hours, then different concentrations of HuCTB006 were added to the combined group, the results were determined at 24 hours after the treatments.

TABLE 5
CDI of CTB006 and Paclitaxel Combination on H460

| Concentration of | Concentration of | |
|---|---|---|
| HuCTB006 (ng/mL) | Paclitaxel (μg/mL) | CDI |
| 200 | 5 | 0.25** |
| 50 | 5 | 0.57** |
| 12.5 | 5 | 0.74* |
| 3.1 | 5 | 0.78* |
| 0.78 | 5 | 0.96* |

*Synergy (CDI <1);
**Significant synergy (CDI <0.7).

3.4 Cytotoxic Effect of HuCTB006 and Chemotherapy Drug Combinations on Liver Cancer Cell Lines

3.4.1 Huh-7 Cell Line

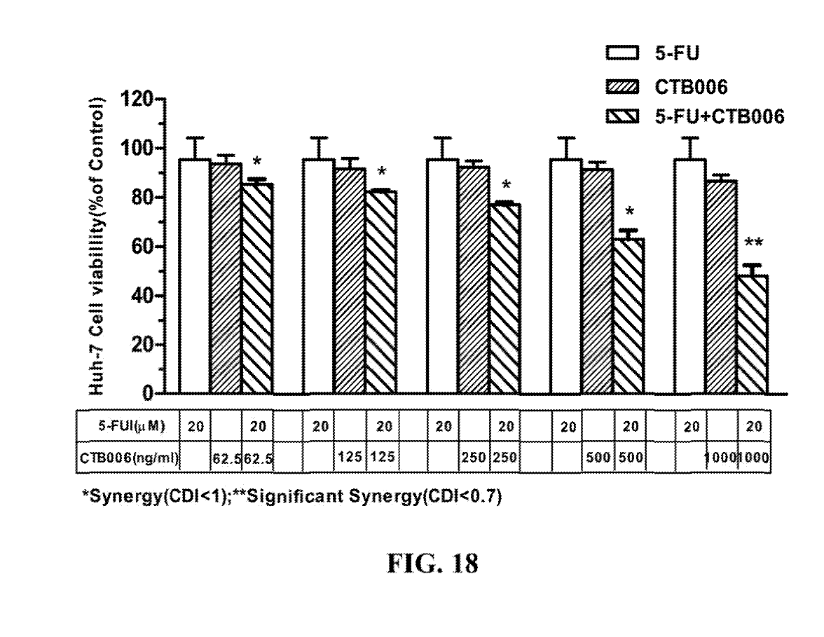

Liver cancer cell line Huh-7 cells were treated with the combination of different concentrations of HuCTB006 and 20 μM 5-Fu. The CDI values were all less than 1 as shown in Table 6, indicating that the cytotoxic effect of HuCTB006 and 20 μM 5-FU combination on Huh-7 cells was synergistic. Further, the cytotoxic effect of the combination of HuCTB006 and 20 μM 5-FU combination on Huh-7 cells was significantly synergistic when the concentration of HuCTB006 was 1000 ng/mL as shown in FIG. 18). The cells were cultured at 37° C. and 5% of CO₂, and in culture medium of DMEM containing 10% of FBS and 2 mM glutamine. The cells were treated with 5-FU for 24 hours, then different concentrations of HuCTB006 were added to the combined group, the results were determined at 24 hours after the treatments.

TABLE 6
CDI of HuCTB006 and 5-Fu Combination on Huh-7

| Concentration of | Concentration of | |
|---|---|---|
| HuCTB006 (ng/mL) | 5-FU (μM) | CDI |
| 1000 | 20 | 0.58** |
| 500 | 20 | 0.72* |
| 250 | 20 | 0.88* |
| 125 | 20 | 0.94* |
| 62.5 | 20 | 0.96* |

*Synergy (CDI <1);
**Significant synergy (CDI <0.7).

3.4.2 7402 Cell Line

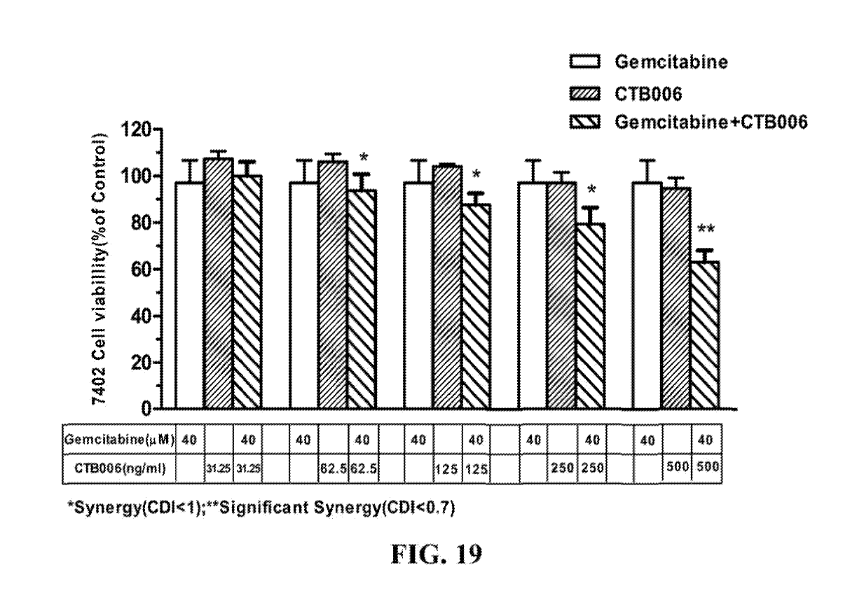

Liver cancer cell line 7402 cells were treated with the combination of different concentrations of HuCTB006 and 40 μM gemcitabine, and the CDI values were all less than 1 as shown in Table 7, indicating that the cytotoxic effect of the combination of HuCTB006 and 40 μM gemcitabine on 7402 cells was synergistic. Further, the cytotoxic effect of the combination of HuCTB006 and 40 μM gemcitabine on 7402 cells was significantly synergistic when the concentration of HuCTB006 was 500 ng/mL as shown in FIG. 19. The cells were cultured at 37° C. and 5% of CO₂, and in culture medium of DMEM containing 10% of FBS. The cells were treated with gemcitabine for 24 hours, then different concentrations of HuCTB006 were added to the combined group, the results were determined at 24 hours after the treatments.

TABLE 7
CDI of HuCTB006 and Gemcitabine Combination on 7402

| Concentration of | | |
|---|---|---|
| HuCTB006 | Concentration of Gemcitabine | |
| (ng/mL) | (μM) | CDI |
| 500 | 40 | 0.68** |
| 250 | 40 | 0.84* |
| 125 | 40 | 0.87* |
| 62.5 | 40 | 0.91* |
| 31.25 | 40 | 0.96* |

*Synergy (CDI <1);
**Significant synergy (CDI <0.7).

### Example 4. Cytotoxic Effect of HuCTB006 and Targeted Drug Combinations on Human Tumor Cell Lines

4.1 Cytotoxic Effect of HuCTB006 and Targeted Drug Combinations on Colorectal Cancer Cell Lines

4.1.1 SW480 Cell Line

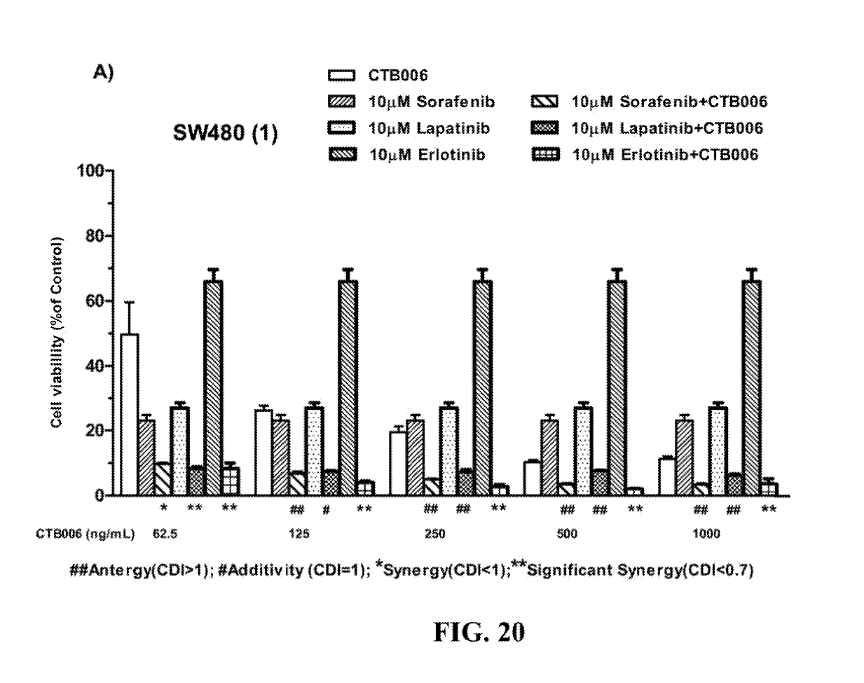

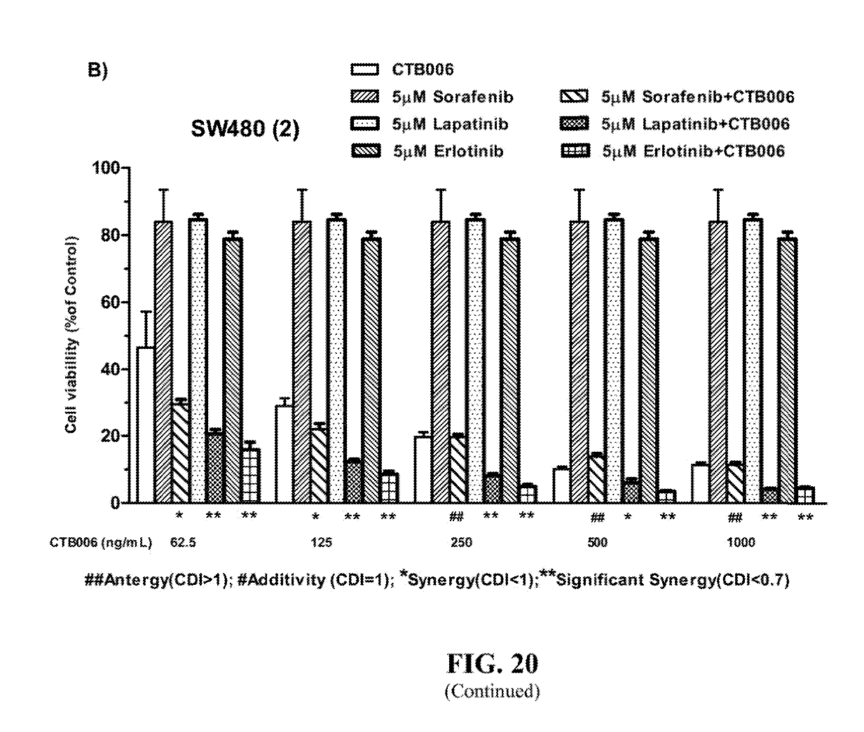

Colorectal cancer cell line SW480 cells were treated with the combination of different concentrations of HuCTB006, and 10 μM or 5 μM targeted drug combinations. The targeted drugs include Sorafenib, Lapatinib and Erlotinib. The CDI values are shown in Table 8. The CDI values were less than 0.7, when SW480 cells were treated with the combination of different concentrations of HuCTB006 and 10 μM Erlotinib, or 5 μM Erlotinib, or 5 μM Lapatinib, indicating that the cytotoxic effect of the combination of HuCTB006 and 10 μM Erlotinib, the combination of CTB006 and 5 μM Erlotinib, or the combination of HuCTB006 and 5 μM Lapatinib, on SW480 cells was significantly synergistic as shown in FIG. 20. Further, the cytotoxic effect of HuCTB006 and 10 μM Lapatinib on SW480 cells was significantly synergistic when the concentration of HuCTB006 was 62.5 ng/mL. Additionally, the cytotoxic effect of the combination of 62.5 ng/mL HuCTB006 and 10 μM Sorafenib, the combination of 62.5 ng/mL HuCTB006 and 5 μM Sorafenib, and the combination of 125 ng/mL HuCTB006 and 5 μM Sorafenib on SW480 cells were all synergistic since the CDI values were less than 1. The cells were cultured at 37° C. and 5% of CO₂, and in culture medium of DMEM containing 10% of FBS. The cells were treated with Sorafenib, Lapatinib or Erlotinib for 24 hours, then different concentrations of Hu HuCTB006 were added to the combined group, the results were determined at 24 hours after the treatments.

TABLE 8
CDI ofand Targeted Drug Combinations on SW480

| Concentration of | CDI | | | | | |
|---|---|---|---|---|---|---|
| HuCTB006 | Sorafenib | | Lapatinib | | Erlotinib | |
| (ng/mL) | 10 μM | 5 μM | 10 μM | 5 μM | 10 μM | 5 μM |
| 1000 | 1.31 | 1.20 | 2.07 | 0.43** | 0.50** | 0.51** |
| 500 | 1.50 | 1.64 | 2.75 | 0.71* | 0.31** | 0.43** |
| 250 | 1.11 | 1.19 | 1.37 | 0.49** | 0.21** | 0.32** |
| 125 | 1.12 | 0.90* | 1.04 | 0.50** | 0.23** | 0.38** |
| 62.5 | 0.85* | 0.75* | 0.62** | 0.52** | 0.25** | 0.43** |

*Synergy (CDI <1);
**Significant synergy (CDI <0.7).

4.1.2 Widr Cell Line

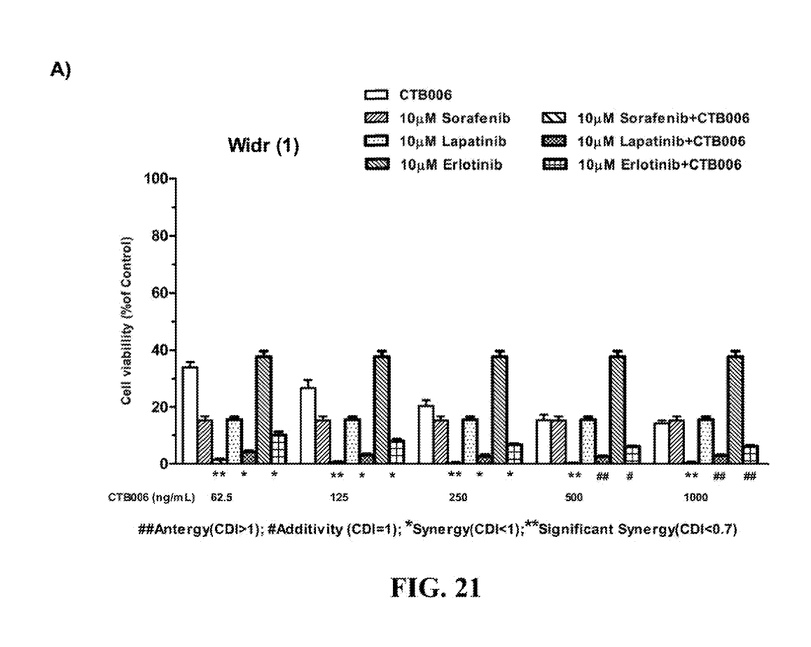

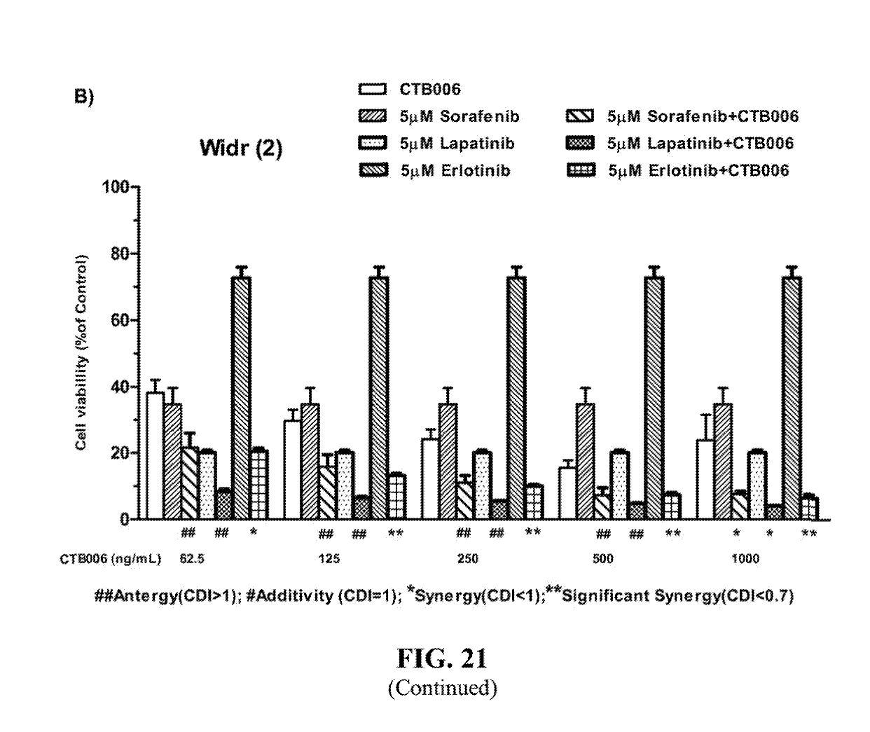

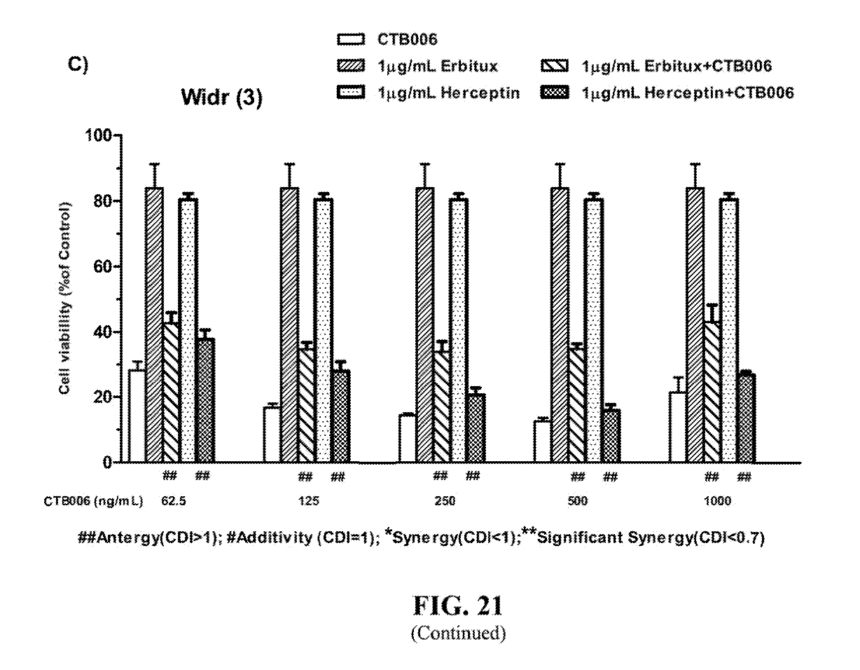

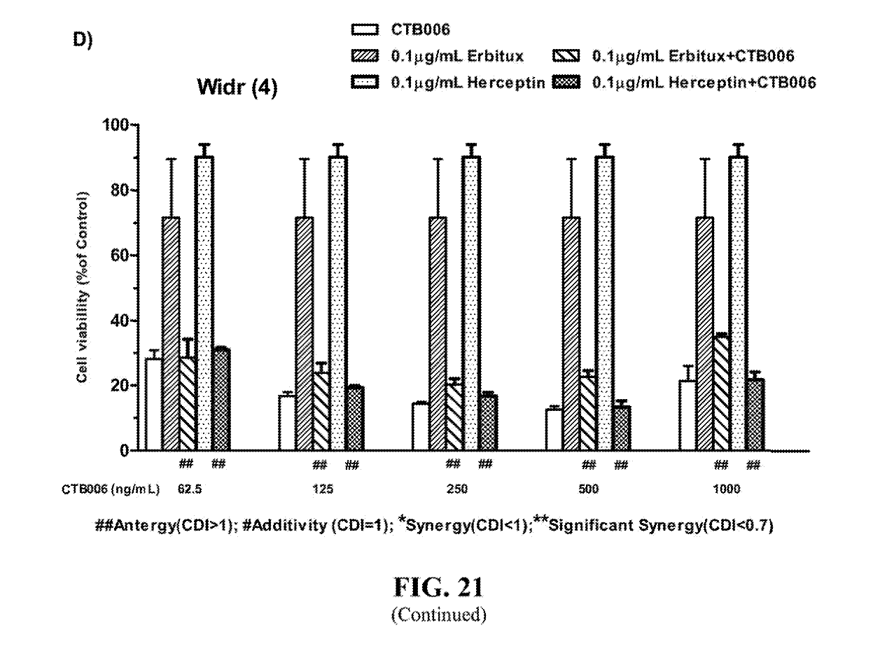

Colorectal cancer cell line Widr cells were treated with the combination of different concentrations of HuCTB006 and targeted drugs, including Sorafenib, Lapatinib, Erlotinib, Erbitux and Herceptin. The CDI values were shown in Table 9. The CDI values were less than 0.7 when Widr cells were treated with the combination of different concentrations of HuCTB006 and 10 μM Sorafenib or 5 μM Erlotinib, indicating that the cytotoxic effect of the combination of HuCTB006 and 10 μM Sorafenib or the combination of HuCTB006 and 5 μM Erlotinib on Widr cells was significantly synergistic as shown in FIG. 21. Further, the cytotoxic effect of the combination of HuCTB006 and 5 μM Sorafenib or the combination of HuCTB006 and 5 μM Lapatinib on Widr cells was synergistic when the concentration of HuCTB006 was 1000 ng/mL. Additionally, the cytotoxic effect of 250 ng/mL, 125 ng/mL and 62.5 ng/mL HuCTB006 and 10 μM Lapatinib or 10 μM Erlotinib combinations on Widr cells was synergistic since the CDI values were less than 1. The cytotoxic effect of HuCTB006 and Erbitux or CTB006 and Herceptin combinations on Widr cells was all antergy for the CDI values were higher than 1. The cells were cultured at 37° C. and 5% of CO₂, and in culture medium of DMEM containing 10% of FBS and 1×NEAA. The cells were treated with Sorafenib, Lapatinib, Erlotinib, Erbitux or Herceptin for 24 hours, then different concentrations of CTB006 were added to the combined group, the results were determined at 24 hours after the treatments.

TABLE 9
CDI of HuCTB006 and Targeted Drug Combinations on Widr

| Conc. of | CDI | | | | | | | | | |
|---|---|---|---|---|---|---|---|---|---|---|
| HuCTB006 | Sorafenib | | Lapatinib | | Erlotinib | | Erbitux | | Herceptin | |
| (ng/mL) | 10 μM | 5 μM | 10 μM | 5 μM | 10 μM | 5 μM | 1 μg/m | 0.1 μg/m | 1 μg/m | 0.1 μg/m |
| 1000 | 0.16** | 0.92* | 1.30 | 0.85* | 1.14 | 0.37** | 2.41 | 2.29 | 1.57 | 1.13 |
| 500 | 0.11** | 1.36 | 1.06 | 1.54 | 1.04 | 0.66** | 3.30 | 2.52 | 1.57 | 1.18 |
| 250 | 0.11** | 1.32 | 0.84* | 1.14 | 0.87* | 0.57** | 2.81 | 1.97 | 1.78 | 1.29 |
| 125 | 0.14** | 1.54 | 0.75* | 1.09 | 0.79* | 0.61** | 2.46 | 1.99 | 2.07 | 1.27 |
| 62.5 | 0.26** | 1.62 | 0.78* | 1.10 | 0.78* | 0.73* | 1.81 | 1.42 | 1.67 | 1.22 |

*Synergy (CDI <1);
**Significant synergy (CDI <0.7).

4.2 Cytotoxic Effect of HuCTB006 and Targeted Drug Combinations on Stomach Cancer Cell Lines

4.2.1 BGC823 Cell Line

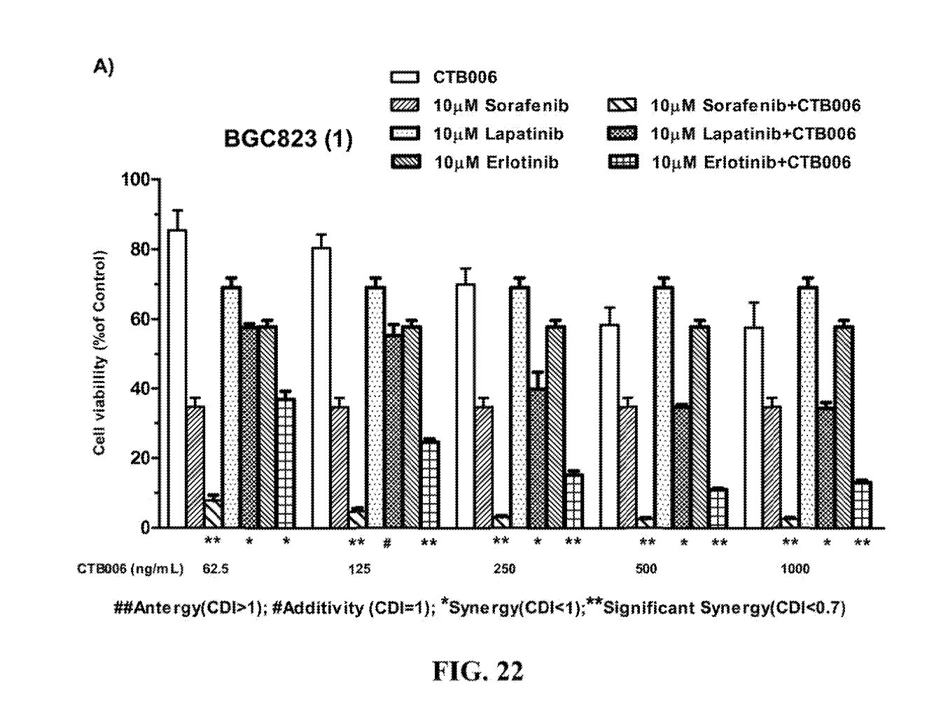

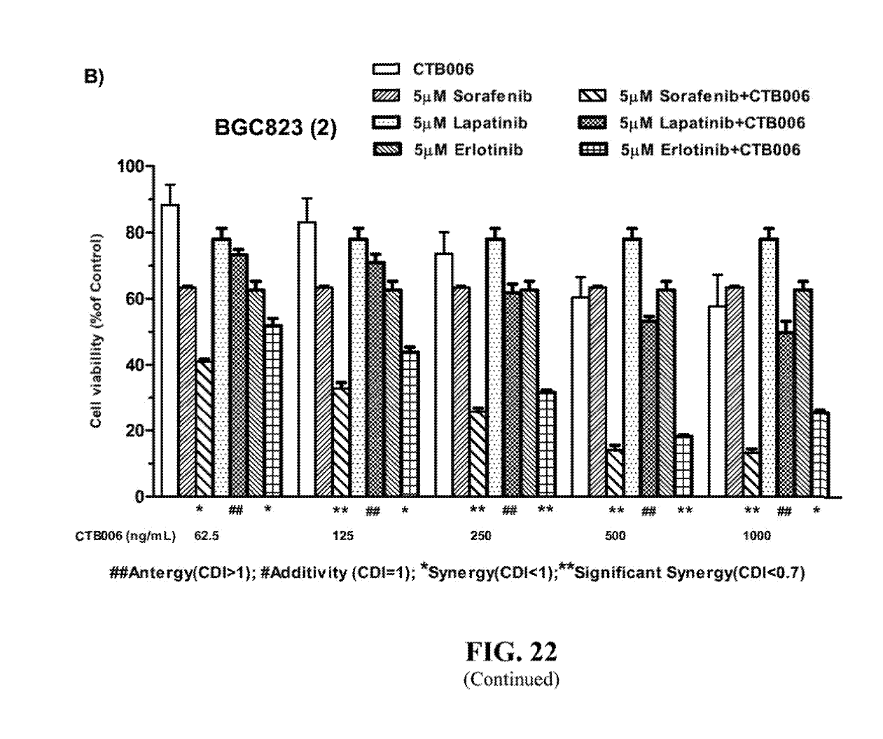

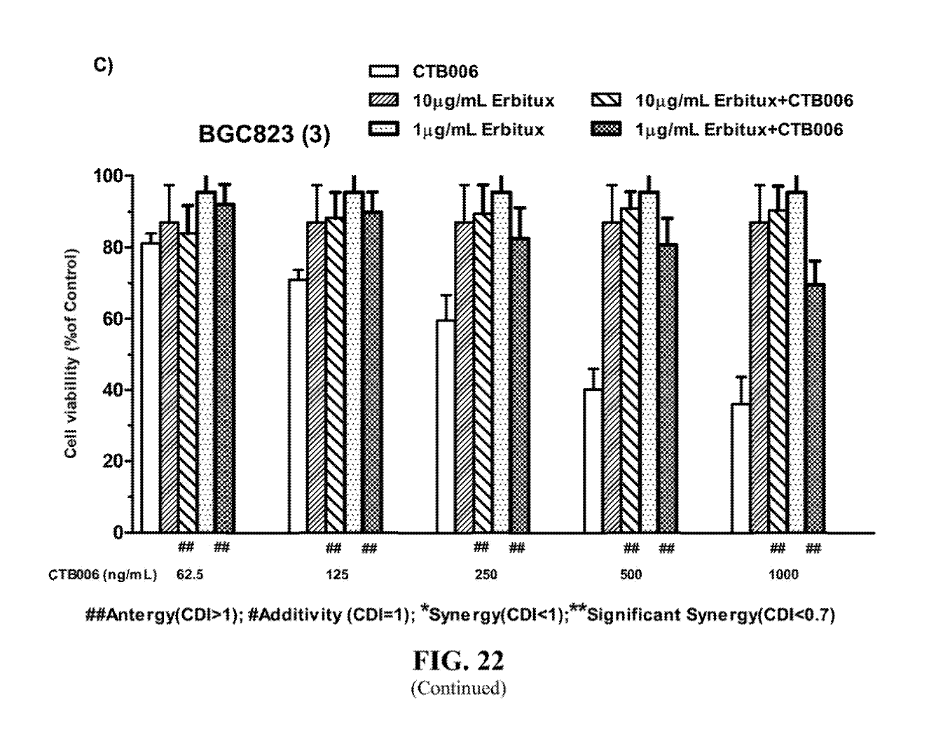

Stomach cancer cell line BGC823 cells were treated with the combination of different concentrations of HuCTB006 and targeted drugs, including Sorafenib, Lapatinib, Erlotinib and Erbitux. The CDI values were shown in Table 10. The CDI values were less than 0.7, when BGC823 cells were treated with different concentrations of HuCTB006 and Sorafenib or Erlotinib combinations, indicating that the cytotoxic effect of HuCTB006 and Sorafenib or Erlotinib combinations on BGC823 cells was significantly synergistic as shown in FIG. 22. Further, the cytotoxic effect of HuCTB006 and 10 μM Lapatinib on BGC823 cells was synergistic except for the combination of 125 ng/mL CTB006 and 10 μM Lapatinib. Additionally, the cytotoxic effect of the combinations of HuCTB006 and Erbitux on BGC823 cells was all antergy for the CDI values were higher than 1. The cells were cultured at 37° C. and 5% of CO₂, and in culture medium of DMEM containing 10% of FBS. The cells were treated with Sorafenib, Lapatinib, Erlotinib, or Erbitux for 24 hours, then different concentrations of HuCTB006 were added to the combined group, the results were determined at 24 hours after the treatments.

TABLE 10
CDI of HuCTB006 and Targeted Drug Combinations on BGC823

| Concentration | CDI | | | | | | | |
|---|---|---|---|---|---|---|---|---|
| of HuCTB006 | Sorafenib | | Lapatinib | | Erlotinib | | Erbitux | |
| (ng/mL) | 10 μM | 5 μM | 10 μM | 5 μM | 10 μM | 5 μM | 10 μg/mL | 1 μg/mL |
| 1000 | 0.13** | 0.36** | 0.87* | 1.11 | 0.39** | 0.71* | 2.87 | 2.01 |
| 500 | 0.12** | 0.37** | 0.86* | 1.13 | 0.33** | 0.48** | 2.60 | 2.10 |
| 250 | 0.13** | 0.55** | 0.83* | 1.08 | 0.38** | 0.68** | 1.73 | 1.45 |
| 125 | 0.17** | 0.62** | 1.00 | 1.09 | 0.53** | 0.84* | 1.43 | 1.33 |
| 62.5 | 0.26** | 0.73* | 0.98* | 1.06 | 0.75* | 0.94* | 1.19 | 1.19 |

*Synergy (CDI <1);
**Significant synergy (CDI <0.7).

4.2.2 NUGC3 Cell Line

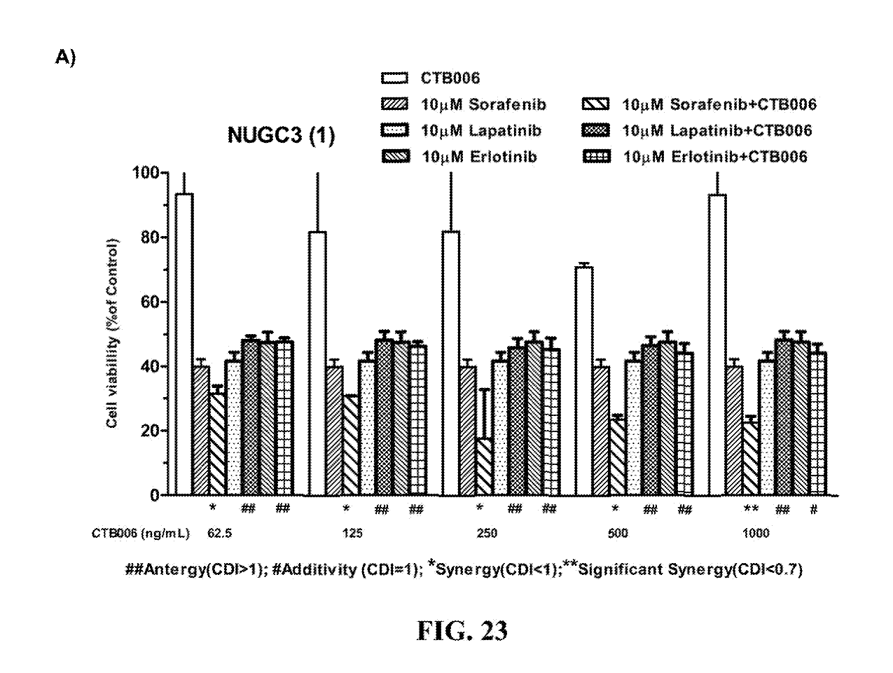

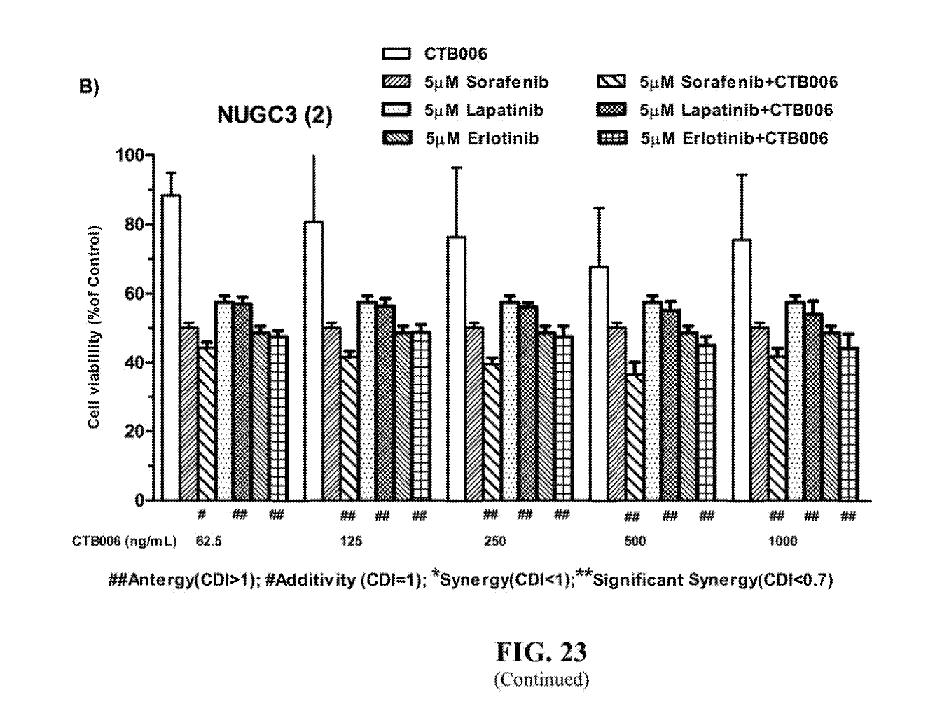

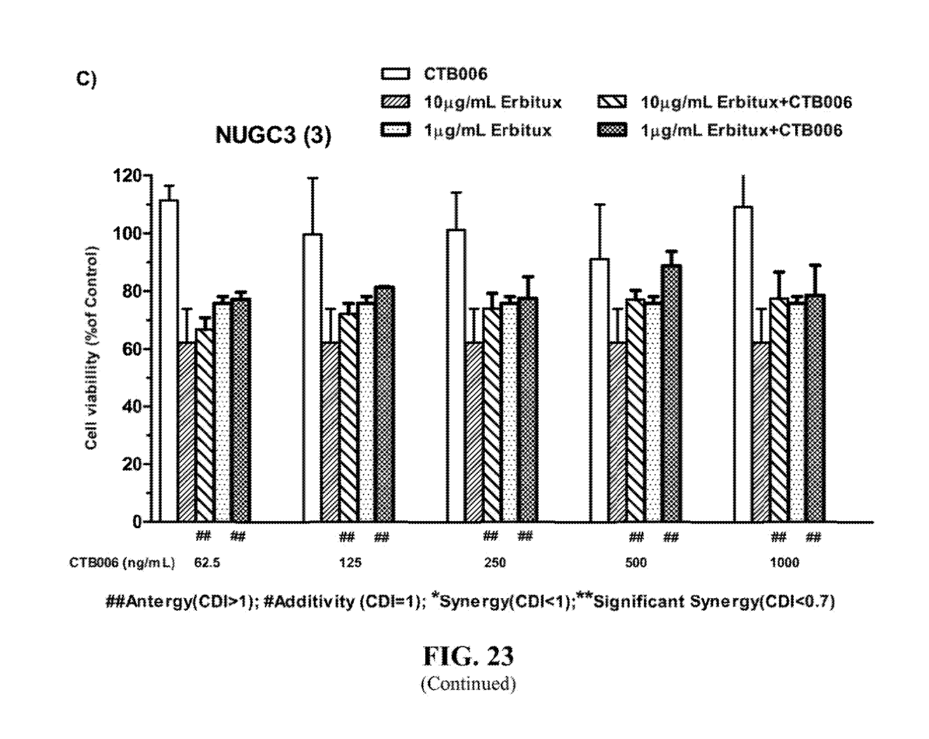

Stomach cancer cell line NUGC3 cells were treated with the combination of different concentrations of HuCTB006 and targeted drugs, including Sorafenib, Lapatinib, Erlotinib and Erbitux, and the CDI values were shown in Table 11. The CDI values were less than 1 when NUGC3 cells were treated with the combination of different concentrations of HuCTB006 and 10 μM Sorafenib, indicated that the cytotoxic effect of HuCTB006 and Sorafenib combination on NUGC3 cells was synergistic or significantly synergistic as shown in FIG. 23. Additionally, the cytotoxic effect of HuCTB006 and other targeted drug combinations on NUGC3 cells was all antergy for the CDI values were higher than 1. The cells were cultured at 37° C. and 5% of CO₂, and in culture medium of DMEM containing 10% of FBS. The cells were treated with Sorafenib, Lapatinib, Erlotinib, or Erbitux for 24 hours, then different concentrations of HuCTB006 were added to the combined group, the results were determined at 24 hours after the treatments.

TABLE 11
CDI of HuCTB006 and Targeted Drug Combinations on NUGC3

| Concentration | CDI | | | | | | | |
|---|---|---|---|---|---|---|---|---|
| of HuCTB006 | Sorafenib | | Lapatinib | | Erlotinib | | Erbitux | |
| (ng/mL) | 10 μM | 5 μM | 10 μM | 5 μM | 10 μM | 5 μM | 10 μg/mL | 1 μg/mL |
| 1000 | 0.61** | 1.10 | 1.25 | 1.25 | 1.00 | 1.20 | 1.14 | 0.95 |
| 500 | 0.83* | 1.08 | 1.58 | 1.42 | 1.31 | 1.37 | 1.36 | 1.29 |
| 250 | 0.81* | 1.03 | 1.35 | 1.28 | 1.17 | 1.28 | 1.18 | 1.01 |
| 125 | 0.94* | 1.03 | 1.42 | 1.22 | 1.19 | 1.24 | 1.16 | 1.08 |
| 62.5 | 0.85* | 1.00 | 1.24 | 1.12 | 1.07 | 1.10 | 0.96 | 0.92 |

*Synergy (CDI <1);
**Significant synergy (CDI <0.7).

4.3 H460 Cell Line

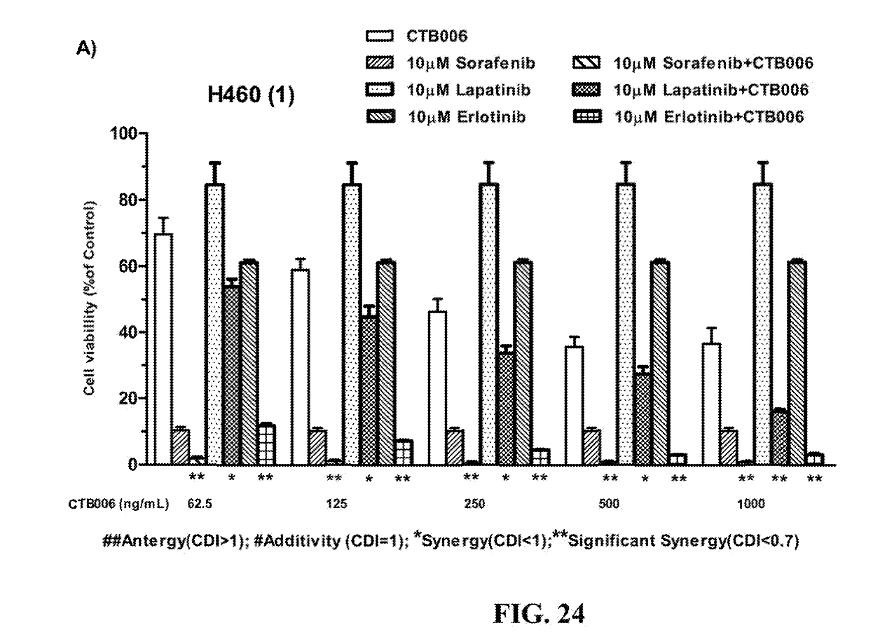

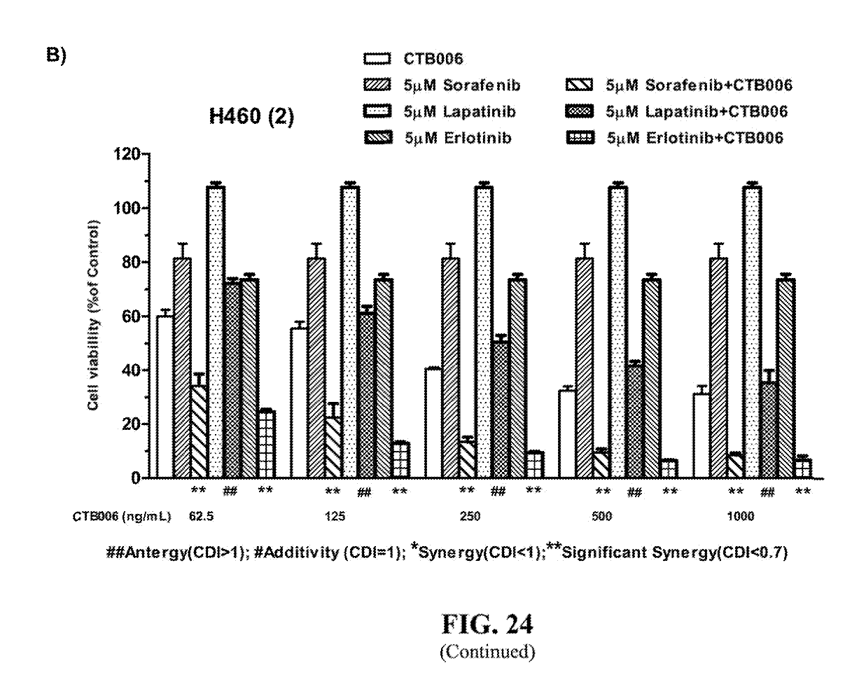

Lung cancer cell line H460 cells were treated with the combination of different concentrations of HuCTB006 and targeted drugs, including Sorafenib, Lapatinib, and Erlotinib, and the CDI values were shown in Table 12. The CDI values were all less than 0.7, when H460 cells were treated with the combinations of HuCTB006 and Sorafenib or HuCTB006 and Erlotinib, indicating that the cytotoxic effect of CTB006 and Sorafenib or Erlotinib combinations on H460 cells was significantly synergistic as shown in FIG. 24. Further, the cytotoxic effect of HuCTB006 and 10 μM Lapatinib on H460 cells was synergistic, or even significantly synergistic when the concentration of HuCTB006 was 1000 ng/mL. The cells were cultured at 37° C. and 5% of CO₂, and in culture medium of RPMI 1640 containing 10% of FBS. The cells were treated with Sorafenib, Lapatinib, or Erlotinib for 24 hours, then different concentrations of CTB006 were added to the combined group, the results were determined at 24 hours after the treatments.

TABLE 12
CDI of HuCTB006 and Targeted Drug Combinations on H460

| Concentration | CDI | | | | | |
|---|---|---|---|---|---|---|
| of HuCTB006 | Sorafenib | | Lapatinib | | Erlotinib | |
| (ng/mL) | 10 μM | 5 μM | 10 μM | 5 μM | 10 μM | 5 μM |
| 1000 | 0.24** | 0.34** | 0.52** | 1.05 | 0.14** | 0.29** |
| 500 | 0.19** | 0.36** | 0.91* | 1.19 | 0.14** | 0.27** |
| 250 | 0.14** | 0.41** | 0.86* | 1.16 | 0.16** | 0.32** |
| 125 | 0.18** | 0.50** | 0.90* | 1.02 | 0.20** | 0.32** |
| 62.5 | 0.27** | 0.70** | 0.91* | 1.12 | 0.28** | 0.55** |

*Synergy (CDI <1);
**Significant synergy (CDI <0.7).

4.4 Cytotoxic Effect of HuCTB006 and Targeted Drug Combinations on Breast Cancer Cell Lines

4.4.1 SK-BR3 Cell Line

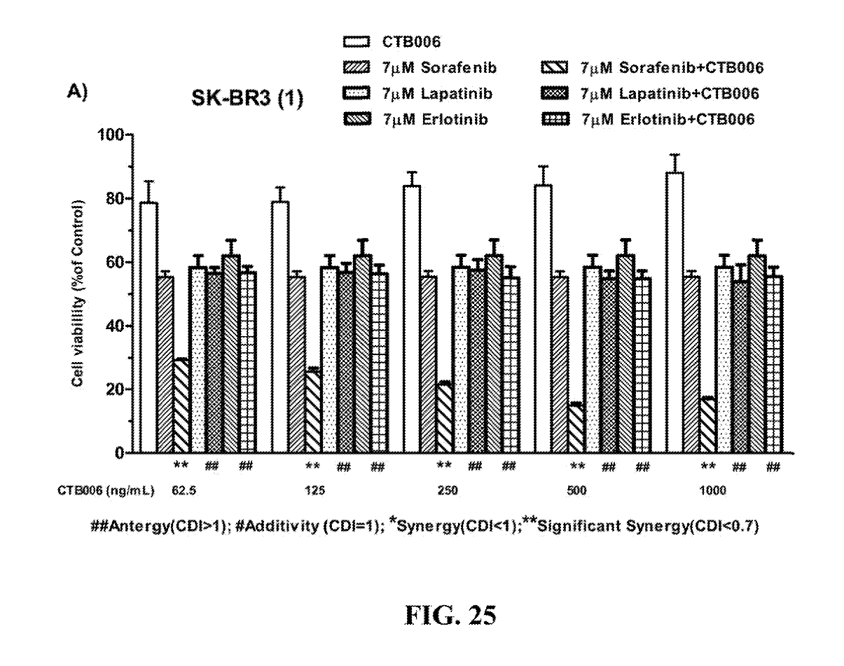

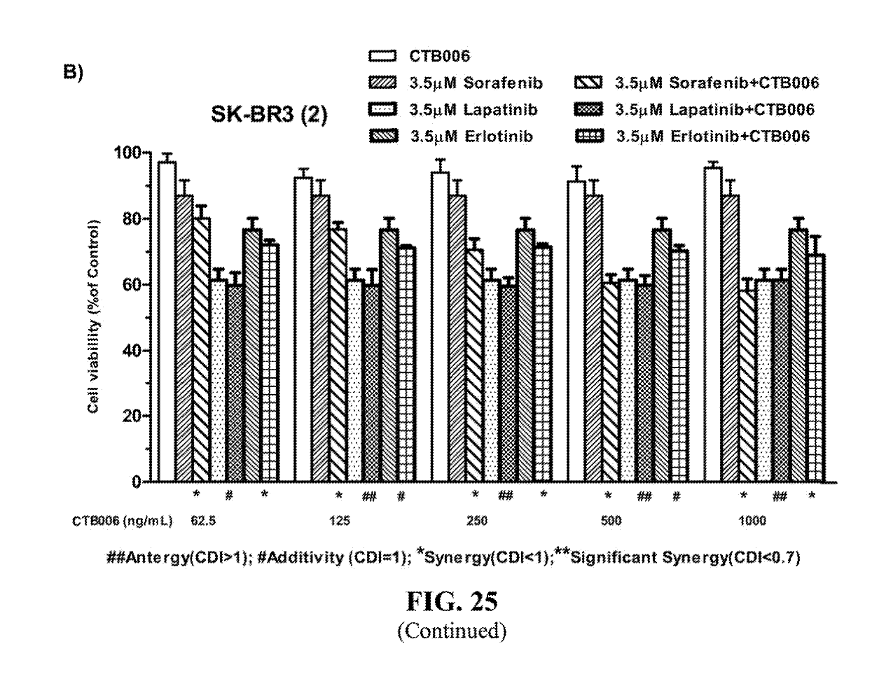

Breast cancer cell line SK-BR3 cells were treated with the combinations of different concentrations of HuCTB006 and 10 μM or 5 μM targeted drugs, including Sorafenib, Lapatinib and Erlotinib. The CDI values were shown in Table 13. The CDI values were less than 0.7, when SK-BR3 cells were treated with different concentration of HuCTB006 and 10 μM Sorafenib combinations, and the CDI values were less than 1, when SK-BR3 cells were treated with different concentrations of HuCTB006 and 5 μM Sorafenib combinations, indicating that the cytotoxic effect of HuCTB006 and 10 μM or 5 μM Sorafenib combinations on SK-BR3 cells was significantly synergistic as shown in FIG. 25. Further, the cytotoxic effect of the combinations of HuCTB006 and 10 μM Erlotinib on SK-BR3 cells was synergistic, when the concentration of HuCTB006 was 1000, 250 and 62.5 ng/mL. The cytotoxic effect of HuCTB006 and Lapatinib combinations on SK-BR3 cells was antergy for the CDI values were higher than 1. The cells were cultured at 37° C. and 5% of CO₂, and in culture medium of McCoy's 5a containing 10% of FBS. The cells were treated with Sorafenib, Lapatinib, or Erlotinib for 24 hours, then different concentrations of HuCTB006 were added to the combined group, the results were determined at 24 hours after the treatments.

TABLE 13
CDI of HuCTB006 and Targeted Drug Combinations on SK-BR3

| Concentration | CDI | | | | | |
|---|---|---|---|---|---|---|
| of HuCTB006 | Sorafenib | | Lapatinib | | Erlotinib | |
| (ng/mL) | 10 μM | 5 μM | 10 μM | 5 μM | 10 μM | 5 μM |
| 1000 | 0.35** | 0.70* | 1.05 | 1.05 | 1.02 | 0.94* |
| 500 | 0.32** | 0.76* | 1.11 | 1.07 | 1.05 | 1.00 |
| 250 | 0.46** | 0.86* | 1.17 | 1.03 | 1.06 | 0.99* |
| 125 | 0.58** | 0.96* | 1.23 | 1.05 | 1.15 | 1.01 |
| 62.5 | 0.67** | 0.95* | 1.23 | 1.00 | 1.16 | 0.97* |

*Synergy (CDI <1);
**Significant synergy (CDI <0.7).

4.4.2 SUM102 Cell Line

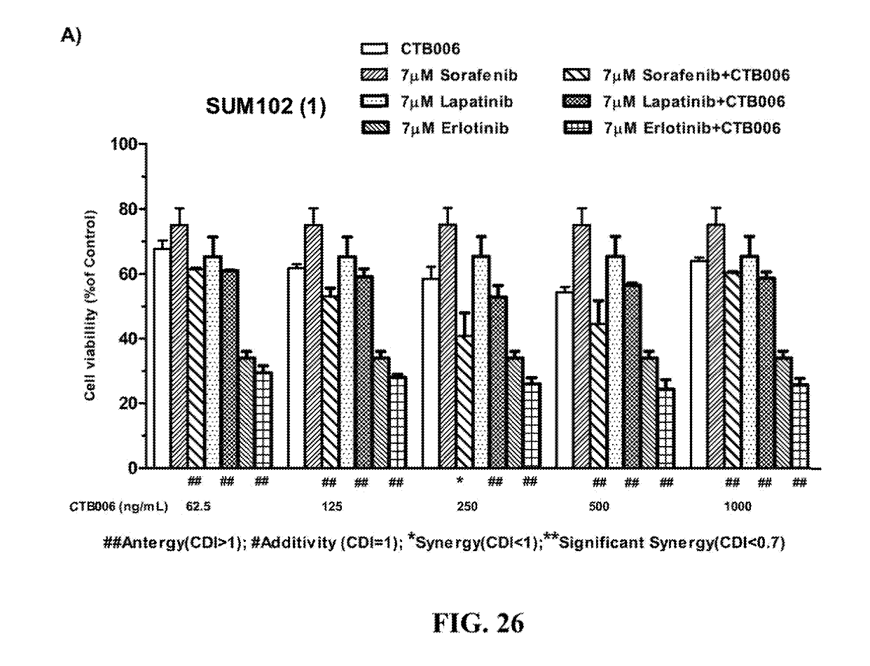

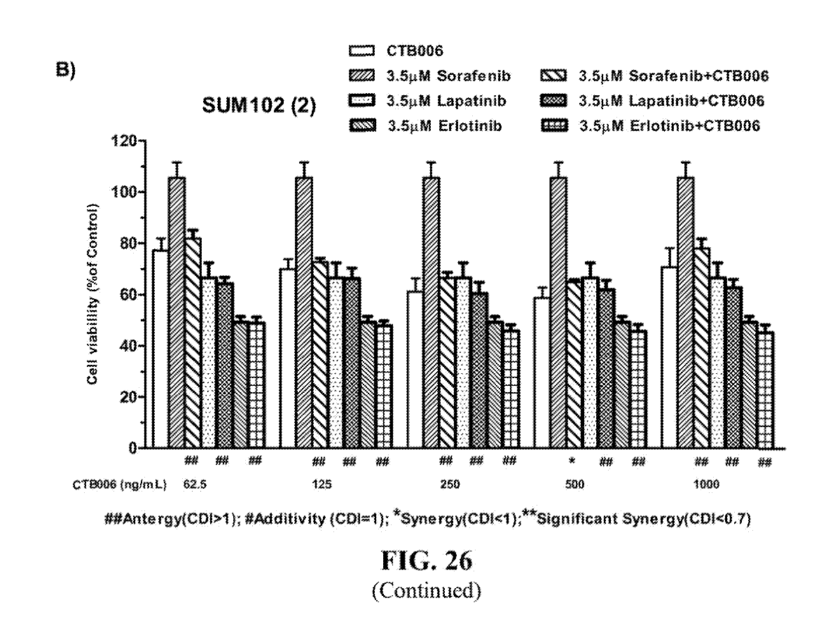

Breast cancer cell line SUM102 cells were treated with the combination of different concentrations of HuCTB006, and 10 μM or 5 μM targeted drugs, including Sorafenib, Lapatinib and Erlotinib. The CDI values were shown in Table 14. The CDI values were all higher than 1, when SUM102 cells were treated with the combination of different concentrations of HuCTB006 and Lapatinib or Erlotinib, indicating that the cytotoxic effect of HuCTB006 and Lapatinib or HuCTB006 and Erlotinib combinations on SUM102 cells was antergy as shown in FIG. 26. The cytotoxic effect of HuCTB006 and Sorafenib combinations on SUM102 cells was also antergy except for the combination of 250 ng/mL HuCTB006 and 10 μM Sorafenib, and the combination of 125 ng/mL HuCTB006 and 5 μM Sorafenib. The cells were cultured at 37° C. and 5% of CO₂, and in culture medium of F-12K containing 10% of FBS and 5 μg/ml insulin. The cells were treated with Sorafenib, Lapatinib, or Erlotinib for 24 hours, then different concentrations of HuCTB006 were added to the combined group, the results were determined at 24 hours after the treatments.

TABLE 14
CDI of HuCTB006 and Targeted Drug Combinations on SUM102

| Concentration | CDI | | | | | |
|---|---|---|---|---|---|---|
| of HuCTB006 | Sorafenib | | Lapatinib | | Erlotinib | |
| (ng/mL) | 10 μM | 5 μM | 10 μM | 5 μM | 10 μM | 5 μM |
| 1000 | 1.26 | 1.04 | 1.40 | 1.33 | 1.18 | 1.30 |
| 500 | 1.09 | 1.05 | 1.59 | 1.58 | 1.31 | 1.58 |
| 250 | 0.93* | 1.03 | 1.39 | 1.48 | 1.31 | 1.52 |
| 125 | 1.15 | 0.98* | 1.47 | 1.42 | 1.33 | 1.39 |
| 62.5 | 1.21 | 1.00 | 1.38 | 1.25 | 1.28 | 1.29 |

*Synergy (CDI <1);
**Significant synergy (CDI <0.7).

4.4.3 DY36T2 Cell Line

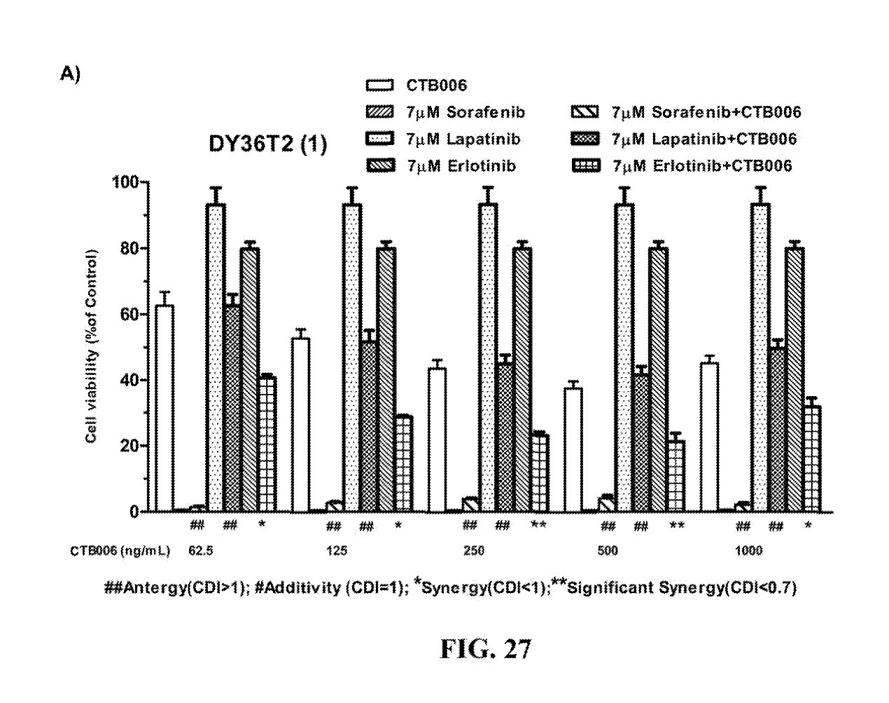

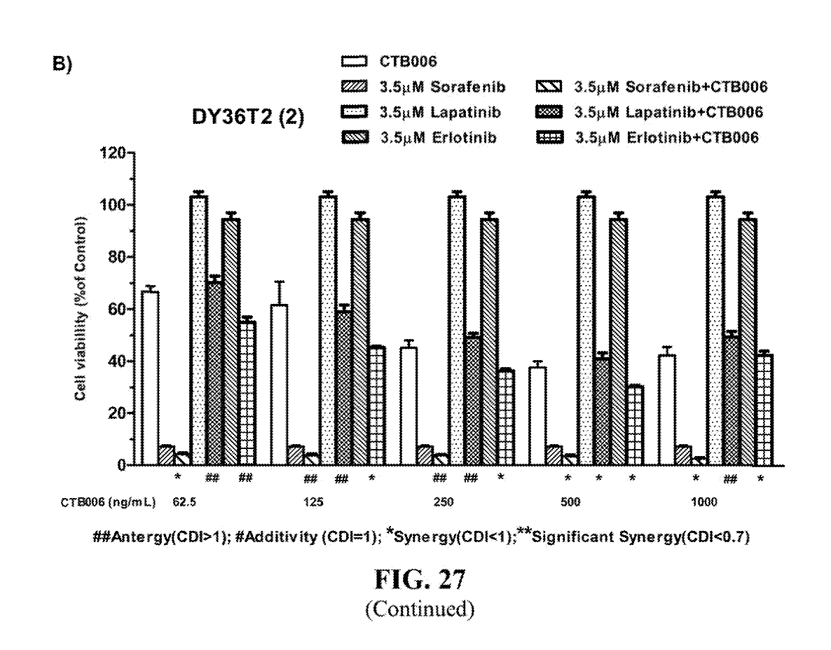

Breast cancer cell line DY36T2 cells were treated with the combination of different concentrations of HuCTB006, and 10 μM or 5 μM targeted drugs, including Sorafenib, Lapatinib and Erlotinib. The CDI values were shown in Table 15. The CDI values were much higher than 1 when DY36T2 was treated with the combination of different concentrations of HuCTB006 and 10 μM Sorafenib, indicating that the cytotoxic effect of HuCTB006 and 10 μM Sorafenib combinations on DY36T2 cells was significantly antergy as shown in FIG. 27. The cytotoxic effect of HuCTB006 and 5 μM Sorafenib combinations on DY36T2 cells was synergistic when the concentration of HuCTB006 was 1000, 125 and 62.5 ng/mL. The cytotoxic effect of HuCTB006 and Lapatinib combinations on DY36T2 cells was antergy except for the combination of 125 ng/mL HuCTB006 and 5 μM Lapatinib. Further, most of the CDI values were less than 1 when DY36T2 was treated with different concentrations of HuCTB006 and Erlotinib combinations, indicating that the cytotoxic effect of HuCTB006 and Erlotinib combinations on DY36T2 cells was synergistic. The cells were cultured at 37° C. and 5% of CO₂, and in culture medium of IMEM containing 10% of FBS. The cells were treated with Sorafenib, Lapatinib, or Erlotinib for 24 hours, then different concentrations of CTB006 were added to the combined group, the results were determined at 24 hours after the treatments.

TABLE 15
CDI of HuCTB006 and Targeted Drug Combinations on DY36T2

| Concentration | CDI | | | | | |
|---|---|---|---|---|---|---|
| of HuCTB006 | Sorafenib | | Lapatinib | | Erlotinib | |
| (ng/mL) | 10 μM | 5 μM | 10 μM | 5 μM | 10 μM | 5 μM |
| 1000 | 7.93 | 0.82* | 1.18 | 1.13 | 0.89* | 1.06 |
| 500 | 18.19 | 1.28 | 1.19 | 1.05 | 0.71* | 0.85* |
| 250 | 14.72 | 1.19 | 1.11 | 1.05 | 0.67** | 0.85* |
| 125 | 8.70 | 0.88* | 1.05 | 0.93* | 0.68** | 0.78* |
| 62.5 | 3.74 | 0.89* | 1.07 | 1.02 | 0.81* | 0.87* |

*Synergy (CDI <1);
**Significant synergy (CDI <0.7).

### Example 5. In Vivo Efficacy—Human Tumor Cells

The effectiveness of HuCTB006 was examined using four human tumor cell lines (i.e., two colon cancer, one lung cancer, and one pancreatic cancer) in a subcutaneous model in nude mice. The results showed that HuCTB006 inhibited the tumor growth of colon cancer cells, lung cancer cells, and pancreatic cancer cells even at low or micro doses, while HuCTB006 treatment alone also showed the same therapeutic effects as chemotherapy drugs. The results were reproducible.

5.1 HuCTB006's Therapy Effects in Human Tumor Cells Subcutaneous Model

5.1.1 Drugs

HuCTB006 (Lot No. H61042F-P01) was obtained from Beijing Cotimes Biotech Co., Ltd.; Irinotecan and Gemcitabine were obtained from Jiangsu Hengrui Medicine Co., Ltd.; Taxol was obtained from Beijing Union Pharmaceutical Factory. The drug power was first dissolved in NS to reach a concentration 5 mg/ml, and then filtrated through a 0.2 μm filter membrane (PALL, Aodisc® Syringe Filter).

5.1.2 Cells and Culture

H2122, Colo205 and MIA-PaCa-2 cell lines were purchased from ATCC. Cells were cultured in an incubator at 37° C. with 5% CO₂. H2122 and Colo205 cells were cultured in 1640 culture media; MIA-PaCa-2 cells were cultured in DMEM; and WiDr cells were cultured in IMEM. All of the culture media contains 10% FCS.

5.1.3 Animals

The animals were four (4) to six (6) weeks old nude mice, purchased from experimental animal center, military medical science academy (Animal Certificate No.: SCXK-( [symbol] )007-004), or Vital River Laboratories (Animal Certificate No.: SCXK( [symbol] )2006-0009).

5.1.4 Instruments

IVC was purchased from Suzhou Fengshi Laboratory Animal Equipment Co., Ltd.; Vernier calipers were purchased from Beijing Yazhongboke Co. Ltd.; Electronic balance were Sartorius® (BT25S, MAX 21 g, d=0.01 mg) and Denver®.

5.1.5 Animal Experiments

Tumor cells (i.e., MIA-PaCa-2, WiDr, H2122, colo205) were inoculated in nude mice's flank at 5×10⁶ cells per mouse. The mice were grouped when the average volume of tumors reached about 100˜200 mm³. Dose schedules were carried out as shown in Table 16. “QW”=weekly dose. “Q3d”=dosed every third day. “BiW”=biweekly.

TABLE 16
Dose Schedules of the Animal Experiments

| | | | | | Administration |
|---|---|---|---|---|---|
| Tumor Cell | Groups | Drugs | Dose | | frequency |
| MIA-PaCa-2 | Control | NS | 100 | μl/mice | QW × 6W |
| | CTB006 high dose group | HuCTB006 | 10 | mg/kg | |
| | CTB006 middle dose group | | 1 | mg/kg | |
| | CTB006 low dose group | | 0.6 | mg/kg | |
| | CTB006 micro dose group | | 0.2 | mg/kg | |
| | Gemcitabine group | Gemcitabine | 60 | mg/kg | Q3d × 6W |
| Colo205 | Control | NS | 100 | μl/mice | QW × 4W |
| | CTB006 high dose group | HuCTB006 | 5.4 | mg/kg | |
| | CTB006 middle dose group | | 1.8 | mg/kg | |
| | CTB006 low dose group | | 0.6 | mg/kg | |
| | CTB006 micro dose group | | 0.2 | mg/kg | |
| | Irinotecan group | Irinotecan | 50 | mg/kg | BiW × 6 times |
| WiDr | control | NS | 100 | μl/mice | QW × 4W |
| | CTB006 high dose group | HuCTB006 | 2 | mg/kg | |
| | CTB006 middle dose group | | 0.67 | mg/kg | |
| | CTB006 low dose group | | 0.22 | mg/kg | |
| | CTB006 micro dose group | | 0.07 | mg/kg | |
| | Irinotecan group | Irinotecan | 50 | mg/kg | BiW×6 times |
| H2122 | control | NS | 100 | μl/mice | QW × 6W |
| | CTB006 high dose group | HuCTB006 | 5.4 | mg/kg | |
| | CTB006 middle dose group | | 1.8 | mg/kg | |
| | CTB006 low dose group | | 0.6 | mg/kg | |
| | CTB006 micro dose group | | 0.2 | mg/kg | |
| | Taxol group | Taxol | 24 | mg/kg | Q6d × 6W |

NS (i.e., Normal Saline), CTB006 and Irinotecan were administered intravenously (i.e., i.v.); Taxol and Gemcitabine were administered intraperitoneally (i.e., i.p.).

The bodyweight and major/minor axis of the mice were measured twice a week, and the tumor volumes were calculated using the formula: Tumor volume=1/2×major axis×minor axis. Tumor Growth Inhibition (TGI) was calculated using the formula: TGI (%)=(1−T/C)×100. “T” represents average tumor volume of the experimental group (cm³), and “C” represents average tumor volume of the control group (cm³). The animals were monitored at all times. The bodyweight and major/minor axis or mice were measured twice a week. When the experiments were concluded, mice were sacrificed and tumors were extracted and weighted.

5.1.6 Statistical Method

Tumor volumes were represented with average±standard error. Data analysis was done using Spss16.0, One-Way ANOVA, LSD (homogeneity of variance) or Dunnett T3 (non-homogeneity of variance). Data transformation uses log, and the confidence interval was 95%. P<0.05 represents significant difference.

5.2 HuCTB006's Therapeutic Effects on Human Pancreatic Cancer Tumor Cells MIA-PaCa-2 Subcutaneous Model

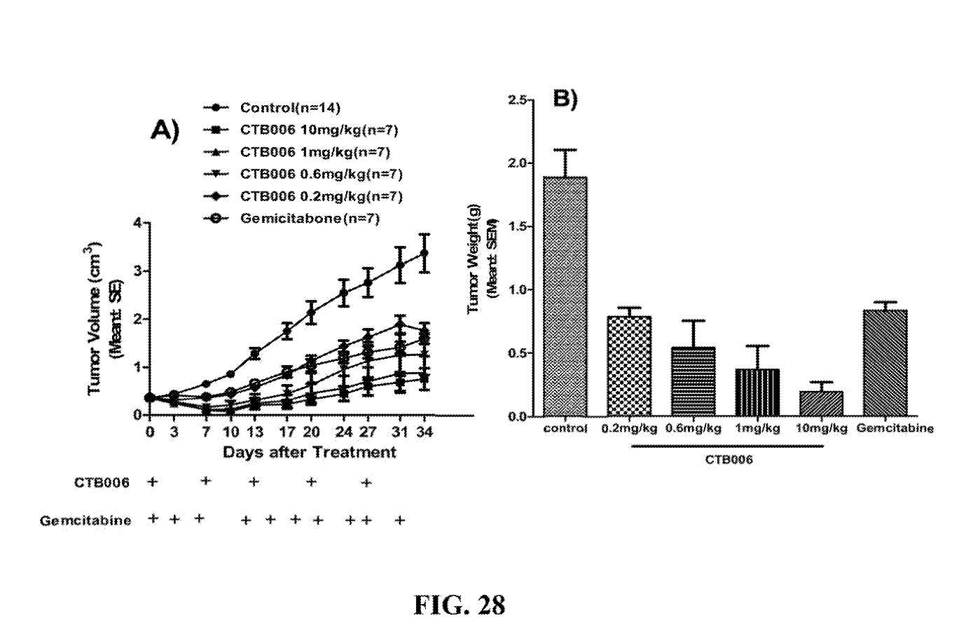

As shown in FIG. 28, HuCTB006 had an effect on the tumor growth in the MIA-PaCa-2 subcutaneous model. During the 34 days of treatment, tumor volumes were measured and calculated. In contrast to the control group, tumor volumes of all the experimental groups decreased notably and significantly, as showed in Table 17. Tumor growth inhibitions (TGIs), when calculated using tumor volume for the four HuCTB006 dose groups (i.e., 10 mg/kg, 1 mg/kg, 0.6 mg/kg, and 0.2 mg/kg) and gemcitabine (i.e., 60 mg/kg) group, were 77.49%, 73.86%, 62.38%, 47.58% and 52.60%, respectively. TGIs, when calculated using the tumor weight, were 89.71%, 80.32%, 71.09%, 58.15% and 55.66%, respectively. TGIs, based on either tumor volume or tumor weight, showed a dose-dependent response of the HuCTB006 treatments. The higher the dosage of HuCTB006, the more tumor growth inhibition. Unexpected and surprisingly, some animals of HuCTB006 groups showed tumor regression, which was not found in the chemotherapy group (e.g., gemcitabine).

TABLE 17
The Effect of HuCTB006 on Tumor Growth in MIA-Paca-2 Subcutaneous Model

| | Time of tumor | | T/C % | TGI | | TGI |
|---|---|---|---|---|---|---|
| | volume | Tumor | (Calculated | (Calculated | Tumor | (Calculated |
| | changed | Volume (cm³) | using tumor | using tumor | weight (g) | using tumor |
| Groups | significantly¹ | Mean ± SEM | volumes) | volumes) | Mean ± SEM | weight) |
| Control | 7 | 3.37 ± 0.40 | — | — | 1.89 ± 0.22 | — |
| HuCTB006 | 7² | 0.76 ± 0.22 | 23.01% | 77.49%** | 0.19 ± 0.08 | 89.71%** |
| 10 mg/kg | | | | | | |
| HuCTB006 | — | 0.88 ± 0.35 | 24.02% | 73.86%** | 0.37 ± 0.19 | 80.32%**³ |
| 1 mg/kg | | | | | | |
| HuCTB006 | — | 1.27 ± 0.43 | 38.43% | 62.38%** | 0.54 ± 0.21 | 71.09%**⁴ |
| 0.6 mg/kg | | | | | | |
| HuCTB006 | 13 | 1.76 ± 0.15 | 52.01% | 47.58%*⁵ | 0.79 ± 0.07 | 58.15%* |
| 0.2 mg/kg | | | | | | |
| Gemcitabine | 10 | 1.60 ± 0.11 | 47.40% | 52.60%** | 0.84 ± 0.07 | 55.66%* |

The significance (i.e., *P < 0.05) in the table were determined by comparing the treatment groups to the control group. In the treatment groups, the higher number of * represents more significant difference (¹Time of tumor volume changed significantly was represented by days after first treatment; ²the tumor volume was decreased significantly on Day 7 after first treatment; and since Day 13, there had been no significant difference when compared with the control group; ³there were no significant difference among HuCTB006 1 mg/kg group. HuCTB006 0.2 mg/kg group and gemcitabinc group; ⁴there were no significant difference among HuCTB006 0.6 mg/kg group, HuCTB006 0.2 mg/kg group and gemcitabinc group; ⁵there was significant difference between HuCTB006 0.2 mg/kg group and HuCTB006 10 mg/kg group; but no significant difference when compared with other groups.)

In a repeated experiment, the results were similar. TGIs, when calculated based on the tumor volume of the four HuCTB006 dose groups and gemcitabine group, were 58.99%, 87.52%, 85.35%, 59.52% and 34.33%, respectively. TGIs, when calculated based on the tumor weight, were 71.93%, 92.02%, 91.39%, 72.38% and 31.30%, respectively. TGIs, based on either tumor volume or weight, showed a dose-dependent response of HuCTB006 treatments. Unexpected and surprisingly, some animals of HuCTB006 groups also showed tumor regression, which was not found in the chemotherapy group alone (e.g., gemcitabine).

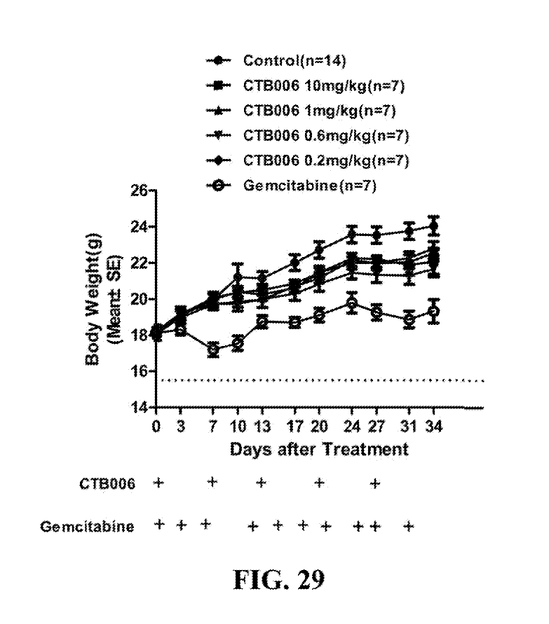

As shown in FIG. 29, the bodyweights of the control group and the HuCTB006 treatment groups showed slow increase over the time, indicating that the toxicity of HuCTB006 is low. In contrast, the bodyweights of mice in the gemcitabine treatment group decreased significantly by the third dosing, which recovered after one time drug withdrawal. Bodyweights were maintained at acceptable level thereafter. The results were similar in a repeated experiment.

5.3 HuCTB006's Therapy Effects in Human Colon Cancer Tumor Cells Colo205 Subcutaneous Model

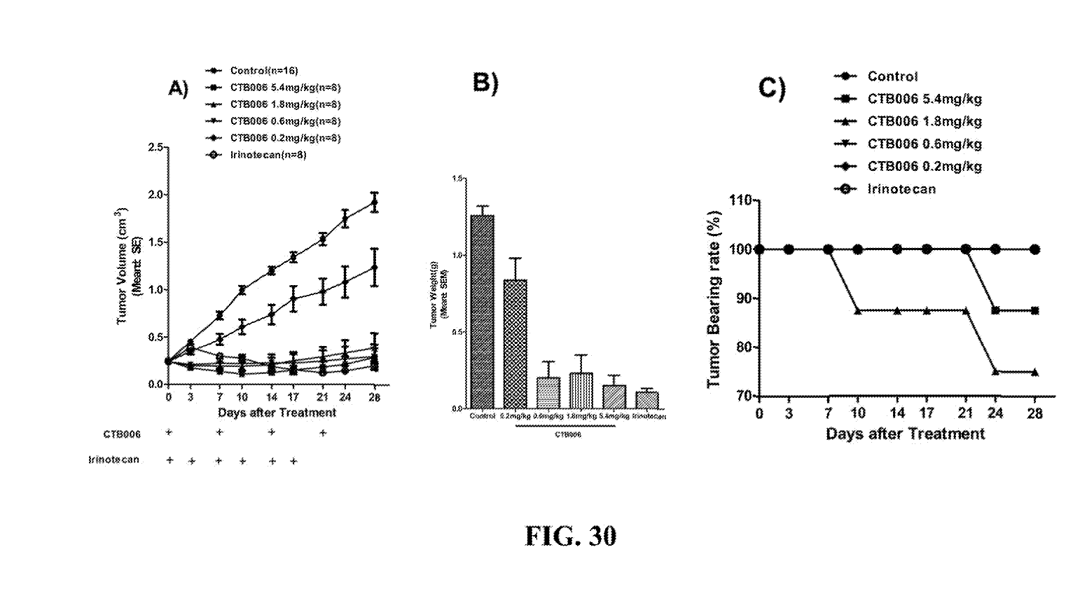

As shown in FIG. 30, HuCTB006 treatment had an effect on human colon cancer tumor cell line Colo25. Results were calculated using the tumor volumes on Day 28 after treatment. Tumor volumes of all of the experimental groups, excepted HuCTB006 0.2 mg/kg group, decreased notably when compared to the control group, as showed in Table 18. TGIs, when calculated using tumor volume of four different doses of CTB006 treatment groups and the Irinotecan group, were 85.24%, 80.07%, 84.78%, 35.62% and 89.81%, respectively; TGIs, when calculated using tumor weight, were 88.15%, 81.78%, 84.07%, 33.30% and 91.54%, respectively; showing a dose-dependent response of the HuCTB006 treatments. In this experiment, surprisingly and unexpectedly, four animals of HuCTB006 treatment groups showed tumor regression, while none was found in the Irinotecan group.

TABLE 18
The Effect of HuCTB006 Treatment on Tumor Growth in Colo205 Subcutaneous Model

| | Time of tumor | | T/C % | TGI | | TGI |
|---|---|---|---|---|---|---|
| | volume | Tumor | (Calculated | (Calculated | Tumor | (Calculated |
| | changed | Volume (cm³) | using tumor | using tumor | weight (g) | using tumor |
| Groups | significantly¹ | Mean ± SEM | volume) | volume) | Mean ± SEM | weight) |
| Control | 3 | 1.92 ± 0.10 | — | — | 1.26 ± 0.06 | — |
| HuCTB006 | 7~10² | 0.28 ± 0.10 | 14.80% | 85.24%* | 0.15 ± 0.07 | 88.15%* |
| 5.4 mg/kg | | | | | | |
| HuCTB006 | — | 0.38 ± 0.16 | 19.91% | 80.07%* | 0.23 ± 0.12 | 81.78%* |
| 1.8 mg/kg | | | | | | |
| HuCTB006 | — | 0.29 ± 0.13 | 14.41% | 84.78%* | 0.20 ± 0.10 | 84.07%* |
| 0.6 mg/kg | | | | | | |
| HuCTB006 | 10 | 1.24 ± 0.20 | 66.90% | 35.62% | 0.84 ± 0.14 | 33.30% |
| 0.2 mg/kg | | | | | | |
| Irinotecan | 3 & 17³ | 0.20 ± 0.04 | 10.06% | 89.81%* | 0.11 ± 0.02 | 91.54%* |

The significance (i.e., *P < 0.05) in the table were determined by comparing the treatment groups to the control group (¹Days after first treatment. ²Tumor volume was decreased significantly between Day 7 and Day 10, and there had been no significant difference compared with control group later. ³Tumor volume was increased significantly on the 3rd day after first treatment, but no significant difference between Day 7 and Day 10; and since Day 17. the tumor volume was decreased significantly, and there was no significant difference on Day 28).

In a repeated experiment, similar results were obtained. TGIs, when calculated using the tumor volume of the four dosing HuCTB006 treatment groups and then Irinotecan group, were 87.12%, 93.65%, 79.75%, 56.16% and 91.64%, respectively; TGIs, when calculated using the tumor weights, were 89.90%, 94.33%, 82.34%, 58.68% and 93.76%, respectively; showing a dose-dependent response of the HuCTB006 treatments. In the experiment, tumor regression was not observed in any groups.

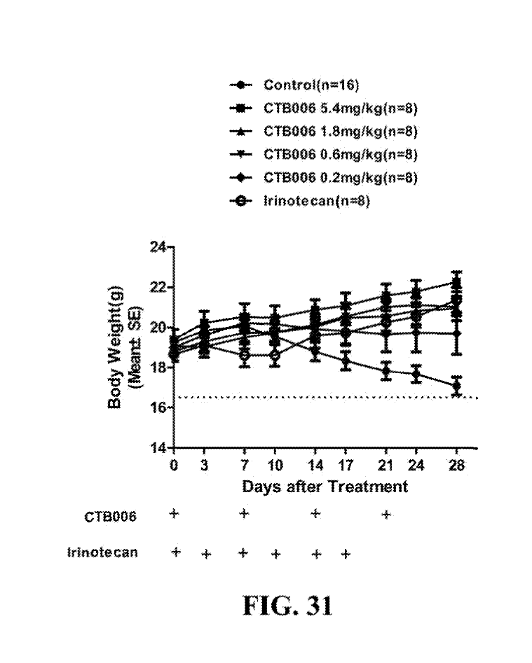

As shown in FIG. 31, since Day 10, the bodyweights of control group had slowly decreased, but all of the HuCTB006 treatment groups did not show any notably changes. The decrease of bodyweights of the control group is likely related to fast tumor growth. The results were similar in a repeated experiment.

5.3 CTB006's Therapy Effects in Human Colon Cancer Tumor Cells Widr Subcutaneous Model

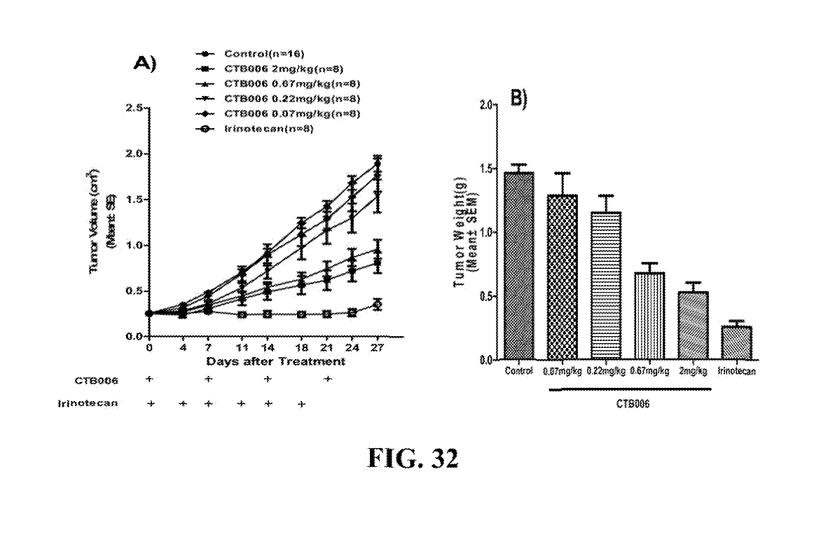

As shown in FIG. 32, HuCTB006 treatments had an effect on tumor growth on Widr cells in a subcutaneous model. Results were calculated using the tumor volumes at Day 27 after treatment. Compared to the tumor volumes of the control group, the tumor volumes of all the experimental groups decreased notably, excepted for the HuCTB006 0.07 mg/kg group, as showed in Table 19. Tumor growth inhibitions (TGIs) of the four HuCTB006 dose groups and the Irinotecan group, when calculated using tumor volume, were 57.34%, 49.42%, 18.71%, 6.61% and 81.34%, respectively; TGIs, when calculated using tumor weight, were 63.81%, 53.48%, 21.13%, 11.99% and 82.33%, respectively; showing a dose-dependent response of the CTB006 treatments.

In a repeated experiment, similar results were obtained. TGIs, when calculated using tumor volume of the four different doses of HuCTB006 groups and the Irinotecan group, were 53.77%, 53.73%, 54.98%, 36.21% and 71.13%, respectively; TGIs, when calculated using tumor weight, were 53.75%, 49.85%, 57.56%, 39.94% and 72.30%, respectively; showed a dose-dependent response of the CTB006 treatments as shown in Table 19.

TABLE 19
The Effect of HuCTB006 treatment on the Tumors in Widr Subcutaneous Model

| | Time of | | | | | |
|---|---|---|---|---|---|---|
| | tumor | | T/C % | TGI | | TGI |
| | volume | Tumor | (Calculated | (Calculated | Tumor | (Calculated |
| | changed | Volume (cm³ | using tumor | using tumor | weight (g) | using tumor |
| Groups | significantly | Mean ± SEM | volume) | volume) | Mean ± SEM | weight) |
| Control | 4 | 1.89 ± 0.09 | — | — | 1.46 ± 0.06 | — |
| HUCTB006 | 14 | 0.80 ± 0.11 | 42.17% | 57.34% ^{abc} | 0.53 ± 0.08 | 63.81% ^{a} |
| 2 mg/kg | | | | | | |
| HUCTB006 | 11 | 0.96 ± 0.10 | 49.54% | 49.42% ^{abc} | 0.68 ± 0.08 | 53.48% ^{a} |
| 0.67 mg/kg | | | | | | |
| HUCTB006 | 7 | 1.54 ± 0.18 | 81.49% | 18.71% ^{ac} | 1.16 ± 0.13 | 21.13% |
| 0.22 mg/kg | | | | | | |
| HUCTB006 | 7 | 1.76 ± 0.18 | 90.16% | 6.61% ¹ | 1.29 ± 0.18 | 11.99% |
| 0.07 mg/kg | | | | | | |
| Irinotecan | 27 | 0.35 ± 0.06 | 18.55% | 81.34% ^{a} | 0.26 ± 0.04 | 82.33% ^{ab} |

The significance (i.e., *P < 0.05) in the table were determined by comparing the treatment groups to the control group. There were no significant difference when the No. of letters were the same (¹ There was no significant difference between the HuCTB006 0.22 mg/kg group and the HuCTB006 0.07 mg/kg group, P = 0.224).

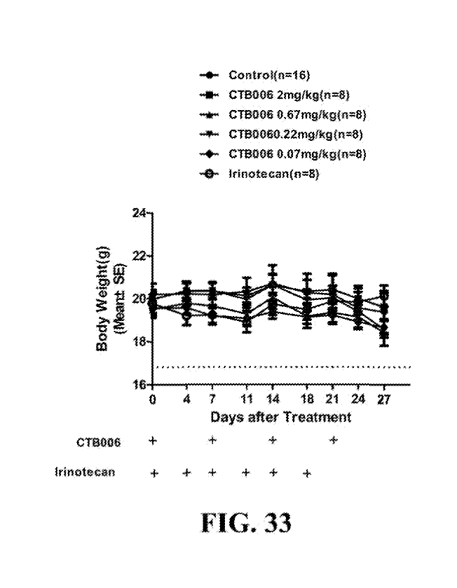

As shown in FIG. 33, in the experiment, the bodyweights of all the groups did not show any notable changes. The results were similar in a repeated experiment.

5.4 HuCTB006's Therapy Effects in Human Lung Cancer Tumor Cells H2122 Subcutaneous Model

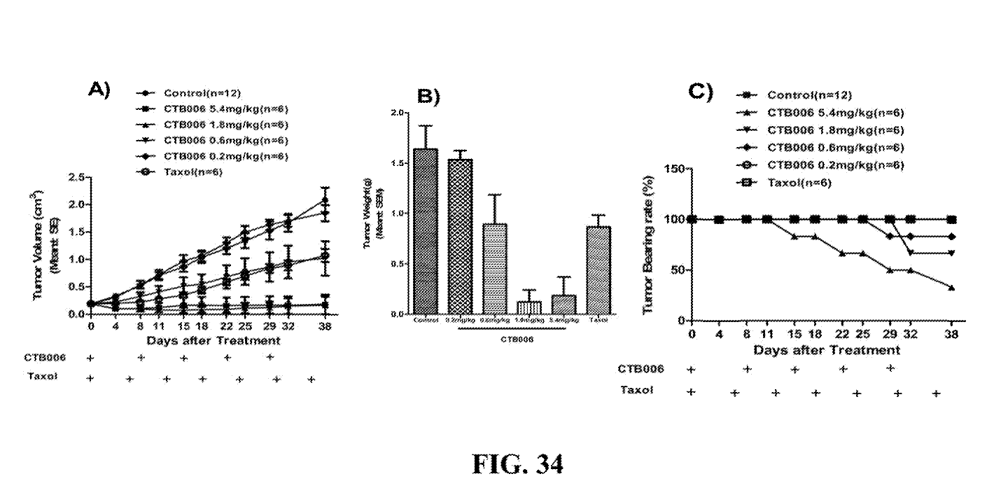

As shown in FIG. 34, HuCTB006 treatment had an effect on tumor growth of H2122 cells in a subcutaneous model. Results were calculated using the tumor volume at Day 38 days after treatment. Compared to the tumor volumes of the control group, excepted HuCTB006 0.2 mg/kg group and CTB006 0.6 mg/kg group, the tumor volumes in all of the experimental groups decreased notably, as shown in Table 20. Tumor growth inhibitions (TGIs) of four different HuCTB006 dose groups and the Taxol group, when calculated using tumor volume, were 91.23%, 92.08%, 51.06%, 11.56% and 48.38%, respectively; TGIs, when calculated using tumor weight, were 8.62%, 92.48%, 45.66%, 6.45% and 47.18%, respectively, showing a dose-dependent response of the HuCTB006 treatments. In this experiment, unexpectedly and surprisingly, some animals of HuCTB006 treatment groups showed tumor regression, while none was observed in the chemotherapy Taxol group.

In a repeated experiment, similar results were obtained. Tumor growth inhibitions (TGIs) of four different HuCTB006 dose groups and Taxol group, when calculated using tumor volume, were 91.69%, 60.12%, 67.66%, 35.26% and 54.28%, respectively; TGIs, when calculated using tumor weight, were 92.25%, 64.21%, 65.94%, 32.46% and 50.44%, respectively; showing a dose-dependent response of the HuCTB006 treatments. In this experiment, unexpectedly and surprisingly some animals of CTB006 treatment groups showed tumor regression, while none was observed in the chemotherapy Taxol group.

TABLE 20
The Effect of HuCTB006 Treatment on the Tumors in H2122 Subcutaneous Model

| | | | T/C % | TGI | | TGI |
|---|---|---|---|---|---|---|
| | Time of tumor | Tumor | (Calculated | (Calculated | Tumor | (Calculated |
| | volume changed | Volume (cm³) | using tumor | using tumor | weight (g) | using tumor |
| Groups | significantly | Mean ± SEM | volumes) | volumes) | Mean ± SEM | weight) |
| Control | 4 | 2.09 ± 0.23 | — | — | 1.64 ± 0.23 | — |
| HUCTB006 | | | | | | |
| 5.4 mg/kg | — | 0.18 ± 0.18 | 5.54% | 91.23%** | 0.19 ± 0.18 | 88.62%*¹ |
| HUCTB006 | | | | | | |
| 1.8 mg/kg | — | 0.16 ± 0.16 | 6.46% | 92.08%**² | 0.12 ± 0.12 | 92.48%* |
| HUCTB006 | | | | | | |
| 0.6 mg/kg | — | 1.02 ± 0.31 | 50.75% | 51.06%³ | 0.89 ± 0.29 | 45.66%⁴ |
| HUCTB006 | | | | | | |
| 0.2 mg/kg | 8 | 1.85 ± 0.15 | 92.13% | 11.56% | 1.54 ± 0.09 | 6.45%⁵ |
| Taxol | 11 | 1.08 ± 0.12 | 55.33% | 48.38% * | 0.87 ± 0.12 | 47.18% |

The significance (i.e., *P < 0.05) in the table were determined by comparing the treatment groups to the control group. There were no significant difference when the No. of letters were the same (¹There was no significant difference between the HuCTB006 5.4 mg/kg group and the HuCTB006 0.6 mg/kg group; and there was also no significant difference between the HuCTB006 5.4 mg/kg group and the Taxol group. ²There was significant difference between the HuCTB006 1.8 mg/kg group and the Taxol group. ³There was no significant difference between the HuCTB006 0.6 mg/kg group and the other groups. ⁴There was no significant difference between the HuCTB006 1.8 mg/kg group and the HuCTB006 0.6 mg/kg group. ⁵There was significant difference between the HuCTB006 0.2 mg/kg group and the Taxol group).

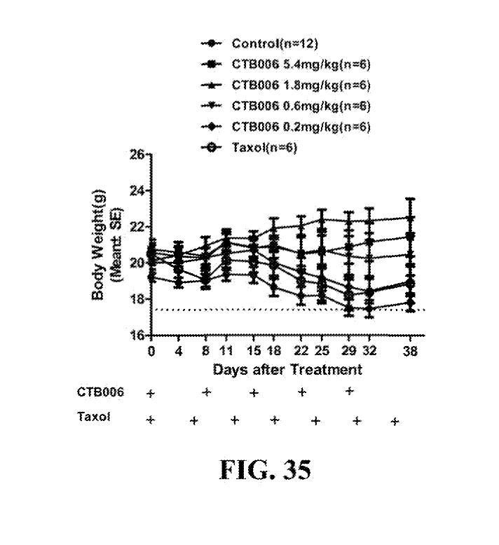

As shown in FIG. 35, the bodyweights of the Taxol group, HuCTB006 0.2 mg/kg group and control group decreased, but were maintained at an acceptable level. The results were similar in a repeated experiment.

### Example 6. In Vivo Efficacy—Human Tumor Tissues

The effectiveness of HuCTB006 treatments was examined on five human tumor tissues in a subcutaneous models in nude mice, the results showed synergistic or additive effects of the CTB006 treatment in combination with chemotherapy drugs, and the therapeutic effects of CTB006 were better than chemotherapy drugs alone.

6.1 HuCTB006's Therapy Effects in Patients Primary Tumor Tissue Subcutaneous Model

6.1.1 Drugs

HuCTB006 was obtained from Beijing Cotimes Biotech Co., Ltd./Shenzhen Lonnryonn Pharmaceutical Co., Ltd.; Taxol was obtained from Beijing SL Pharmaceutical Co., Ltd./Beijing Union Pharmaceutical Factory; Carboplatin was obtained from Qilu Pharmaceutical Co., Ltd.; INF-α-2b was obtained from Schering-Plough (Brinny) Co., Ltd.; Irinotecan was obtained from Jiangsu Hengrui Medicine Co., Ltd.

6.1.2 Tissue

Colon cancer tissue CS146 was obtained from The Affiliated Tumor Hospital of China Academy of Medical Science; Colon cancer tissues CS182 and CS263, and lung cancer tissues CS113 and CS225 were obtained from Chao-Yang Hospital.

6.1.3 Animals

The animals were four (4) to six (6) weeks old nude mice, purchased from experimental animal center, military medical science academy (Animal Certificate No.: SCXK-( [symbol] )007-004), or Vital River Laboratories (Animal Certificate No.: SCXK( [symbol] )2006-0009). NOD/SCID mice were implanted at four (4) to five (5) weeks old, which were purchased from Beijing HFK Bioscience Co. Ltd. (Animal Certificate No.: SCXK( [symbol] )2009-0004) or Vital River Laboratories (Animal Certificate No.: SCXK( [symbol] )2012-0001).

6.1.4 Instruments

The animal experiment related instruments were the same as 5.1.4. In addition, Dewaterer (Tissue-Tek, VIPTM5Jr) and Embedding machine (TECTM) were purchased from Japan Sakura; Chipper (Leica) was purchased from Leica Microsystems Limited; Paraffin expanding machine and Baking machine were purchased from Tianjin Tianli Aviation Electro-Mechanical Co. Ltd.; electrothermal constant-temperature dry box was purchased from Shanghai Yiheng Technology Co. Ltd.; and microscope was purchased from OLYPUS (Japan).

6.1.5 Animal Experiments

Fresh primary tumor tissues from patients were implanted in NOD/SCID mice's flank, which developed into a primary tumor, which was then transferred to the nude mice's flank to allow the tumor reaching an exponential growth phase, and the tumor was named 1# tumor, and so on. When the tumor volume and number reached passage requirements, they were implanted into the right flank of nude mice. When the tumors' average volumes reached 100-200 mm³, the mice were grouped. Dose schedules were carried out as shown in Table 21.

TABLE 21
Dose Schedules of the Animal Experiments (HuCTB006 is indicated as CTB006 in this table)

| Cancers | Groups | Drugs | Dose | | Administration frequency |
|---|---|---|---|---|---|
| CS146 | Control | NS | 100 | μl/mice | QW × 6w |
| (Colon, 2#) | CTB006 group | CTB006 | 10 | mg/kg | |
| | 5-Fu group | 5-Fu | 50 | mg/kg | |
| | CTB006 + 5-FU group | CTB006, | The same as the single dose group | | |
| | | 5-Fu | | | |
| CS182 | Control | NS | 100 | μl/mice | QW × 3w |
| (Colon, 5#) | CTB006 group | CTB006 | 10 | mg/kg | |
| | Irinotecan group | Irinotecan | 50 | mg/kg | BiW × 6 times |
| | CTB006 + Irinotecan group | CTB006, Irinotecan | The same as the single dose group | | |
| CS263 | Control | NS | 100 | μl/mice | QW × 4w |
| (Colon, 1#) | CTB006 group | CTB006 | 10 | mg/kg | |
| | Irinotecan group | Irinotecan | 50 | mg/kg | BiW × 6 times |
| | CTB006 + Irinotecan group | CTB006, Irinotecan | The same as the single dose group | | |
| CS113 | Control | NS | 100 | μl/mice | QW × 5w |
| (lung, 3#) | CTB006 group | CTB006 | 10 | mg/kg | |
| | Taxol + Carboplatin group | Taxol, | Taxol: 24 | mg/kg | Taxol: QW × 4w |
| | | carboplatin | Carboplatin: 30 | mg/kg | Carboplatin: QW × 2 times |
| | CTB006 + Taxol + Carboplatin | CTB006, taxol, | The same as the single dose group | | |
| | group | carboplatin | | | |
| CS225 | Control | NS | 100 | μl/mice | QW × 5w |
| (lung, 1#) | CTB006 group | CTB006 | 10 | mg/kg | |
| | cisplatin group | cisplatin | 5 | mg/kg | QW × 2w |

| | | |
|---|---|---|
| CTB006 + cisplatin group | CTB006, cisplatin | The same as the single dose group |

HuCTB006 and Irinotecan were administered i.v. and 5-Fu, Taxol, Carboplatin and cisplatin were administered i.p. When dosing two drugs together, chemotherapy drugs were administered more than four hours before the antibody treatment.

The bodyweight and major/minor axis of mice were measured twice a week, and Tumor Growth Inhibition (TGI) and tumor volume were calculated same as in 5.1.5. In addition, to evaluate the synergetic effect of drug combination in tumor growth, Jin Zhengjun Q value judgment method was used with the follow formula: Q=E_{a+b}/(Eₐ+E_{b}−Eₐ×E_{b}). E_{a+b} is the inhibition ratio of drug combination, Eₐ and E_{b} are the inhibition ratio of each of drug treatment when it is administered alone. Q<0.85 shows an antagonist effect, 0.85≤Q<1.15 shows an additive effect, and Q≥1.15 shows a synergism effect. The animals were monitored, and when the experiments were concluded, mice were sacrificed and tumors were extracted and weighted. All the animal experiments confer to the regulations of animal's welfare and the management of experimental animal usage.

6.1.6 Statistical Method

Tumor volumes were represented with average±standard error. Data analysis was done using Spss16.0, One-Way ANOVA, LSD (homogeneity of variance) or Dunnett T3 (non-homogeneity of variance). Data transformation uses log, and the confidence interval was 95%. P<0.05 represents significant difference.

6.2 the Combination of HuCTB006 and 5-Fu's Therapy Effects in Patients Primary Tumor Tissue (Colon Cancer, CS146, 2#) Subcutaneous Model

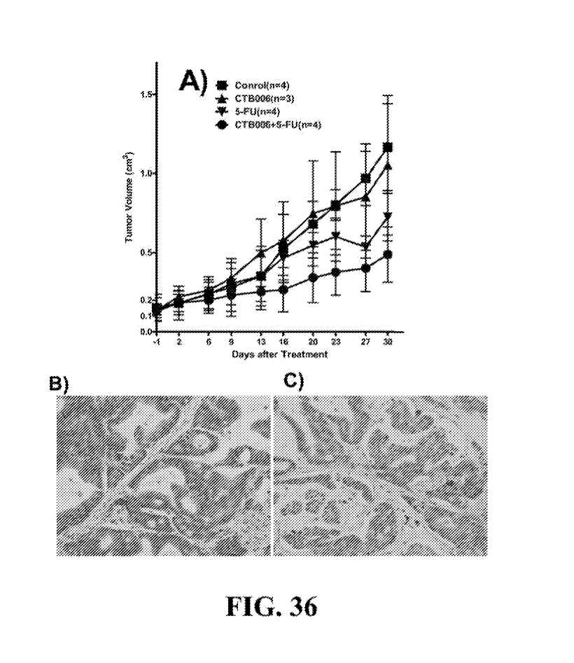

As shown in FIG. 36, the combination of HuCTB006 and 5-Fu had an effect on the tumors derived from a patient's primary tumor tissue (colon cancer, CS146, 2#) in a subcutaneous model. HuCTB006 did not, but 5-Fu did, have inhibitory effect on the growth of CS146 in the subcutaneous model. The results were calculated using the tumor volumes of Day 30 after treatment, and TGI was 37.95%. The combination of CTB006 and 5-FU had significant inhibition effect in the growth of CS146 (colon cancer) in the subcutaneous model. The results were calculated using the tumor weights of Day 30 after treatment, and TGI was 58.18%. The synergistic effect of drug combination was calculated using Jin Zhengjun Q value judgment method, which was Q=1.32, showing unexpected and surprising synergistic effect of the combination of HuCTB006 and 5-Fu treatment.

6.3 the Combination of HuCTB006 and Irinotecan's Therapy Effects in Patients Primary Tumor Tissue (Colon Cancer, CS182, 5#) Subcutaneous Model

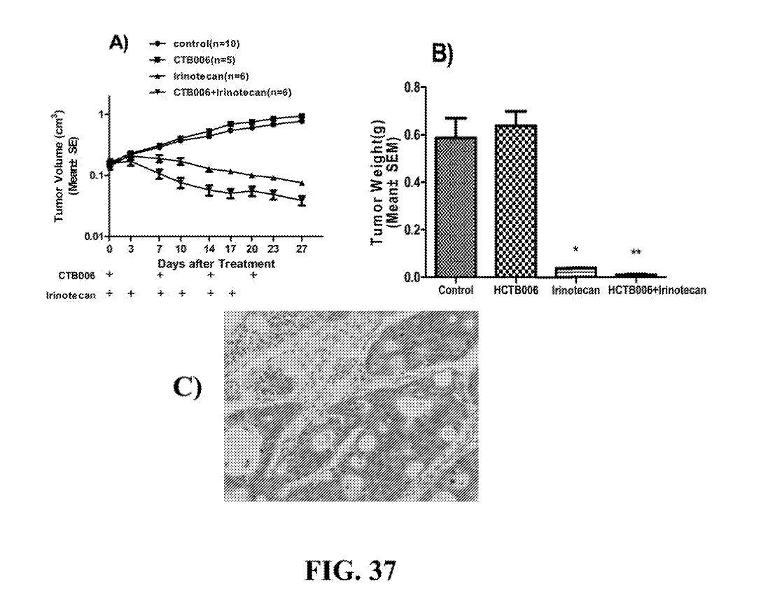

As shown in FIG. 37 and Table 22, the combination of HuCTB006 and irinotecan had effect on the tumors derived from a patient's primary tumor tissue (colon cancer, CS182, 5#) in a subcutaneous model. The results were calculated using the tumor volumes of Day 27 after treatment. Compared to the tumor volumes of the control group, the tumor volumes of the irinotecan group and the HuCTB006+ irinotecan group decreased notably, tumor growth inhibition (TGI) of HuCTB006, irinotecan, and CTB006+ irinotecan group were −21.15%, 90.36% and 95.00%, respectively. The synergistic effect of drug combination was calculated using Jin Zhengjun Q value judgment method, which was Q=1.07, showing an additive effect. TGIs, when calculated using tumor weight, were −9.22%, 93.46% and 98.29%, respectively. The synergetic effect of drug combination was calculated using Jin Zhengjun Q value judgment method, which was Q=1.06, also showing an additive effect.

TABLE 22
The Combination of HuCTB006 and Irinotecan's Effect on the Tumors in Patient's Primary
Tumor Tissue (Colon Cancer, CS182, 5#) in a Subcutaneous Model

| | Time of tumor | Tumor | T/C % | TGI | | TGI |
|---|---|---|---|---|---|---|
| | volume | Volume | (Calculated | (Calculated | Tumor | (Calculated |
| | changed | (cm³) | using tumor | using tumor | weight (g) | using tumor |
| Groups | significantly | Mean ± SEM | volumes) | volumes) | Mean ± SEM | weight) |
| Control | 3 | 0.78 ± 0.07 | — | — | 0.59 ± 0.08 | — |
| HUCTB006 | 3 | 0.95 ± 0.06 | 102.39% | −21.15% | 0.64 ± 0.06 | −9.22% |
| Irinotecan | 20 (↓)¹ | 0.08 ± 0.01 | 8.47% | 90.36%* | 0.04 ± 0.00 | 93.46%* |
| HuCTB006 + | 10 (↓) | 0.04 ± 0.01 | 4.30% | 95.00%** | 0..01 ± 0.00 | 98.29%** |
| Irinotecan | | | | | | |
| Q | — | — | — | 1.07 | — | 1.06 |

The significance (i.e., *P < 0.05) in the table were determined by comparing the treatment groups to the control group (¹The time of the decreasing of tumor volume when compared to pre-dosage).

6.4 the Combination of HuCTB006 and Irinotecan's Therapy Effects in Patient's Primary Tumor Tissue (Colon Cancer, CS263, 1#) in a Subcutaneous Model

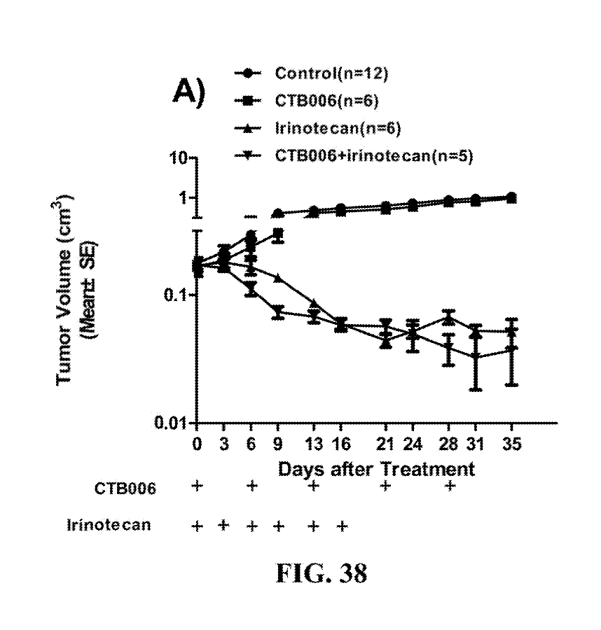

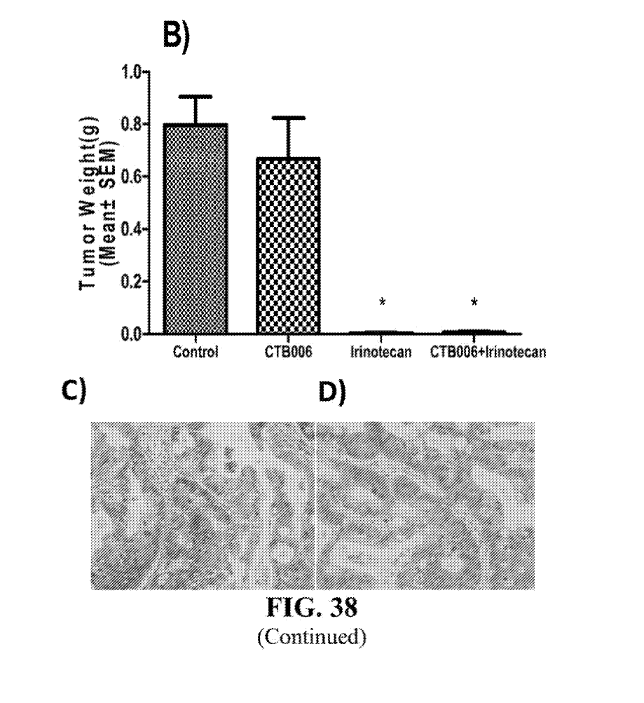

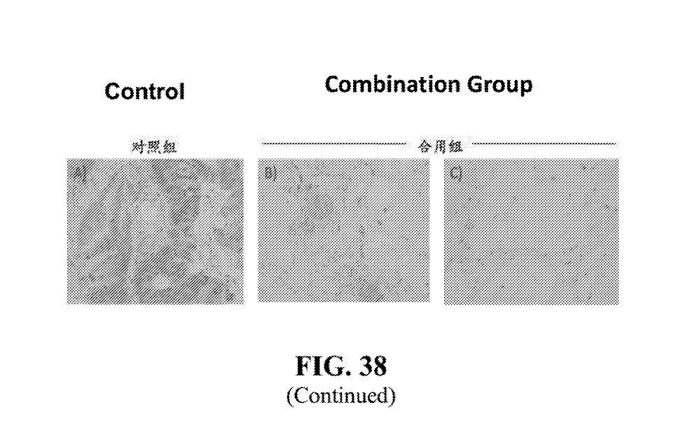

As shown in FIG. 38 and Table 23, the combination of HuCTB006 and irinotecan had an effect on the tumors derived from patient's primary tumor tissue (colon cancer, CS263, 1#) in a subcutaneous model. The results were calculated using the tumor volumes of Day 35 after treatment.

Compared to the tumor volumes of the control group, the tumor volumes of the irinotecan group and the HuCTB006+ irinotecan group decreased notably, tumor growth inhibitions (TGIs) of CTB006, irinotecan and HuCTB006+ irinotecan groups were 9.42%, 95.15% and 96.52%, respectively. The synergy of drug combination was calculated using Jin Zhengjun Q value judgment method, which was Q=1.01, showing an additive effect. TGIs, when calculated using tumor weight, were 16.11%, 99.58% and 99.25%, respectively. The synergetic effect of drug combination was calculated using Jin Zhengjun Q value judgment method, resulting Q=1.00, also showing an additive effect.

In this experiment, two animals in the HuCTB006+ irinotecan combination group showed tumor regression on Day 24 and Day 31. In the irinotecan group, one animal showed tumor regression on Day 35.

In a pathological examination, two out of three samples in the combination group (i.e., HuCTB006+irinotecan) did not show the existence of any tumor cells, and the goiter contained fibrous connective tissue as shown in FIG. 38. In the anatomy and pathological examination, there were four samples did not show any tumor cells, and there was at least one sample in the chemotherapy group had tumor cells, indicating that the therapeutic effectiveness of the combination group was much better than the chemotherapy group alone.

TABLE 23
The Combination Of HuCTB006 and Irinotecan's Effect on the Tumors in Patient's Primary
Tumor Tissue (Colon Cancer, CS263, 1#) in a Subcutaneous Model

| | | Tumor | T/C % | TGI | | TGI |
|---|---|---|---|---|---|---|
| | Time of tumor | Volume | (Calculated | (Calculated | Tumor | (Calculated |
| | volume changed | (cm³) | using tumor | using tumor | weight (g) | using tumor |
| Groups | significantly | Mean ± SEM | volumes) | volumes) | Mean ± SEM | weight) |
| Control | 6 | 1.06 ± 0.10 | — | — | 0.80 ± 0.11 | — |
| HUCTB006 | 13 | 0.96 ± 0.22 | 89.71% | 9.42% | 0.67 ± 0.16 | 16.11% |
| Irinotecan | 16 (↓)¹ | 0.05 ± 0.01 | 5.17% | 95.15%* | 0.003 ± 0.002 | 99.58%* |
| CTB006 + | 6 (↓) | 0.04 ± 0.02 | 3.67% | 96.52%* | 0..006 ± 0.004 | 99.25%* |
| Irinotecan | | | | | | |
| Q | — | — | — | 1.01 | — | 1.00 |

The significance (i.e., *P < 0.05) in the table were determined by comparing the treatment groups to the control group (¹The time of the decreasing of tumor volume when compared to pre-dosage).

6.5 the Combination of HuCTB006, Taxol and Carboplatin's Therapy Effects in Patients Primary Tumor Tissue (Lung Cancer, CS113, 3#) in a Subcutaneous Model

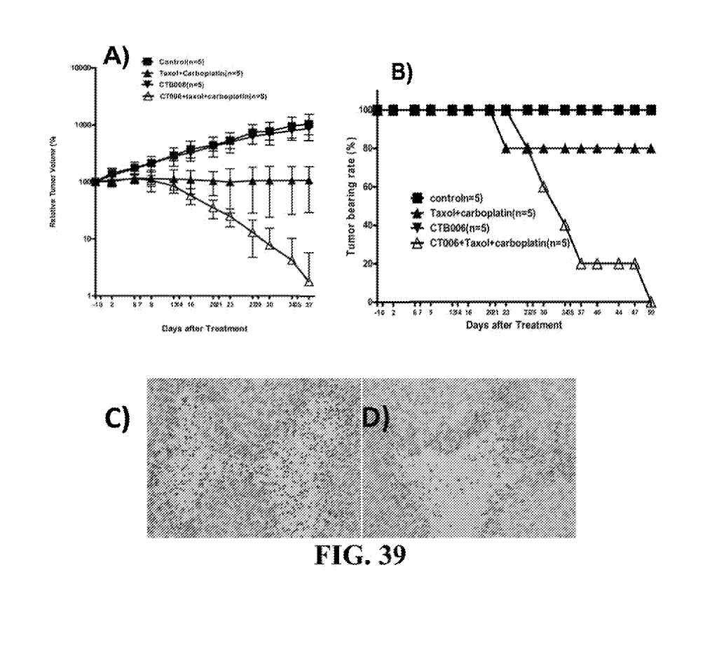

As shown in FIG. 39, the combination of HuCTB006, taxol and carboplatin had effect on the tumors derived from patient's primary tumor tissue (lung cancer, CS113, 3#) in a subcutaneous model.

The taxol+carboplatin group andHuCTB006+taxol+carboplatin group both had inhibitory effect on the growth of CS113 (i.e., lung cancer) in a subcutaneous model. TGIs of taxol+carboplatin group and HuCTB006+taxol+carboplatin group, when calculated using the tumor volumes of Day 23 after treatment, were 81.01% and 95.25%, respectively, showing that the effect was significant. The synergistic effect of drug combinations was calculated using Jin Zhengjun Q value judgment method, which was Q=1.16, showing a synergistic effect.

As shown in FIG. 39, on Day 37, 80% animals (4/5) of HuCTB006+taxol+carboplatin group showed tumor regression; on Day 50, all of the animals (5/5) showed tumor regression; while there was only one animal (1/5) showed tumor regression in taxol+carboplatin group. This also showed the therapy effectiveness of the combination group (i.e., HuCTB006+taxol+carboplatin) was unexpectedly and surprisingly better than the chemotherapy group alone.

6.6 the Combination of HuCTB006 and Cisplatin's Therapy Effects in Patient's Primary Tumor Tissue (Lung Cancer, CS225, 1#) in a Subcutaneous Model

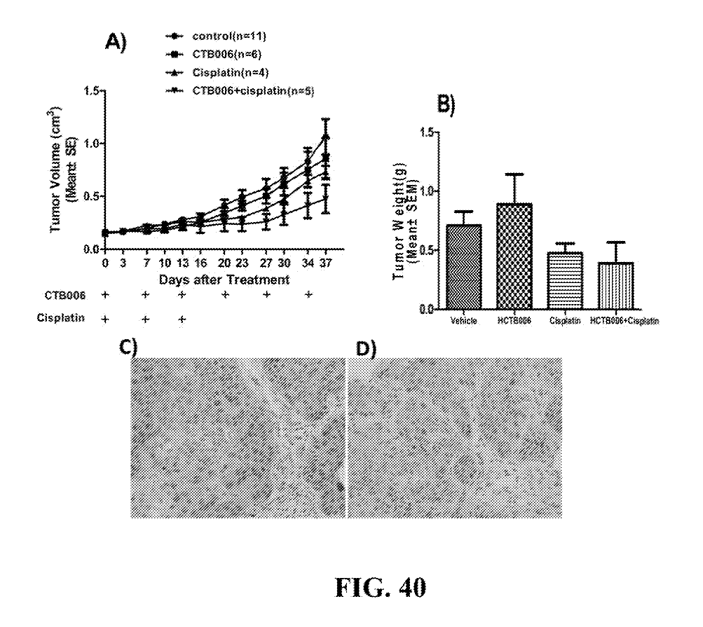

As shown in FIG. 40, the combination of HuCTB006 and cisplatin had an effect on the tumor derived from patient's primary tumor tissue (i.e., lung cancer, CS225, 1#) in a subcutaneous model.

TABLE 24
The Combination of HuCTB006 and Cisplatin Effect on the Tumors in Patients Primary Tumor
Tissue (Colon Cancer, CS225, 1#) in a Subcutaneous Model

| | | Tumor | T/C % | TGI | | TGI |
|---|---|---|---|---|---|---|
| | Time of tumor | Volume | (Calculated | (Calculated | Tumor | (Calculated |
| | volume changed | (cm³) | using tumor | using tumor | weight (g) | using tumor |
| Groups | significantly | Mean ± SEM | volumes) | volumes) | Mean ± SEM | weight) |
| Control | 10 | 1.06 ± 0.17 | — | | 0.71 ± 0.12 | — |
| HuCTB006 | 16 | 0.86 ± 0.19 | 79.86% | 19.65% | 0.89 ± 0.23 | −25.23% |
| Cisplatin | 7 | 0.73 ± 0.06 | 72.14% | 31.45% | 0.48 ± 0.08 | 32.66% |
| HuCTB006 + | —¹ | 0.48 ± 0.12 | 42.30% | 55.33%* | 0.39 ± 0.16 | 45.00% |
| Cisplatin | | | | | | |
| Q | — | — | — | 1.23 | — | 2.87 |

The significance (i.e., *P < 0.05) in the table were determined by comparing the treatment groups to the control group (¹The combination group did not show any significant change in all of the therapy).

The results were calculated using the tumor volumes on Day 37 after treatment. Compared to the tumor volumes of the control group, the tumor volumes of the HuCTB006+cisplatin group decreased notably. Tumor growth inhibitions (TGIs) of HuCTB006, cisplatin and HuCTB006+cisplatin group were 19.65%, 31.45% and 55.33%, respectively. The synergetic effect of drug combination was calculated using Jin Zhengjun Q value judgment method, which was Q=1.23, showing a synergistic effect. TGIs, when calculated using tumor weights, were −25.23%, 32.66% and 45.00%, respectively. The synergetic effect of drug combinations was calculated using Jin Zhengjun Q value judgment method, which was Q=2.87, also showing a synergistic effect.

### Example 7. Development of the Drs Quantitative Kit Accompanying the HuCTB006 Therapy

7.1 Reagents

Reagents used are anti-DR5 antibody, Clone: A10, Lot: 20110808, 1.2 mg/mL, prepared by Beijing Cotimes Biotech Ltd.; recombination DR5-rFC antigen, Lot: 20100415, 1 mg/mL, prepared by Beijing Cotimes Biotech Ltd.; HRP anti-DR5, Clone: 2B9, Lot: 20100825, prepared by Beijing Cotimes Biotech Ltd.; chemiluminescence substrate solution, Lot: 110546, purchased from KPL; BCA kit, Prod#: 23227, Lot: MF158389, purchased from Thermo Fisher; cell & tissue lysis solution, prepared by Beijing Cotimes Biotech Ltd.; other chemical reagents from Beijing Chemical Plant (China), all of analytical grade, and double-distilled water.

7.2 Instruments

Instruments used are chemiluminescence microplate reader, BHP9504, BeijingHamamatsu Photon Techniques Inc.; automated microplate washer, DEM-III, Beijing Tuopu Analytical Equipment Co. Ltd.; electric homoiothermic incubator, DHP-9162, Shanghai Yiheng Technology Co. Ltd.; vortex shaker, MS2, IKA; microplate shaker, MH-1, Haimen Qilinbeier Equipment Inc.; centrifuge, Microfuge16, Beckman coulter; 10 μL, 20 μL, 100 μL, 200 μL, 1000 μL micropipettors, Eppendorf.

7.3 Buffer Solution

Buffer solutions are coating solution: 0.02 mol/L phosphate buffer (pH=7.2); blocking solution: 0.02 mol/L phosphate buffer (PBS, pH=7.2, plus 1% BSA and 0.05% proclin-300); and washing solution: 0.02 mol/L PBS.

7.4 Determination of DR5 in Human Tumor Cell Line

There is a threshold (i.e., 0.2 ng/mL) for DR5 detection. The lower-than-threshold cell lines are not sensitive to HuCTB006, and part of the higher-than-threshold cell lines are sensitive to HuCTB006. Therefore, without being bound by theory, it can be considered that the cascade reactions of the downstream Caspase are activated only when DR5 expression reached a limited threshold, which then induce the apoptosis of the tumor cell lines. In other words, the high expression of DR5 can induce cell apoptosis pathway.

TABLE 25
The Relativity Between Concentration of DR5 in Human Tumor Cell
Lysis Solution and in vitro Cytotoxic Sensitivity of HuCTB006

| | | | In vitro |
|---|---|---|---|
| | | Conc. of DR5 | cytotoxic sensitivity |
| Category | Cell line | (ng/mL) | of CTB006 |
| B cell | Daudi | 0.163 | Non-responder |
| lymphoma | Raji | 0.053 | Non-responder |
| Lung cancer | H2122 | 0.805 | Responder |
| | SK-MES-1 | 0.258 | Responder |
| | H460 | 0.613 | Partial-responder |
| | A427 | 0.139 | Non-responder |
| | A549 | 0.473 | Non-responder |
| Liver cancer | SK-Hep-1 | 0.668 | Partial-responder |
| | 7402 | 0.144 | Non-responder |
| | Huh-7 | 0.035 | Non-responder |
| Colorectal cancer | COLO205 | 0.441 | Responder |
| | HCT116 | 0.208 | Responder |
| | SW480 | 0.149 | Partial-responder |
| | HT-29 | 0.198 | Non-responder |
| | SW620 | 0.19 | Non-responder |
| Ovarian cancer | SKOV3 | 0.168 | Non-responder |
| | OVCA3 | 0.088 | Non-responder |
| Breast | 2-LMP | 0.431 | Responder |
| cancer | MDA-MB- | 0.473 | Responder |
| | 231 | | |
| | SUM102 | 0.109 | Partial-responder |
| | BT-20 | 0.051 | Non-responder |
| | BT474 | 0.053 | Non-responder |
| | HCC1954 | 0.056 | Non-responder |
| | MDA-MB- | 0.013 | Non-responder |
| | 361 | | |
| | MDA-MB- | 0.119 | Non-responder |
| | 453 | | |
| | MDA-MB- | 0.042 | Non-responder |
| | 468 | | |
| | ZR-75-30 | 0.037 | Non-responder |
| Stomach | BGC823 | 0.152 | Partial-responder |
| cancer | NUGC3 | 0.761 | Partial-responder |
| | 7901 | 0.268 | Non-responder |
| | BGC803 | 0.151 | Non-responder |
| | MKN28 | 0.197 | Non-responder |
| | N87 | 0.088 | Non-responder |
| Fibrosarcoma | HT1080 | 1.685 | Non-responder |
| Pancreatic | BXPC3 | 0.33 | Responder |
| cancer | MIA-PaCa-2 | 0.709 | Responder |
| | Panc2.03 | 0.237 | Partial-responder |
| | ASPC-1 | 3.29 | Non-responder |
| | Capan-1 | 0.786 | Non-responder |
| | PANC-1 | 0.214 | Non-responder |

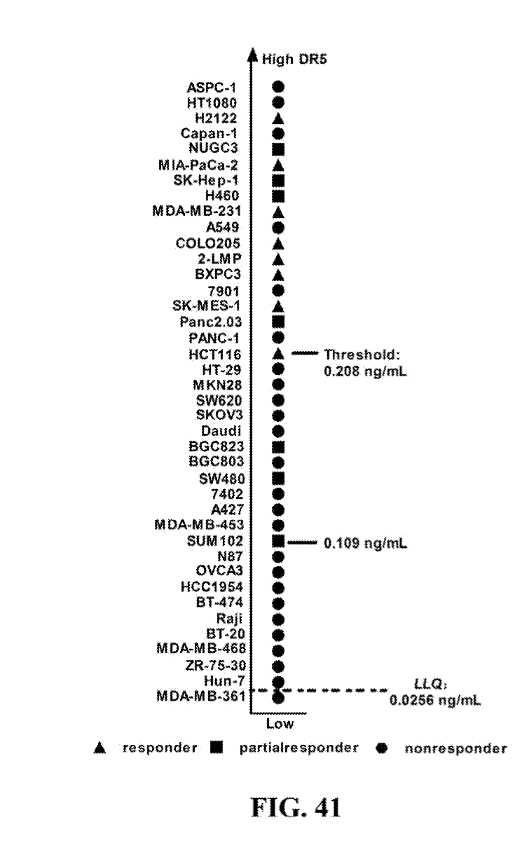

FIG. 41 shows the relativity between concentration of DR5 in human tumor cell lysis solution and in vitro cytotoxic sensitivity of HuCTB006.

7.5 Determination of DR5 in Clinical Patients Cancer Tissues

The cancer tissues and the relative adjacent tissues from clinical cancer patients were lysed in the lysis solution, and then the total protein concentration was determined by BCA kit. The total protein level in the lysis solution was then diluted to 8 mg/mL, and the DR5 level was determined using the CLEIA kit. The results were shown in Table 26.

TABLE 26
Comparison of DR5 Level Between Cancer Tissue and Adjacent Tissue

| | | Mean value | | | | |
|---|---|---|---|---|---|---|
| Cancer | Tissue | of DR5 | | | SE of the | |
| category | category | (ng/mL) | N | STD | mean | P |
| Colorectal | Cancer | 0.285 | 45 | 0.4346 | 0.0648 | 0.001 |
| cancer | tissue | | | | | |
| | Adjacent | 0.059 | 45 | 0.0301 | 0.0045 | |
| | Tissue | | | | | |
| Stomach | Cancer | 0.123 | 13 | 0.0609 | 0.0169 | 0.002 |
| cancer | tissue | | | | | |
| | Adjacent | 0.052 | 13 | 0.0214 | 0.0059 | |
| | Tissue | | | | | |
| Total | Cancer | 0.248 | 58 | 0.3888 | 0.0510 | 0.000 |
| | Tissue | | | | | |
| | Adjacent | 0.057 | 58 | 0.0283 | 0.0037 | |
| | tissue | | | | | |

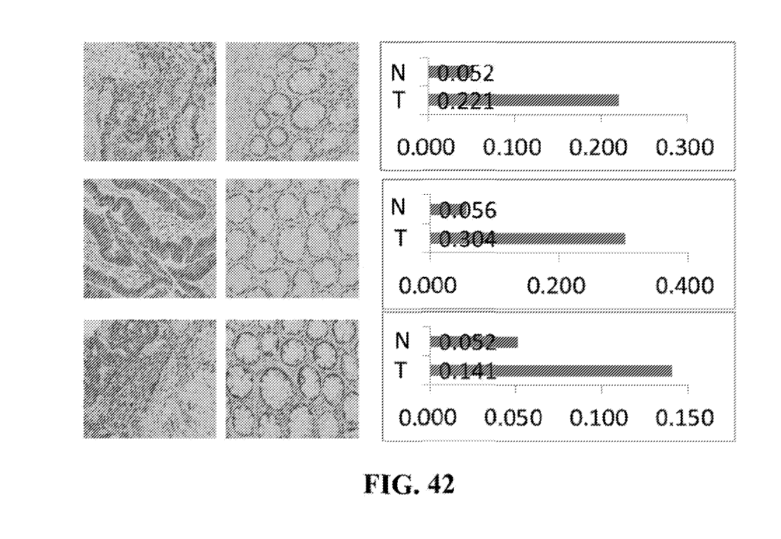

The DR5 expression of the cancer tissues and the relative adjacent tissues were determined by IHC, and compared with the CLEIA results. As shown in FIG. 42, there is a good relativity between CLEIA DR5 level and IHC results, and the DR5 expression detected by CLEIA Kit and IHC are compatible.

### Example 8. CTB006-Related Sequences

CTB006-related sequences are provided below.

The murine CTB006 light chain variable region nucleic acid sequence is shown in Table 27 below.

TABLE 27
Murine CTB006 light chain variable
region nucleic acid sequence

SEQ ID NO.: 1

| | |
|---|---|
| 1 | GACATCGTCATGACCCAATCTCACAAATTCATGTCCACTTCAGTAGGAGACAGGGTCAGC |
| 61 | ATCACCTGCAAGGCCAGTCAGGATGTGAGTACTGCTGTAGCCTGGTATCAACAAAAACCA |
| 121 | GGGCAATCTCCTAGACTACTGATTTACTGGGCATCCACCCGGCACACTGGAGTCCCTGAT |
| 181 | CGCTTCACAGGCAGTGGATCTGGGACAGATTATACTCTCACCATCAGCAGTGTGCAGGCT |
| 241 | GAAGACCAGGCACTTTATTACTGTCAGCAACATTATCGCACTCCGTGG |

The murine CTB006 light chain variable region amino acid sequence is shown in Table 28 below.

TABLE 28
Murine CTB006 light chain variable
region amino acid sequence

| SEQ ID NO: 2 | |
|---|---|
| 1 | D I V M T Q S H K F M S T S V G D R V S |
| 21 | I T C K A S Q D V S T A V A W Y Q Q K P |
| 41 | G Q S P R L L I Y W A S T R H T G V P D |
| 61 | R F T G S G S G T D Y T L T I S S V Q A |
| 81 | E D Q A L Y Y C Q Q H Y R T P W |

The murine CTB006 light chain variable region nucleic acid and amino acid sequence are shown in Table 29 below.

TABLE 29
Murine CTB006 light chain variable region
nucleic acid and amino acid sequence

| | |
|---|---|
| 1 | GACATCGTCATGACCCAATCTCACAAATTCATGTCCACTTCAGTAGGAGACAGGGTCAGC |
| 1 | D I V M T Q S H K F M S T S V G D R V S |
| 61 | ATCACCTGCAAGGCCAGTCAGGATGTGAGTACTGCTGTAGCCTGGTATCAACAAAAACCA |
| 21 | I T C K A S Q D V S T A V A W Y Q Q K P |
| 121 | GGGCAATCTCCTAGACTACTGATTTACTGGGCATCCACCCGGCACACTGGAGTCCCTGAT |
| 41 | G Q S P R L L I Y W A S T R H T G V P D |
| 181 | CGCTTCACAGGCAGTGGATCTGGGACAGATTATACTCTCACCATCAGCAGTGTGCAGGCT |
| 61 | R F T G S G S G T D Y T L T I S S V Q A |
| 241 | GAAGACCAGGCACTTTATTACTGTCAGCAACATTATCGCACTCCGTGG SEQ ID NO.: 1 |
| 81 | E D Q A L Y Y C Q Q H Y R T P W SEQ ID NO.: 2 |

The murine CTB006 light CDR1 amino acid sequence are shown in Table 30 below.

TABLE 30
Murine CTB006 light CDR1 amino acid sequence

| | |
|---|---|
| K A S Q D V S T A V A | SEQ ID NO: 3 |

The murine CTB006 light CDR2 amino acid sequence are shown in Table 31 below.

TABLE 31
Murine CTB006 light CDR2 amino acid sequence

| | |
|---|---|
| W A S T R H T | SEQ ID NO: 4 |

The murine CTB006 light CDR3 amino acid sequence are shown in Table 32 below.

TABLE 32
Murine CTB006 light CDR3 amino acid sequence

| | |
|---|---|
| Q Q H Y R T P W | SEQ ID NO: 5 |

The murine CTB006 heavy chain variable region nucleic acid sequence is shown in Table 33 below.

TABLE 33
Murine CTB006 heavy chain variable region nucleic acid sequence

| | | |
|---|---|---|
| 1 | CAGGTCCAACTGCAGCAGCCTGGACCTGAGCTGGTGAAGCCTGGGGCTTCAGTGAGGATG | |
| 61 | TCCTGCAAGGCTTCTGGCTACACCTTCACAAGCTACTTTATACATTGGGTGAAGCAGAGG | |
| 121 | CCTGGACAGGGACTTGAGTGGATTGGATGGATTTATCCTGGAAATGTTAATACTAAGTAC | |
| 181 | AGTGAGAAGTTCAAGGGTAAGGCCACACTGACTGCAGACAAATCCTCCAGCACAGCCTAC | |
| 241 | ATGCAGTTCAGCAGCCTGACCTCTGAGGACTCTGCGGTCTATTTCTGTGCAAGAGGGGAG | |
| 301 | GCTGGGTACTTTGAC | SEQ ID NO: 6 |

The murine CTB006 heavy chain variable region amino acid sequence is shown in Table 34 below.

TABLE 34
Murine CTB006 heavy chain variable region
amino acid sequence

| | | |
|---|---|---|
| 1 | Q V Q L Q Q P G P E L V K P G A S V R M | |
| 21 | S C K A S G Y T F T S Y F I H W V K Q R | |
| 41 | P G Q G L E W I G W I Y P G N V N T K Y | |
| 61 | S E K F K G K A T L T A D K S S S T A Y | |
| 81 | M Q F S S L T S E D S A V Y F C A R G E | |
| 101 | A G Y F D | SEQ ID NO: 7 |

The murine CTB006 heavy chain variable region nucleic acid and amino acid sequence is shown in Table 35 below.

TABLE 35
Murine CTB006 heavy chain variable region nucleic acid and
amino acid sequence

| | | |
|---|---|---|
| 1 | CAGGTCCAACTGCAGCAGCCTGGACCTGAGCTGGTGAAGCCTGGGGCTTCAGTGAGGATG | |
| 1 | Q V Q L Q Q P G P E L V K P G A S V R M | |
| 61 | TCCTGCAAGGCTTCTGGCTACACCTTCACAAGCTACTTTATACATTGGGTGAAGCAGAGG | |
| 21 | S C K A S G Y T F T S Y F I H W V K Q R | |
| 121 | CCTGGACAGGGACTTGAGTGGATTGGATGGATTTATCCTGGAAATGTTAATACTAAGTAC | |
| 41 | P G Q G L E W I G W I Y P G N V N T K Y | |
| 181 | AGTGAGAAGTTCAAGGGTAAGGCCACACTGACTGCAGACAAATCCTCCAGCACAGCCTAC | |
| 61 | S E K F K G K A T L T A D K S S S T A Y | |
| 241 | ATGCAGTTCAGCAGCCTGACCTCTGAGGACTCTGCGGTCTATTTCTGTGCAAGAGGGGAG | |
| 81 | M Q F S S L T S E D S A V Y F C A R G E | |
| 301 | GCTGGGTACTTTGAC | SEQ ID NO: 6 |
| 101 | A G Y F D | SEQ ID NO: 7 |

The murine CTB006 heavy CDR1 amino acid sequence is shown in Table 36 below.

TABLE 36
Murine CTB006 heavy CDR1 amino acid sequence

| | |
|---|---|
| S Y F I H | SEQ ID NO: 8 |

The murine CTB006 heavy CDR2 amino acid sequence is shown in Table 37 below.

TABLE 37
Murine CTB006 heavy CDR2 amino acid sequence

| | |
|---|---|
| W I Y P G N V N T K Y S E K F K G | SEQ ID NO: 9 |

The murine CTB006 heavy CDR3 amino acid sequence is shown in Table 38 below.

TABLE 38
Murine CTB006 heavy CDR3 amino acid sequence

| | |
|---|---|
| G E A G Y F D | SEQ ID NO: 10 |

The human chimeric CTB006 light chain nucleic acid sequence is shown in Table 39 below.

TABLE 39
Human chimeric CTB006 light chain nucleic acid sequence

| | | |
|---|---|---|
| 1 | ATGAGGCTCCCTGCTCAGCTCCTGGGGCTGCTAATGCTCTGGGTCTCTGGATCCAGTGGT | |
| 61 | GACATCGTCATGACCCAATCTCACAAATTCATGTCCACTTCAGTAGGAGACAGGGTCAGC | |
| 121 | ATCACCTGCAAGGCCAGTCAGGATGTGAGTACTGCTGTAGCCTGGTATCAACAAAAACCA | |
| 181 | GGGCAATCTCCTAGACTACTGATTTACTGGGCATCCACCCGGCACACTGGAGTCCCTGAT | |
| 241 | CGCTTCACAGGCAGTGGATCTGGGACAGATTATACTCTCACCATCAGCAGTGTGCAGGCT | |
| 301 | GAAGACCAGGCACTTTATTACTGTCAGCAACATTATCGCACTCCGTGGACGTTCGGTGGA | |
| 361 | GGCACCAAGCTGGAAATCAAACGGGCTGTGGCTGCACCATCTGTCGATATCTTCCCGCCA | |
| 421 | TCTGATGAGCAGTTGAAATCTGGAACTGCCTCTGTTGTGTGCCTGCTGAATAACTTCTAC | |
| 481 | CCCAGAGAGGCCAAAGTACAGTGGAAGGTGGATAACGCCCTCCAATCGGGTAACTCCCAG | |
| 541 | GAGAGTGTCACAGAGCAGGACAGCAAGGACAGCACCTACAGCCTCAGCAGCACCCTGACG | |
| 601 | CTGAGCAAAGCAGACTACGAGAAACACAAAGTCTACGCCTGCGAAGTTACCCATCAGGGC | |
| 661 | CTGAGCTCGCCCGTCACAAAGAGCTTCAACAGGGGAGAGTGTTAG | SEQ ID NO: 11 |

The human chimeric CTB006 light chain amino acid sequence is shown in Table 40 below.

TABLE 40
Human chimeric CTB006 light chain amino
acid sequence

| | | |
|---|---|---|
| 1 | M R L P A Q L L G L L M L W V S G S S G | |
| 21 | D I V M T Q S H K F M S T S V G D R V S | |
| 41 | I T C K A S Q D V S T A V A W Y Q Q K P | |
| 61 | G Q S P R L L I Y W A S T R H T G V P D | |
| 81 | R F T G S G S G T D Y T L T I S S V Q A | |
| 101 | E D Q A L Y Y C Q Q H Y R T P W T F G G | |
| 121 | G T K L E I K R A V A A P S V D I F P P | |
| 141 | S D E Q L K S G T A S V V C L L N N F Y | |
| 161 | P R E A K V Q W K V D N A L Q S G N S Q | |
| 181 | E S V T E Q D S K D S T Y S L S S T L T | |
| 201 | L S K A D Y E K H K V Y A C E V T H Q G | |
| 221 | L S S P V T K S F N R G E C | SEQ ID NO: 12 |

The human chimeric CTB006 light chain nucleic acid and amino acid sequence are shown in Table 41 below.

TABLE 41
Human chimeric CTB006 light chain nucleic acid and amino acid sequence

| | | |
|---|---|---|
| 1 | ATGAGGCTCCCTGCTCAGCTCCTGGGGCTGCTAATGCTCTGGGTCTCTGGATCCAGTGGT | |
| 1 | M R L P A Q L L G L L M L W V S G S S G | |
| 61 | GACATCGTCATGACCCAATCTCACAAATTCATGTCCACTTCAGTAGGAGACAGGGTCAGC | |
| 21 | D I V M T Q S H K F M S T S V G D R V S | |
| 121 | ATCACCTGCAAGGCCAGTCAGGATGTGAGTACTGCTGTAGCCTGGTATCAACAAAAACCA | |
| 41 | I T C K A S Q D V S T A V A W Y Q Q K P | |
| 181 | GGGCAATCTCCTAGACTACTGATTTACTGGGCATCCACCCGGCACACTGGAGTCCCTGAT | |
| 61 | G Q S P R L L I Y W A S T R H T G V P D | |
| 241 | CGCTTCACAGGCAGTGGATCTGGGACAGATTATACTCTCACCATCAGCAGTGTGCAGGCT | |
| 81 | R F T G S G S G T D Y T L T I S S V Q A | |
| 301 | GAAGACCAGGCACTTTATTACTGTCAGCAACATTATCGCACTCCGTGGACGTTCGGTGGA | |
| 101 | E D Q A L Y Y C Q Q H Y R T P W T F G G | |
| 361 | GGCACCAAGCTGGAAATCAAACGGGCTGTGGCTGCACCATCTGTCGATATCTTCCCGCCA | |
| 121 | G T K L E I K R A V A A P S V D I F P P | |
| 421 | TCTGATGAGCAGTTGAAATCTGGAACTGCCTCTGTTGTGTGCCTGCTGAATAACTTCTAC | |
| 141 | S D E Q L K S G T A S V V C L L N N F Y | |
| 481 | CCCAGAGAGGCCAAAGTACAGTGGAAGGTGGATAACGCCCTCCAATCGGGTAACTCCCAG | |
| 161 | P R E A K V Q W K V D N A L Q S G N S Q | |
| 541 | GAGAGTGTCACAGAGCAGGACAGCAAGGACAGCACCTACAGCCTCAGCAGCACCCTGACG | |
| 181 | E S V T E Q D S K D S T Y S L S S T L T | |
| 601 | CTGAGCAAAGCAGACTACGAGAAACACAAAGTCTACGCCTGCGAAGTTACCCATCAGGGC | |
| 201 | L S K A D Y E K H K V Y A C E V T H Q G | |
| 661 | CTGAGCTCGCCCGTCACAAAGAGCTTCAACAGGGGAGAGTGTTAG | SEQ ID NO: 11 |
| 221 | L S S P V T K S F N R G E C * | SEQ ID NO: 12 |

The human chimeric CTB006 heavy chain nucleic acid sequence is shown in Table 42 below.

TABLE 42
Human chimeric CTB006 heavy chain nucleic acid sequence

| | | |
|---|---|---|
| 1 | ATGGAGTTGGGGCTGAGCTGGGTTTTCCTTGTTGTTATATTAGAAGGTGTCCAGTGTGAG | |
| 61 | GTTCAGCTGCAGCAGTCTGGACCTGAGCTGGTGAAGCCTGGGGCTTCAGTGAGGATGTCC | |
| 121 | TGCAAGGCTTCTGGCTACACCTTCACAAGCTACTTTATACATTGGGTGAAGCAGAGGCCT | |
| 181 | GGACAGGGACTTGAGTGGATTGGATGGATTTATCCTGGAAATGTTAATACTAAGTACAGT | |
| 241 | GAGAAGTTCAAGGGTAAGGCCACACTGACTGCAGACAAATCCTCCAGCACAGCCTACATG | |
| 301 | CAGTTCAGCAGCCTGACCTCTGAGGACTCTGCGGTCTATTTCTGTGCAAGAGGGGAGGCT | |
| 361 | GGGTACTTTGACTACTGGGGCCAAGGCACCACTCTCACAGTCTCCTCAGCTAGCACCAAG | |
| 421 | GGCCCATCGGTCTTCCCCCTGGCGCCCTGCTCCAGGAGCACCTCTGGGGGCACAGCGGCC | |
| 481 | CTGGGCTGCCTGGTCAAGGACTACTTCCCCGAACCGGTGACGGTGTCGTGGAACTCAGGC | |
| 541 | GCCCTGACCAGCGGCGTGCACACCTTCCCGGCTGTCCTACAGTCCTCAGGACTCTACTCC | |
| 601 | CTCAGCAGCGTGGTGACCGTGCCCTCCAGCAGCTTGGGCACCCAGACCTACATCTGCAAC | |
| 661 | GTGAATCACAAGCCCAGCAACACCAAGGTGGACAAGAGAGTTGAGCCCAAATCTTGTGAC | |
| 721 | AAAACTCACACATGCCCACCGTGCCCAGCACCTGAACTCCTGGGGGGACCGTCAGTCTTC | |
| 781 | CTCTTCCCCCCAAAACCCAAGGACACCCTCATGATCTCCCGGACCCCTGAGGTCACATGC | |
| 841 | GTGGTGGTGGACGTGAGCCACGAAGACCCTGAGGTCAAGTTCAACTGGTACGTGGACGGC | |
| 901 | GTGGAGGTGCATAATGCCAAGACAAAGCCGCGGGAGGAGCAGTACAACAGCACGTACCGT | |
| 961 | GTGGTCAGCGTCCTCACCGTCCTGCACCAGGACTGGCTGAACGGCAAGGAGTACAAGTGC | |
| 1021 | AAGGTCTCCAACAAAGGCCTCCCAGCCCCCATCGAGAAAACCATCTCCAAAGCCAAAGGG | |
| 1081 | CAGCCCCGAGAACCACAGGTGTACACCCTGCCCCCATCCCGGGAGGAGATGACCAAGAAC | |
| 1141 | CAGGTCAGCCTGACCTGCCTGGTCAAAGGCTTCTATCCCAGCGACATCGCCGTGGAGTGG | |
| 1201 | GAGAGCAATGGGCAGCCGGAGAACAACTACAAGACCACGCCTCCCGTGCTGGACTCCGAC | |
| 1261 | GGCTCCTTCTTCCTCTATAGCAAGCTCACCATGGACAAGAGCAGGTGGCAGCAGGGGAAC | |
| 1321 | GTCTTCTCATGCTCCGTGATGCATGAGGCTCTGCACAACCACTACACGCAGAAGAGCCTC | |
| 1381 | TCCCTGTCTCCGGGTAAATGA | SEQ ID NO: 13 |

The human chimeric CTB006 heavy chain amino acid sequence is shown in Table 43 below.

TABLE 43
Human chimeric CTB006 heavy chain amino acid sequence

| | | |
|---|---|---|
| 1 | M E L G L S W V F L V V I L E G V Q C E | |
| 21 | V Q L Q Q S G P E L V K P G A S V R M S | |
| 41 | C K A S G Y T F T S Y F I H W V K Q R P | |
| 61 | G Q G L E W I G W I Y P G N V N T K Y S | |
| 81 | E K F K G K A T L T A D K S S S T A Y M | |
| 101 | Q F S S L T S E D S A V Y F C A R G E A | |
| 121 | G Y F D Y W G Q G T T L T V S S A S T K | |
| 141 | G P S V F P L A P C S R S T S G G T A A | |
| 161 | L G C L V K D Y F P E P V T V S W N S G | |
| 181 | A L T S G V H T F P A V L Q S S G L Y S | |
| 201 | L S S V V T V P S S S L G T Q T Y I C N | |
| 221 | V N H K P S N T K V D K R V E P K S C D | |
| 241 | K T H T C P P C P A P E L L G G P S V F | |
| 261 | L F P P K P K D T L M I S R T P E V T C | |
| 281 | V V V D V S H E D P E V K F N W Y V D G | |
| 301 | V E V H N A K T K P R E E Q Y N S T Y R | |
| 321 | V V S V L T V L H Q D W L N G K E Y K C | |
| 341 | K V S N K G L P A P I E K T I S K A K G | |
| 361 | Q P R E P Q V Y T L P P S R E E M T K N | |
| 381 | Q V S L T C L V K G F Y P S D I A V E W | |
| 401 | E S N G Q P E N N Y K T T P P V L D S D | |
| 421 | G S F F L Y S K L T M D K S R W Q Q G N | |
| 441 | V F S C S V M H E A L H N H Y T Q K S L | |
| 461 | S L S P G K * | SEQ ID NO: 14 |

The human chimeric CTB006 heavy chain nucleic acid and amino acid sequence are shown in Table 44 below.

TABLE 44
Human chimeric CTB006 heavy chain nucleic acid and amino acid sequence

| | | |
|---|---|---|
| 1 | ATGGAGTTGGGGCTGAGCTGGGTTTTCCTTGTTGTTATATTAGAAGGTGTCCAGTGTGAG | |
| 1 | M E L G L S W V F L V V I L E G V Q C E | |
| 61 | GTTCAGCTGCAGCAGTCTGGACCTGAGCTGGTGAAGCCTGGGGCTTCAGTGAGGATGTCC | |
| 21 | V Q L Q Q S G P E L V K P G A S V R M S | |
| 121 | TGCAAGGCTTCTGGCTACACCTTCACAAGCTACTTTATACATTGGGTGAAGCAGAGGCCT | |
| 41 | C K A S G Y T F T S Y F I H W V K Q R P | |
| 181 | GGACAGGGACTTGAGTGGATTGGATGGATTTATCCTGGAAATGTTAATACTAAGTACAGT | |
| 61 | G Q G L E W I G W I Y P G N V N T K Y S | |
| 241 | GAGAAGTTCAAGGGTAAGGCCACACTGACTGCAGACAAATCCTCCAGCACAGCCTACATG | |
| 81 | E K F K G K A T L T A D K S S S T A Y M | |
| 301 | CAGTTCAGCAGCCTGACCTCTGAGGACTCTGCGGTCTATTTCTGTGCAAGAGGGGAGGCT | |
| 101 | Q F S S L T S E D S A V Y F C A R G E A | |
| 361 | GGGTACTTTGACTACTGGGGCCAAGGCACCACTCTCACAGTCTCCTCAGCTAGCACCAAG | |
| 121 | G Y F D Y W G Q G T T L T V S S A S T K | |
| 421 | GGCCCATCGGTCTTCCCCCTGGCGCCCTGCTCCAGGAGCACCTCTGGGGGCACAGCGGCC | |
| 141 | G P S V F P L A P C S R S T S G G T A A | |
| 481 | CTGGGCTGCCTGGTCAAGGACTACTTCCCCGAACCGGTGACGGTGTCGTGGAACTCAGGC | |
| 161 | L G C L V K D Y F P E P V T V S W N S G | |
| 541 | GCCCTGACCAGCGGCGTGCACACCTTCCCGGCTGTCCTACAGTCCTCAGGACTCTACTCC | |
| 181 | A L T S G V H T F P A V L Q S S G L Y S | |
| 601 | CTCAGCAGCGTGGTGACCGTGCCCTCCAGCAGCTTGGGCACCCAGACCTACATCTGCAAC | |
| 201 | L S S V V T V P S S S L G T Q T Y I C N | |
| 661 | GTGAATCACAAGCCCAGCAACACCAAGGTGGACAAGAGAGTTGAGCCCAAATCTTGTGAC | |
| 221 | V N H K P S N T K V D K R V E P K S C D | |
| 721 | AAAACTCACACATGCCCACCGTGCCCAGCACCTGAACTCCTGGGGGGACCGTCAGTCTTC | |
| 241 | K T H T C P P C P A P E L L G G P S V F | |
| 781 | CTCTTCCCCCCAAAACCCAAGGACACCCTCATGATCTCCCGGACCCCTGAGGTCACATGC | |
| 261 | L F P P K P K D T L M I S R T P E V T C | |
| 841 | GTGGTGGTGGACGTGAGCCACGAAGACCCTGAGGTCAAGTTCAACTGGTACGTGGACGGC | |
| 281 | V V V D V S H E D P E V K F N W Y V D G | |
| 901 | GTGGAGGTGCATAATGCCAAGACAAAGCCGCGGGAGGAGCAGTACAACAGCACGTACCGT | |
| 301 | V E V H N A K T K P R E E Q Y N S T Y R | |
| 961 | GTGGTCAGCGTCCTCACCGTCCTGCACCAGGACTGGCTGAACGGCAAGGAGTACAAGTGC | |
| 321 | V V S V L T V L H Q D W L N G K E Y K C | |
| 1021 | AAGGTCTCCAACAAAGGCCTCCCAGCCCCCATCGAGAAAACCATCTCCAAAGCCAAAGGG | |
| 341 | K V S N K G L P A P I E K T I S K A K G | |
| 1081 | CAGCCCCGAGAACCACAGGTGTACACCCTGCCCCCATCCCGGGAGGAGATGACCAAGAAC | |
| 361 | Q P R E P Q V Y T L P P S R E E M T K N | |
| 1141 | CAGGTCAGCCTGACCTGCCTGGTCAAAGGCTTCTATCCCAGCGACATCGCCGTGGAGTGG | |
| 381 | Q V S L T C L V K G F Y P S D I A V E W | |
| 1201 | GAGAGCAATGGGCAGCCGGAGAACAACTACAAGACCACGCCTCCCGTGCTGGACTCCGAC | |
| 401 | E S N G Q P E N N Y K T T P P V L D S D | |
| 1261 | GGCTCCTTCTTCCTCTATAGCAAGCTCACCATGGACAAGAGCAGGTGGCAGCAGGGGAAC | |
| 421 | G S F F L Y S K L T M D K S R W Q Q G N | |
| 1321 | GTCTTCTCATGCTCCGTGATGCATGAGGCTCTGCACAACCACTACACGCAGAAGAGCCTC | |
| 441 | V F S C S V M H E A L H N H Y T Q K S L | |
| 1381 | TCCCTGTCTCCGGGTAAATGA | SEQ ID NO: 13 |
| 461 | S L S P G K * | SEQ ID NO: 14 |

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

1. A method for treating cancer in a subject in need thereof, comprising (a) administering to the subject a composition comprising a monoclonal antibody having the same epitope specificity as the mouse-mouse hybridoma CTB006 having CGMCC Accession No. 1691; and (b) simultaneously, sequentially or separately administering to the subject a chemotherapeutic agent selected from the group consisting of 5-fluorouracil, taxol, carboplatin, cisplatin, sorafenib, lapatinib, erlotinib, erbitux, herceptin, and irinotecan.

2. The method of claim 1, wherein the antibody comprises heavy chain CDR amino acid sequences SYFIH (SEQ ID NO: 8), WIYPGNVNTKYSEKFKG (SEQ ID NO: 9), and GEAGYFD (SEQ ID NO: 10), and light chain CDR amino acid sequences KASQDVSTAVA (SEQ ID NO: 3), WASTRHT (SEQ ID NO: 4), and QQHYRTPW (SEQ ID NO: 5).

3. (canceled)

4. The method of claim 1, wherein the antibody is a human chimeric antibody comprising the heavy chain amino acid sequence set forth in SEQ ID NO: 14 and the light chain amino acid sequence set forth in SEQ ID NO: 12.

5. The method of claim 1, wherein the cancer is selected from the group consisting of liver cancer, colon cancer, breast cancer, ovarian cancer, and leukemia.

6-8. (canceled)

9. A method for selectively inducing apoptosis in cells expressing a TRAIL-R2 polypeptide, comprising:
(a) identifying cells expressing the TRAIL-R2 polypeptide;
(b) contacting the cells with a monoclonal antibody having the same epitope specificity as the mouse-mouse hybridoma CTB006 having CGMCC Accession No. 1691; and
(c) contacting the cells with at least a chemotherapeutic agent selected from the group consisting of 5-fluorouracil, taxol, gemcitabine, carboplatin, cisplatin, sorafenib, lapatinib, erlotinib, erbitux, herceptin, and irinotecan.

10. The method of claim 9, wherein the antibody comprises heavy chain CDR amino acid sequences SYFIH (SEQ ID NO: 8), WIYPGNVNTKYSEKFKG (SEQ ID NO: 9), and GEAGYFD (SEQ ID NO: 10), and light chain CDR amino acid sequences KASQDVSTAVA (SEQ ID NO: 3), WASTRHT (SEQ ID NO: 4), and QQHYRTPW (SEQ ID NO: 5).

11. (canceled)

12. The method of claim 9, wherein the antibody is a human chimeric antibody comprising the heavy chain amino acid sequence set forth in SEQ ID NO: 14 and the light chain amino acid sequence set forth in SEQ ID NO: 12.

13. The method of claim 9, wherein the TRAIL-R2-expressing cells are cancer cells.

14. The method of claim 13, wherein the cancer cells are selected from the group consisting of liver cancer cells, colon cancer cells, breast cancer cells, ovarian cancer cells, and leukemia cells.

15-25. (canceled)

26. An in vitro method of identifying a subject amenable to cancer treatment comprising the method of claim 1, comprising contacting a tumor sample from the patient with a monoclonal antibody having the same epitope specificity as the mouse-mouse hybridoma CTB006 having CGMCC Accession No. 1691.

27. The method of claim 26, wherein the antibody comprises heavy chain CDR amino acid sequences SYFIH (SEQ ID NO: 8), WIYPGNVNTKYSEKFKG (SEQ ID NO: 9), and GEAGYFD (SEQ ID NO: 10), and light chain CDR amino acid sequences KASQDVSTAVA (SEQ ID NO: 3), WASTRHT (SEQ ID NO: 4), and QQHYRTPW (SEQ ID NO: 5).

28. (canceled)

29. The method of claim 26, wherein the antibody is a human chimeric antibody comprising the heavy chain amino acid sequence set forth in SEQ ID NO: 14 and the light chain amino acid sequence set forth in SEQ ID NO: 11 SEQ ID NO: 12.

30. The method of claim 26, wherein the cancer is selected from the group consisting of liver cancer, colon cancer, breast cancer, ovarian cancer, and leukemia.

31-52. (canceled)


SEQUENCE LISTING (15 sequences)

SEQ ID NO: 1 - DNA, 288 bp, Artificial Sequence
  Murine CTB006 light chain variable region
gacatcgtcatgacccaatctcacaaattcatgtccacttcagtaggagacagggtcagcatcacctgcaaggccagtcaggatgtgagtactgctgtagcctggtatcaacaaaaaccagggcaatctcctagactactgatttactgggcatccacccggcacactggagtccctgatcgcttcacaggcagtggatctgggacagattatactctcaccatcagcagtgtgcaggctgaagaccaggcactttattactgtcagcaacattatcgcactccgtgg

SEQ ID NO: 2 - protein, 96 aa, Artificial Sequence
  Murine CTB006 light chain variable region
DIVMTQSHKFMSTSVGDRVSITCKASQDVSTAVAWYQQKPGQSPRLLIYWASTRHTGVPDRFTGSGSGTDYTLTISSVQAEDQALYYCQQHYRTPW

SEQ ID NO: 3 - protein, 11 aa, Artificial Sequence
  Murine CTB006 light CDR1
KASQDVSTAVA

SEQ ID NO: 4 - protein, 7 aa, Artificial Sequence
  Murine CTB006 light CDR2
WASTRHT

SEQ ID NO: 5 - protein, 8 aa, Artificial Sequence
  Murine CTB006 light CDR3
QQHYRTPW

SEQ ID NO: 6 - DNA, 315 bp, Artificial Sequence
  Murine CTB006 heavy chain variable region
caggtccaactgcagcagcctggacctgagctggtgaagcctggggcttcagtgaggatgtcctgcaaggcttctggctacaccttcacaagctactttatacattgggtgaagcagaggcctggacagggacttgagtggattggatggatttatcctggaaatgttaatactaagtacagtgagaagttcaagggtaaggccacactgactgcagacaaatcctccagcacagcctacatgcagttcagcagcctgacctctgaggactctgcggtctatttctgtgcaagaggggaggctgggtactttgac

SEQ ID NO: 7 - protein, 105 aa, Artificial Sequence
  Murine CTB006 heavy chain variable region
QVQLQQPGPELVKPGASVRMSCKASGYTFTSYFIHWVKQRPGQGLEWIGWIYPGNVNTKYSEKFKGKATLTADKSSSTAYMQFSSLTSEDSAVYFCARGEAGYFD

SEQ ID NO: 8 - protein, 5 aa, Artificial Sequence
  Murine CTB006 heavy CDR1
SYFIH

SEQ ID NO: 9 - protein, 17 aa, Artificial Sequence
  Murine CTB006 heavy CDR2
WIYPGNVNTKYSEKFKG

SEQ ID NO: 10 - protein, 7 aa, Artificial Sequence
  Murine CTB006 heavy CDR3
GEAGYFD

SEQ ID NO: 11 - DNA, 705 bp, Artificial Sequence
  Human chimeric CTB006 light chain
atgaggctccctgctcagctcctggggctgctaatgctctgggtctctggatccagtggtgacatcgtcatgacccaatctcacaaattcatgtccacttcagtaggagacagggtcagcatcacctgcaaggccagtcaggatgtgagtactgctgtagcctggtatcaacaaaaaccagggcaatctcctagactactgatttactgggcatccacccggcacactggagtccctgatcgcttcacaggcagtggatctgggacagattatactctcaccatcagcagtgtgcaggctgaagaccaggcactttattactgtcagcaacattatcgcactccgtggacgttcggtggaggcaccaagctggaaatcaaacgggctgtggctgcaccatctgtcgatatcttcccgccatctgatgagcagttgaaatctggaactgcctctgttgtgtgcctgctgaataacttctaccccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagttacccatcagggcctgagctcgcccgtcacaaagagcttcaacaggggagagtgttag

SEQ ID NO: 12 - protein, 234 aa, Artificial Sequence
  Human chimeric CTB006 light chain
MRLPAQLLGLLMLWVSGSSGDIVMTQSHKFMSTSVGDRVSITCKASQDVSTAVAWYQQKPGQSPRLLIYWASTRHTGVPDRFTGSGSGTDYTLTISSVQAEDQALYYCQQHYRTPWTFGGGTKLEIKRAVAAPSVDIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - DNA, 1401 bp, Artificial Sequence
  Human chimeric CTB006 heavy chain
atggagttggggctgagctgggttttccttgttgttatattagaaggtgtccagtgtgaggttcagctgcagcagtctggacctgagctggtgaagcctggggcttcagtgaggatgtcctgcaaggcttctggctacaccttcacaagctactttatacattgggtgaagcagaggcctggacagggacttgagtggattggatggatttatcctggaaatgttaatactaagtacagtgagaagttcaagggtaaggccacactgactgcagacaaatcctccagcacagcctacatgcagttcagcagcctgacctctgaggactctgcggtctatttctgtgcaagaggggaggctgggtactttgactactggggccaaggcaccactctcacagtctcctcagctagcaccaagggcccatcggtcttccccctggcgccctgctccaggagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaacggcaaggagtacaagtgcaaggtctccaacaaaggcctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccatggacaagagcaggtggcagcaggggaacgtcttctcatgctccgtgatgcatgaggctctgcacaaccactacacgcagaagagcctctccctgtctccgggtaaatga

SEQ ID NO: 14 - protein, 466 aa, Artificial Sequence
  Human chimeric CTB006 heavy chain
MELGLSWVFLVVILEGVQCEVQLQQSGPELVKPGASVRMSCKASGYTFTSYFIHWVKQRPGQGLEWIGWIYPGNVNTKYSEKFKGKATLTADKSSSTAYMQFSSLTSEDSAVYFCARGEAGYFDYWGQGTTLTVSSASTKGPSVFPLAPCSRSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPAPIEKTISKAKGQPREPQVYTLPPSREEMTKNQVSLTCLVKGFYPSDIAVEWESNGQPENNYKTTPPVLDSDGSFFLYSKLTMDKSRWQQGNVFSCSVMHEALHNHYTQKSLSLSPGK

SEQ ID NO: 15 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS